SEQUENCE LISTING (37 sequences)

SEQ ID NO: 1 - DNA, 266 bp, Hepatitis C virus
  misc_feature (157)..(157): n = G or T
cactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtccnttcttggataaacccgctcacatgcctggagatttgggcgtgcccccgcaagactgctagccgagtagtgttgggtcgcgaaaggccttgtggtactgcctgatagggt

SEQ ID NO: 2 - DNA, 265 bp, Epstein Barr virus
cactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggt

SEQ ID NO: 3 - DNA, 226 bp, Hepatitis C virus
ccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgca

SEQ ID NO: 4 - DNA, 226 bp, Epstein Barr virus
ccaggaccccccctcccgggagagccatagtggtctgcggaaccggtgagtacaccggaattgccaggacgaccgggtcctttcttggataaatccgctcaatgcctggagatttgggcgtgcccccgcaagactgctagccgagtagtgttgggtcgcgaaaggccttgtggtactgcctgatagggtgcttgcgagtgctccgggaggtctcgtagaccgtgca

SEQ ID NO: 5 - DNA, 131 bp, Hepatitis C virus
cactcccctgtgaggaactactgtcttcacgcagaaagcgtctagccatggcgttagtatgagtgtcgtgcagcctccaggaccccccctcccgggagagccatagtggtctgcggaaccggtgagtacac

SEQ ID NO: 6 - DNA, 131 bp, Epstein Barr virus; identical to SEQ ID NO: 5

SEQ ID NO: 7 - DNA, 131 bp, Epstein Barr virus; identical to SEQ ID NO: 5

SEQ ID NO: 8 - DNA, 131 bp, Epstein Barr virus
cactcccctgtgaggaactactgtcttcacgcagaaagcgtctagccatggcgttagtatgagtgtcgtacagcctccaggcccccccctcccgggagagccatagtggtctgcggaaccggtgagtacac

SEQ ID NO: 9 - DNA, 131 bp, Epstein Barr virus; identical to SEQ ID NO: 5

SEQ ID NO: 10 - DNA, 131 bp, Epstein Barr virus; identical to SEQ ID NO: 5

SEQ ID NO: 11 - DNA, 171 bp, Hepatitis C virus
cggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgca

SEQ ID NO: 12 - DNA, 171 bp, Epstein Barr virus; identical to SEQ ID NO: 11

SEQ ID NO: 13 - DNA, 171 bp, Epstein Barr virus
cggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgca

SEQ ID NO: 14 - DNA, 171 bp, Epstein Barr virus
cggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgca

SEQ ID NO: 15 - DNA, 171 bp, Epstein Barr virus; identical to SEQ ID NO: 11

SEQ ID NO: 16 - DNA, 171 bp, Epstein Barr virus
cggaattgccaggacgaccgggtcctttcttggattaatccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgca

SEQ ID NO: 17 - DNA, 383 bp, Epstein Barr virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 18 - DNA, 26 bp, Hepatitis C virus
atgggggcgacactccaccatgaatc

SEQ ID NO: 19 - DNA, 25 bp, Hepatitis C virus
gttacgtttggtttttctttgaggt

SEQ ID NO: 20 - DNA, 24 bp, Hepatitis C virus
tgtcttcacgcagaaagcgtctag

SEQ ID NO: 21 - DNA, 23 bp, Hepatitis C virus
caagcaccctatcaggcagtacc

SEQ ID NO: 22 - DNA, 20 bp, Artificial Sequence
  Synthetic Oligonucleotide
gtggggcgccccaggcacca

SEQ ID NO: 23 - DNA, 24 bp, Artificial Sequence
  Synthetic Oligonucleotide
gtccttaatgtcacgcacgatttc

SEQ ID NO: 24 - DNA, 21 bp, Hepatitis C virus
gaagatgacttagatgttgtt

SEQ ID NO: 25 - DNA, 23 bp, Hepatitis C virus
aaccttgataaggtgccatgctt

SEQ ID NO: 26 - DNA, 21 bp, Artificial Sequence
  Synthetic Oligonucleotide
cgtctagccatggcgttaggt

SEQ ID NO: 27 - DNA, 17 bp, Artificial Sequence
  Synthetic Oligonucleotide
cctgcgcggcaagtaaa

SEQ ID NO: 28 - DNA, 18 bp, Artificial Sequence
  Synthetic Oligonucleotide
cgtcggagtcaacggatt

SEQ ID NO: 29 - DNA, 17 bp, Artificial Sequence
  Synthetic Oligonucleotide
gccatcacgccacagtt

SEQ ID NO: 30 - DNA, 20 bp, Artificial Sequence
  Synthetic Oligonucleotide
tcagcggaaccgggtgagta

SEQ ID NO: 31 - DNA, 21 bp, Artificial Sequence
  Synthetic Oligonucleotide
cggttggtgttacgtttggtt

SEQ ID NO: 32 - DNA, 17 bp, Artificial Sequence
  Synthetic Oligonucleotide
ttccatggcaccgtcaa

SEQ ID NO: 33 - DNA, 19 bp, Artificial Sequence
  Synthetic Oligonucleotide
gtccttccacgataccaaa

SEQ ID NO: 34 - DNA, 24 bp, Artificial Sequence
  Synthetic Oligonucleotide
tgggtcgcgaaaggccttgtggta

SEQ ID NO: 35 - DNA, 25 bp, Artificial Sequence
  Synthetic Oligonucleotide
tgcctgatagggtgcttgcgagtgc

SEQ ID NO: 36 - DNA, 25 bp, Artificial Sequence
  Synthetic Oligonucleotide
gctcctggaagatggtgatgggatt

SEQ ID NO: 37 - DNA, 26 bp, Artificial Sequence
  Synthetic Oligonucleotide
ccattgatgacaagcttcccgttctc

(19) United States
(12) Patent Application Publication
(10) Pub. No.: US 2007/0099179 A1
(43) Pub. Date: May 3, 2007

(54) Method for inducing hepatitis c virus (hcv) replication in vitro, cells and cell lines enabling robust hcv replication and kit therefor

(76) Inventors: Nahum Sonenberg, Cote-St-Luc (CA); Marcelo Lopez-Lastra, Santiago (CL)

Correspondence Address: GOUDREAU GAGE DUBUC, 2000 MCGILL COLLEGE, SUITE 2200, MONTREAL, QC, H3A 3H3, CA

(21) Appl. No.: 10/564,886

(22) PCT Filed: Jul. 14, 2004
(86) PCT No.: PCT/CA04/01009

§ 371 Date: Sep. 15, 2006

(30) Foreign Application Priority Data
Jul. 14, 2003 (CA) 2 436 104
Feb. 6, 2004 (CA) 2 454 540

(51) Int. Cl. C40B 30/06; C40B 40/02; C12Q 1/70; C12N 7/00

(52) U.S. Cl. 435/5; 435/235.1; 435/325

(57) ABSTRACT

The present invention relates to hepatitis C virus (HCV). More particularly, the invention relates to the development of a tool suitable for the search, discovery and validation of novel HCV antiviral drugs and therapies (e.g. vaccine). The invention further relates to methods for inducing HCV replication in vitro, and more particularly to a simple in vitro replication assay for HCV. In addition, the invention relates to the use of the methods of the present invention to prognose the resistance/sensitivity of a particular strain of HCV to a chosen anti-HCV agent. In one embodiment, the present invention relates to an adaptation of a therapeutic regimen for a patient infected with HCV which takes into account the resistance/sensitivity phenotype of the HCV strain which infects same. The invention more particularly, relates to a method for generating an established cell line which produces hepatitis C virus (HCV) comprising transforming peripheral blood mononuclear cells (PBMCs) which produce HCV with Epstein Barr virus (EBV). The invention also relates to an EBV established B-cell line capable of replicating complete and infectious HCV. As well, the invention relates to a cell-based in vitro replication system for HCV comprising an EBV-transformed B-cell capable of replicating complete and infectious HCV, and a second cell population having HCV tropism and in which robust HCV replication occurs, so that under appropriate culture conditions the second cell population can become infected by the infectious HCV produced by the EBV-transformed B-cell. The present invention also relates to kits for transforming a HCV-producing cell and to kits for diagnosing HCV in a patient.

### FIELD OF THE INVENTION

The present invention relates to hepatitis C virus (HCV). More particularly, the invention relates to the development of a tool suitable for the search, discovery and validation of novel HCV antiviral drugs and therapies (e.g. vaccine). The invention further relates to methods for inducing HCV replication in vitro, and more particularly to a simple in vitro replication assay for HCV. In addition, the invention relates to the use of the methods of the present invention to prognose the resistance/sensitivity of a particular strain of HCV to a chosen anti-HCV agent. In one embodiment, the present invention relates to an adaptation of a therapeutic regimen for a patient infected with HCV which takes into account the resistance/sensitivity phenotype of the HCV strain which infects same.

### BACKGROUND OF THE INVENTION

Hepatitis C virus (HCV) is a significant etiologic agent of chronic liver disease (1). It is estimated that more than 170 million people world-wide are seropositive (Hepatology 1997, 26:62S-65S). About 85% of primary infections become chronic, and ˜20% of patients with chronic HCV develop serious complications, such as liver cirrhosis, end-stage liver disease, hepatocellular carcinoma, and death due to liver failure (2). At this time HCV infection is one of the primary causes of liver transplantation in the US and other countries. Acute infections are usually subclinical or associated with mild symptoms, but the virus persists in more than 80% of infected individuals despite evidence of active, antiviral immunological response (Hepatol 1998, 28:939-944). . The long-term outcome of HCV persistent infections are varied, and they can range from an apparently healthy carrier state to chronic active hepatitis, liver cirrhosis, and eventually hepatocellular carcinoma (N Engl J Med 1992, 327:1899-1905). The mechanism of such pervasive persistence is unknown. To date, there is no vaccine for HCV and the most effective therapy is a treatment with peginterferon in combination with the nucleoside analogue ribavirin (Clin Liver Dis 7, 149-61 (2003), Nat Rev Drug Discov 1, 867-81 (2002). Unfortunately, during IFN-α treatment selection of viral variants resistant to INF-α occurs frequently (Microbes & Infection 200, 2:1743-1756). In addition, while ribavirin can be used to treat patients, HCV resistance thereto is also common. The search for HCV drugs as well as for the development of an HCV vaccine is severely hampered by the lack of an efficient tissue culture, of a robust cellular system that would support virus replication, or of a simple animal system for the study of replication and HCV pathogenicity. The only animal models currently available for the study of this virus are the chimpanzee and a mouse which possesses a chimeric human liver (Antiviral Research 2001, 52:1-17; Nat Med 2001, 7:927-933). These facts cast HCV as a human pathogen of extreme medical significance.

HCV is an enveloped RNA virus of the Flaviviridae family, classified within the Hepacivirus genus. It contains a 5′ uncapped positive strand RNA genome of 9.4 kb, that possesses two overlapping open reading frames: one is translated into a single polyprotein of 3010 aminoacids, while the other yields a 17 kDa protein (5-7). The viral polyprotein is processed to generate at least 10 different structural and nonstructural proteins (5, 6) (See FIGS. 1 and 2). The genome of HCV is highly heterogeneous and the virus circulates as quasi species in a single infected individual (8). HCV is primarily hepatotropic, but it has also been implicated in lymphoproliferative diseases such as mixed cryoglobulinaemia, B-cell non-Hodgkin's lymphoma, and Sjögren's syndrome (9). The case for HCV replication in PBLs is suggested by the following observations: a) PBLs from HCV positive donors are capable of transmitting viral infection when inoculated into chimpanzees (10), and b) HCV minus-strand RNA can be detected in PBLs from HCV carriers upon injection into SCID mice (11). However, despite the growing evidence that supports HCV entry into PBLs, viral RNA synthesis is still a matter of debate and virus replication in PBLs has not been demonstrated (9, 12). Detection of HCV genomic sequences (plus-strand) and replicative intermediates (minus-strand) in PBLs from chronically infected donors (13-16) or infected chimpanzees has been reported (17, 18). However, the presence of viral proteins or virus particles has never been documented.

There thus remains a need to provide a simple assay for HCV replication which would enable the study of HCV replication and/or pathogenesis and enable the development of a treatment or prophylaxis for HCV infections. There also remains a need to provide a HCV replication system which enables the screening, discovery and validation of novel anti-HCV compounds which can act in a larger number of stages of the HCV life cycle such as entry, replication, translation, assembly, trafficking and release. There also remains a need to provide a system which enables the replication of HCV from a patient so as to enable simpler and more efficient genotyping thereof and/or phenotyping (e.g. to identify its resistance/sensitivity characteristics toward anti-viral compounds). Patent and patent applications in the name of Virologic no. US20030008282A1 published Jan. 9, 2003; U.S. Pat. No. 6,242,187 issued Jun. 5, 2001; and U.S. Pat. No. 5,837,464 issued Nov. 17, 1998; describe exemplary methods for determining anti-viral drug susceptibility and resistance.

While HCV infects a large number of individuals, no efficient treatment or vaccine has been developed, despite a significant effort by the pharmaceutical industry. Thus, most companies with existing programs in the anti-infective area are focused towards the discovery of agents that are active against this virus. Until now, the human immunodeficiency virus (HIV) has provided a useful strategy for HCV antiviral drug development (Drug Discov. Today 1999, 4:518-529). In fact, the understanding of the function of anti-HIV drugs has outlined the research platform of most of the companies screening for anti-HCV drugs. Both viruses share interesting features. They lead to chronic infection, are highly mutable, and they code for specific enzymes that are not expected to be present in a normal non-infected cell. Based on the results of HIV therapy, it is likely that a combination therapy involving at least two drugs directed against separate targets will be more effective at reducing HCV load (quantity of virus in the serum), and minimizing the emergence of resistant strains than monotherapy. As the selected targets against HIV have been the viral encoded protease and the viral reverse transcriptase, it is not surprising to find that HCV protease and RNA dependent RNA polymerase have often been mentioned as candidate antiviral targets. As judged by the lack of disclosures, the discovery of anti-HCV agents has not been successful despite the functional similarity of several HCV-enzymes with known targets from other antiviral programs. Admittedly, part of this failure is because of the lack of a tissue culture system, which in turn limits primary screens to isolate viral protein targets. Interestingly, despite the fact that the enzyme assays to test HCV protease are known, the discovery of a potential drug candidate has met with little success. Taken together, it might be concluded that putative chemotypes for inhibition of HCV-targets are poorly represented in most industrial compound collections (Drug Discov. Today, 1999, 4:518-529).

Should a series of novel anti-HCV drugs be developed, to advance these agents into the drug development pipeline, several issues will need to be addressed, notably, their mechanism of action. Unfortunately, tissue culture and in vivo control experiments using whole virus are required to better determine the mode of inhibition. As stated above, an efficient cell culture system for the replication of HCV has not yet been provided (Drug Discov Today 1999, 4:518-529; Antiviral Res. 2001, 52:1-17; J. Mol. Biol. 2001, 313:451-464; Virus Res. 2002, 82:35-32).

Attempts have been made to provide an in vitro culture system for HCV, based on the use of human cells of hepatocytic and lymphocytic origin, but low and variable levels of replication and virus-induced cytotoxicity posed important problems. Primary hepatocytes (derived from a human donor) can be infected with HCV isolated from serum of infected patients, and the virus can be detected in the supernatant for several weeks after infection. HCV replication has been demonstrated by detection of minus-strand RNA, an intermediate of virus replication, in primary hepatocytes derived from a HCV-negative donor after infection with sera from HCV-positive patients. However, the availability of primary hepatocytes is limited. In addition, their isolation is time-consuming and labor-intensive. Consequently, such tissue culture systems are generally considered unsuitable for intensive large-scale antiviral studies.

Another example of progress in this domain has been the construction of subgenomic selective replicons cloned from a full-length HCV consensus genome from an infected liver (Antiviral Res. 2001, 52:1-17; J. Mol. Biol. 2001, 313:451-64; Virus Res. 2002, 82:25-32). Following transfection in human hepatoma cells, these RNAs were found to replicate to high levels, allowing detailed molecular studies of HCV and testing of antiviral drugs. One drawback of this system, however, is that it only expresses the non-structural viral proteins (Science 1999, 285:110-3). Therefore, studies aimed at assessing target viral assembly and trafficking through the cytoplasm cannot be carried out, with this reconstituted viral system. In other words, such artificial system is of a more limited potential to identify antiviral agents.

As previously mentioned, animal models currently exist to study HCV replication. Although the chimpanzee model has contributed significantly to the understanding of HCV infection, the high cost and availability of these animals limit the extent to which antiviral-drug or therapy studies can be carried out. Small laboratory animals, including mice, are not susceptible to infection with HCV. An alternative model such as a mouse model with a chimeric human liver has been generated (Nat Med 2001, 7:927-933). This system is considered laborious and is known to require special expertise to isolate and transplant human hepatocytes and maintain a colony of fragile immunodeficient mice with an approximately 35% mortality in newborns due to a defect in blood coagulation (Nature Med. 2001, 7:927-933). Nevertheless, when all the required conditions are met this mouse model can provide an interesting system for testing antiviral agents.

There thus remains a need to provide a simple in vitro system, which is suitable for the replication of HCV.

There also remains a need to provide an in vitro tissue culture system for the complete replication of HCV.

There further remains a need to provide a tissue culture system for HCV which enables the screening, discovery, validation and further development of drugs and therapies for essentially all the different stages of virus replication such as virus entry, replication [viral (−) and (+) strand synthesis], viral protein synthesis, virus assembly, virus trafficking, and virus release.

Also, there remains a need to provide a suitable cellular system which enables a quick enough assessment of the phenotype and/or genotype of one or more HCV infecting a patient, to adapt or improve the treatment thereof.

The present invention seeks to meet these and other needs.

The present description refers to a number of documents, the content of which is herein incorporated by reference in their entirety.

### SUMMARY OF THE INVENTION

The invention relates to a simple in vitro culture system, which is suitable for the full replication cycle of hepatitis C virus (HCV).

The invention further relates to immortalized cell lines and tissue culture systems using same, which enable the replication of complete HCV.

The invention also relates to a method for generating a cell line which is capable of generating enough HCV to enable characterization thereof (e.g. genotyping, phenotyping; structure-function relationship, . . . ). In a particular embodiment, the method comprises an immortalization of the cell line producing HCV.

The established cell lines of the present invention and others, established by a method of the present invention, can be used to assess the HCV replication and/or growth inhibiting effect of proteins (e.g. antibodies, other ligands, peptides) or nucleic acids (e.g. RNAi) expressed in these immortalized cell lines. Of course testing of such inhibitors is not limited to inhibitors which are expressed within these cells. Methods of expressing chosen nucleic acid sequences or peptide coding sequences in cells are well known in the art.

In addition, the invention relates to a tissue culture system for HCV which enables the screening, discovery, validation and further development of drugs and therapies for essentially all the different stages of virus replication such as virus entry, replication [viral (−) and (+) strand synthesis], viral protein synthesis, virus assembly, virus trafficking, and virus release. In a particular embodiment, the present invention enables a customization of screening, discovery, validation and/or development of drugs and/or therapies, based on a particular strain harbored by a patient. In one embodiment, the immortalization of peripheral mononuclear cells purified from a chronically infected HCV donor, and more particularly of the immortalization of a B-cell which enables robust replication of HCV within, enables simple and efficient production of HCV, characterization thereof, and opens. the way, amongst other things, to customized (and more general) screening methods, drug development, therapy development, diagnosis and prognosis development methods and assays.

The present invention also provides the means to diagnose HCV. In addition, it enables an identification of the response of a particular strain of HCV, from a particular patient, to a candidate antiviral compound or to a known antiviral compound.

The present invention further relates to a method of activating the replication of HCV in peripheral blood mononuclear cells (PBMCs) comprising obtention of same from a HCV-infection patient and activating the replication of HCV by incubating the PBMCs with an activation-inducing amount of at least one mitogen (e.g. activator).

The invention in addition relates to a co-culturing system for replicating HCV in vitro which comprises co-culturing PBMCs (or peripheral blood lymphocytes (PBLs)) infected with HCV, wherein the PBMCs have been activated and in which the HCV can actively replicate, together with a cell line, wherein the co-culturing enables infection of a naïve cell line and replication of the HCV thereinto. In a particular embodiment of the present invention, the cell line is an immortalized cell line.

The invention further relates to an in vitro co-culture system, which is suitable for the replication of hepatitis C virus (HCV), comprising: HCV-infected cells cultivated in the presence of an HCV-activating composition, said activating composition comprising at least one mitogen; and a non-infected cell type which is infectable with HCV, whereby said activating composition enables a full replication cycle of said HCV in both the originally infected cells and non-infected. cell type. In specific embodiments, the activating composition also comprises a cytokine. In more specific embodiments, the activating composition is selected from the group consisting of a) phytohaemagglutinin and IL-2; b) Staphylococcus aureus crown I (SAC) and IL-4; and c) SAC, IL2 and IL-4. The activation or stimulation of the EBV-established cell lines of the invention can also be effected by the activators, activating compositions and stimulators listed above, in reference to the co-culturing system.

In a particular embodiment, the invention provides the means to assess for sensitivity or resistance of a particular HCV strain to a known antiviral compound or candidate antiviral compound. In a related embodiment, such assessment enables an adaptation of the therapeutic regimen to better suit the sensitivity profile of the particular HCV strain. In a particular embodiment, the sensitivity/resistance of HCV can be assessed in the co-culturing system and with the immortalized cell lines of the present invention.

In a specific embodiment, there is provided an assay for screening a test agent and selecting an agent which possesses anti-HCV activity, comprising: a) growing a HCV infected cell according to an in vitro assay of the present invention; and b) assaying replication, translation, assembly infection or the like of HCV. In another embodiment, the immortalized cell lines of the invention are used. In yet another embodiment, the results with the co-culture system and immortalized cell lines are compared.

In another particular embodiment; the invention provides a method for identifying, from a library of compounds, a compound with anti-HCV activity, comprising: a) providing a screening assay comprising a measurable biological activity of HCV; b) contacting said screening assay with a test compound; and c) detecting if said test compound inhibits the biological activity of HCV; wherein a test compound which inhibits said biological activity is a compound with said inhibitory effect. In a specific embodiment of such method, the test compound with the therapeutic effect is further modified by combinatorial or medicinal chemistry to provide further analogs of the test compound also having the therapeutic effect. Different biological activities can be assessed with the co-culturing system and/or the immortalized cell lines of the present invention.

In another particular embodiment, the invention provides a method for identifying compound having therapeutic effect on HCV, comprising: a) providing a screening assay comprising a measurable biological activity of HCV; b) contacting the screening assay with a test compound; and c) detecting if the test compound inhibits the biological activity of HCV, wherein a test compound which inhibits the biological activity is a compound with this inhibitory effect. In a specific embodiment, the compound with the therapeutic effect is further modified by combinatorial or medicinal chemistry to provide analogs of the compound also having the therapeutic effect.

In another embodiment, the invention enables the phenotyping and/or genotyping of a particular HCV strain.

The invention in addition relates to a method of generating a vaccine to HCV comprising a use of an immortalized cell line of the present invention or a method to immortalize a HCV-producing cell line. In a more specific embodiment, the method further comprises a selection of clonal T cell populations that are responsive to the virus and an injection of these HCV responsive T-cell populations to the original donor or to other individuals.

It is believed that the Applicant is the first to provide an in vitro cell system which enables replication of a native HCV. It is also believed that the present inventors are the first to provide a method to immortalize a cell which harbors HCV and enables full replication thereof, as well as immortalized cell lines which enable replication of HCV through full replication cycles.

It is believed that prior to the present invention, while HCV was known to infect PBMCs, it was unknown that it could actively replicate in them. Thus, the present invention demonstrates HCV tropism for PBMCs and more particularly for PBLCs. As known in the art, PBMCs are a mixture of cells which also include macrophages and PBLCs (which can be obtained from PBMCs and contain about 85% of T cells, and about 5% of B cells, as estimated from non-infected patients).

It is also believed that the present invention provides the first demonstration that the HCV produced in an in vitro system is infectious and that sustainable replication of HCV can be achieved.

Before the present invention, large-scale production of HCV was unthinkable. The methods and in vitro system of the present invention enables active replication of HCV in primary cells for 7 to 9 days depending on the host cells and opens the way to large scale production. In any event, the instant invention enables sufficient replication to enable characterization thereof at the genetic and functional level. It also provides the means to increase the sensitivity of detection, by enabling replication of HCV and hence by increasing the quantity of same to be detected. In one particular embodiment, it enables detection of HCV protein expression.

It is believed that, prior to the present invention, no tissue culture technology currently existed to replicate HCV. The only animal models currently available for the study of this virus are the chimpanzee and mice models (mice with chimeric human livers). These animal based-systems are laborious and require special expertise and facilities.

In accordance with the present invention in one embodiment, there is thus provided a method for generating an established cell line which produces hepatitis C virus (HCV) comprising transforming peripheral blood mononuclear cells (PBMCs) which produce HCV with Epstein Barr virus (EBV). In a particular embodiment the cells producing HCV, are in a peripheral blood lymphocyte (PBL) fraction of said PBMCs. In yet another embodiment, the established HCV producing cells are B-cells. One advantage of the present invention is that the HCV produced by the established cell line enables the replication of complete and infectious HCV.

In accordance with another embodiment there is provided a method for producing HCV in vitro comprising generating an established cell line which produces hepatitis C virus (HCV) by transforming a B-cell which produces HCV with Epstein Barr virus (EBV) and growing the EBV-immortalized B-cell thereby obtained, in culture, under conditions enabling HCV production. In one embodiment, the stimulation of the EBV-immortalized B-cell to produce HCV, is carried-out with one of the stimulators or activators described herein. In one particular embodiment, this stimulating composition comprises one or more mitogen. In one particular embodiment, the B-cell which is transformed is present in a PBMC or PBL fraction. In one embodiment, the PBMC or PBL fraction is obtained from a HCV positive patient which is immunosuppressed (due to injection drug use (IDU) of said patient, or otherwise). In another particular embodiment, the PBMC or PBL fraction is obtained from a HCV positive patient which has not been treated with interleukin. In accordance, with one embodiment of the present invention, the method of producing HCV in vitro further comprises a co-cultivation of the EBV-immortalized B-cell with a second cell population or cell line having HCV tropism. Numerous examples of such cells or cell populations are exemplified herein, notably Huh-7, MT-4, Daudi cells and monocyte-derived dendritic cells (DCs). The use of DCs provides the advantage of enabling a vaccine preparation technology. In one embodiment the EBV-immortalized B-cell is stimulated by using an HCV replication activating-inducing amount of at least one mitogen.

In accordance with yet another embodiment, there is provided an EBV-established B-cell line capable of replicating complete and infectious HCV.

In accordance with yet another embodiment of the present invention, there is provided a cell-based in vitro replication system for HCV comprising an EBV-transformed B-cell capable of replicating complete and infectious HCV, and a second cell population having HCV tropism and in which robust HCV replication occurs, so that under appropriate culture conditions the second cell population can become infected by the infectious HCV produced by the EBV-transformed B-cell. In one such embodiment, the cell-based replication system further comprises appropriate culture media reagents conditions which enable infection of the second cell population (e.g. PBMs, PBLs, cell line having a tropism for HCV).

In accordance with yet an additional embodiment of the invention, there is provided an assay for screening a test agent and selecting an agent which possesses anti-HCV activity, comprising growing an EBV-immortalized cell line which produces HCV, or culturing the EBV-immortalized cell line with a second cell population so as produce HCV from the second cell population; and assaying a biological function of the HCV produced from the cell line or the cell population. As taught herein, numerous biological assay methods are known to the skilled artisan. In addition, as taught herein numerous biological functions can be assayed, non-limiting examples thereof include binding to a cellular receptor of HCV, replication, translation, assembly, and infectivity.

### DEFINITIONS

In the description that follows, a number of terms used in DNA technology are extensively utilized. In order to provide a clear and consistent understanding of the specification and claims, including the scope to be given such terms, the following definitions are provided.

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), Rieger et al., Glossary of genetics: Classical and molecular, 5^{th} edition, Springer-Verlag, New-York, 1991; Alberts et al., Molecular Biology of the Cell, 4^{th} edition, Garland science, New-York, 2002; and, Lewin, Genes VII, Oxford University Press, New-York, 2000. Generally, the procedures of cell cultures, infection, molecular biology methods and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. (1994, Current Protocols in Molecular Biology, John Wiley & Sons, New-York).

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. Routinely a 10% to 15% deviation preferably 10% is within the scope of the term “about”.

The terminology “appropriate culture media”, suitable growth conditions”, “suitable growth reagents” or the like is meant to refer to conditions and reagents which are suitable for the growth of cells which are grown in accordance with the present invention. Numerous media and growth conditions are known to the person of ordinary skill who can adapt same according to the particular cell type, population of cells, whether HCV production is to be stimulated, or the like. Examples of growth reagents are taught herein, but the present invention should not be so limited.

Isolated Nucleic Acid Molecule. An “isolated nucleic acid molecule”, as is generally understood and used herein, refers to a polymer of nucleotides, and includes but should not be limited to DNA and RNA. The “isolated” nucleic acid molecule is purified from its natural in vivo state.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense, RNAi also known as siRNA.]). RNA interference (RNAi) can be used in accordance with the present invention using, for example, the teachings of U.S. Pat. No. 6,506,559.

The term “recombinant DNA” as known in the art refers to a DNA molecule resulting from the joining of DNA segments. This is often referred to as genetic engineering. The same is true for “recombinant nucleic acid”.

DNA Segment. A DNA segment, as is generally understood and used herein, refers to a molecule comprising a linear stretch of nucleotides wherein the nucleotides are present in a sequence that can encode, through the genetic code, a molecule comprising a linear sequence of amino acid residues that is referred to as a protein, a protein fragment or a polypeptide.

Gene. A DNA The term “gene” is well known in the art and relates to a nucleic acid sequence which usually defines a single protein or polypeptide. In this context, a “structural gene” usually defines a DNA sequence which is transcribed into RNA and translated into a protein having a specific amino acid sequence thereby giving rise to a specific polypeptide or protein. It will be readily recognized by the person of ordinary skill, that the nucleic acid sequences of the present invention can be incorporated into anyone of numerous established kit formats which are well known in the art. It should also be understood that in view of the occurrence of alternative splicing or other mRNA editing processes, or protein editing, more than one protein or polypeptide can be encoded from one gene. Thus, the term “gene”, as used herein, should not be limited to genes which only encode one protein. Furthermore, in the context of the present invention HCV being an RNA virus, the definition of gene, structural gene and the like also refer to an RNA nucleic acid sequence. When the gene encodes a polypeptide, the polypeptide can be encoded by a full-length sequence or any portion of the coding sequence, so long as one functional activity of the protein is retained.

Complementary DNA (cDNA). Recombinant nucleic acid molecules synthesized by reverse transcription of messenger RNA (“RNA”).

Structural Gene. A DNA sequence that is transcribed into RNA that is then translated into a sequence of amino acids characteristic of a specific polypeptide.

Agarose Gel Electrophoresis. The most commonly used technique (though not the only one) for fractionating double strand DNA is agarose gel electrophoresis. The principle of this method is that DNA molecules migrate through the gel as though it were a sieve that retards the movement of the largest molecules to the greatest extent and the movement of the smallest molecules to the least extent. Note that the smaller the DNA fragment, the greater the mobility under electrophoresis in the agarose gel.

The DNA fragments fractionated by agarose gel electrophoresis can be visualized directly by a staining procedure if the number of fragments included in the pattern is small. In order to visualize a small subset of these fragments, a methodology referred to as the Southern hybridization procedure can be applied.

Southern Transfer Procedure. The purpose of the Southern transfer procedure (also referred to as blotting) is to physically transfer DNA fractionated by agarose gel electrophoresis onto a nitrocellulose filter paper or another appropriate surface or method, while retaining the relative positions of DNA fragments resulting from the fractionation procedure. The methodology used to accomplish the transfer from agarose gel to nitrocellulose involves drawing the DNA from the gel into the nitrocellulose paper by capillary action.

Nucleic Acid Hybridization. Nucleic acid hybridization depends on the principle that two single-stranded nucleic acid molecules that have complementary base sequences will reform the thermodynamically favored double-stranded structure if they are mixed under the proper conditions. The double-stranded structure will be formed between two complementary single-stranded nucleic acids even if one is immobilized on a nitrocellulose filter. In the Southern hybridization procedure, the latter situation occurs. As noted previously, the DNA of the individual to be tested is digested with a restriction endonuclease, fractionated by agarose gel electrophoresis, converted to the single-stranded form, and transferred to nitrocellulose paper, making it available for reannealing to the hybridization probe. Examples of hybridization conditions can be found in Ausubel, F.M. et al., Current protocols in Molecular Biology, John Wily & Sons, Inc., New York, N.Y. (1994). A nitrocellulose filter is incubated overnight at 42° C. with labeled probe in a solution containing 50% formamide, (or at 68° C. without formamide) high salt (either 5× SSC[20×: 3M NaCl/0.3M trisodium citrate] or 5× SSPE [20×: 3.6M NaCl/0.2M NaH2PO4/0.02M EDTA, pH 7.7]), 5× Denhardt's solution, 1% SDS, and 100 pμg/ml denatured salmon sperm DNA. This is followed by several washes in 0.2× SSC/0.1% SDS at a temperature selected based on the desired stringency: room temperature (low stringency), 42° C. (moderate stringency) or 68° C. (high stringency). The temperature selected is determined based on the melting temperature (™) of the DNA hybrid. Formamide can also be used in the washings and the temperature is adapted in accordance with the desired ™.

Hybridization Probe. To visualize a particular DNA sequence in the Southern hybridization procedure (e.g. an amplification product), a labeled DNA molecule or hybridization probe is reacted to the fractionated DNA bound to the nitrocellulose filter. The areas on the filter that carry DNA sequences complementary to the labeled DNA probe become labeled themselves as a consequence of the re-annealing reaction. The areas of the filter that exhibit such labeling are visualized. The hybridization probe is generally produced by molecular cloning of a specific DNA sequence. Knowing the sequences which are to be detected, the numerous primers and probes which can be designed and used in the context of the present invention can be readily determined by a person of ordinary skill in the art to which the present invention pertains.

Oligonucleotide, Oligomer or oligo. A molecule comprised of two or more deoxyribonucleotides or ribonucleotides, preferably more than three. Its exact size will depend on many factors, which in turn depend on the ultimate function or use of the oligonucleotide. An oligonucleotide can be derived synthetically or by cloning. Chimeras of deoxyribonucleotides and ribonucleotides may also be within the scope of the present invention.

Sequence Amplification. A method for generating large amounts of a target sequence. In general, one or more amplification primers are annealed to a nucleic acid sequence. Using appropriate enzymes, sequences found adjacent to, or in between the primers are amplified.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

Amplification Primer or Primer. An oligonucleotide which is capable of annealing adjacent to a target sequence and serving as an initiation point for DNA synthesis when placed under conditions in which synthesis of a primer extension product which is complementary to a nucleic acid strand is initiated. Primers can be, for example, designed to be specific for certain strains of HCV or chosen regions of HCV genome. In accordance with one embodiment of the present invention, the use of an “allele” or strain-specific primer with the other necessary reagents would give rise to an amplification product only when the “allele” or strain-specific sequence associated with a particular genotype or phenotype of HCV is present in the sample. In such an embodiment, the “wild type” allele would not give rise to an amplicon.

Antisense nucleic acid molecule. An “antisense nucleic acid molecule” refers herein to a molecule capable of forming a stable duplex or triplex with a portion of its targeted nucleic acid sequence (DNA or RNA). The design and modification of antisense nucleic acid molecules is well known in the art as described for example in WO 96/32966, WO 96/11266, WO 94/15646, WO 93/08845, and U.S. Pat. No. 5,593,974. Antisense nucleic acid molecules, as sense oligos, can be derived from the nucleic acid sequences of the present invention and modified in accordance to well known methods. For example, some antisense molecules (or sense oligos or sequences) can be designed to be more resistant to degradation, or if required, to increase their affinity to their targeted sequence, to affect their transport to chosen cell types or cell compartments, and/or to enhance their lipid solubility by using nucleotide analogs and/or substituting chosen chemical fragments thereof, as commonly known in the art. More recently, antisense nucleic acid molecules are often referred to as RNAi (see above or siRNA). In one embodiment, antisense nucleic acid molecules or RNAi are used to decrease or abrogate the expression of a HCV nucleic acid or proteins of a cellular nucleic acid or protein which is implicated in the HCV replication cycle.

While the present invention invention is examplified with established EBV-transformed B-cell lines, the invention should not be so limited. Indeed, any B-cell transforming virus, transforming gene or sequence thereof or any B-cell transforming means can be used in accordance with the present invention.

As used herein the terms “activator” and “inducer” refer to molecules which can trigger HCV replication in the culture system of the present invention. Inducement of HCV replication in the patient's infected cells require activation. This activation can be effected by a number of molecules. Non-limiting examples of mitogens which can be used as activators include receptor mediated activators and receptor independent activator such as: for T-cells: phytohaemagglutinin (PHA), concanavalin A, pokeweed, phorbolester, anti-CD3, superantigens, antigens that are presented by APC; for B-Cells: SAC, Staphilococcal protein A, CD40 ligand, antiimmunoglobulins, bacterial lipopolysaccharides (LPS). Cytokines such as for example IL2, IL4, IL5, IL6, IL10, IL13 can also be used to further induce HCV replication. In one embodiment, there is used a mixture of activators such as PHA and IL-2; SAC and IL-4, SAC and IL2 and IL-4. In order to activate the infected cell, at least one mitogen can be used. A cocktail of at least one mitogen with at least one cytokine was shown to trigger significant activation of HCV replication. IFN could also be used.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail above. As commonly known in the art, the oligos are designed to bind to a complementary sequence under selected conditions.

The nucleic acid (e.g. DNA or RNA) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 2000, Molecular Cloning—A Laboratory Manual, 3^{rd} Edition, CSH Laboratories; Ausubel et al., 1994, in Current Protocols in Molecular Biology, John Wiley & Sons Inc., N.Y.).

More specifically, the term “DNA” molecule or sequence (as well as sometimes the term “oligonucleotide”) refers to a molecule comprised generally of the deoxyribonucleotides adenine (A), guanine (G), thymine (T) and/or cytosine (C), often in a double-stranded form, and can comprise or include a “regulatory element” as known in the art or according to the present invention, as the term is defined herein. The term “oligonucleotide” or “DNA” can be found in linear DNA molecules or fragments, viruses, plasmids, vectors, chromosomes or synthetically derived DNA.

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²p, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma 32P ATP and polynucleotide kinase, using the Klenow fragment of Pol I of E. coli in the presence of radioactive dNTP (e.g. uniformly labeled DNA probe using random oligonucleotide primers in low-melt gels), using the SP6BT7 system to transcribe a DNA segment in the presence of one or more radioactive NTP, and the like.

A “heterologous” (e.g. a heterologous gene) region of a nucleic acid molecule is a subsegment of a nucleic acid molecule within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, 13-galactosidase, and the like which can be juxtaposed or joined to heterologous control regions or to heterologous polypeptides.

The term “vector” is commonly known in the art and defines a plasmid DNA, phage DNA, viral DNA, viral RNA, and the like, which can serve as a DNA or RNA vehicle into which DNA or RNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more. Of course, as known in the art, the term “expression” when relating to an RNA virus is similar but not identical to the expression of a DNA gene.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is preferably bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with SI nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

As used herein, the designation “functional derivative” denotes, in the context of a functional derivative of a sequence whether a nucleic acid or amino acid sequence, a molecule that retains a biological activity (either functional or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

It should be understood that some variants of protein or nucleic acid molecule of the invention might have substantially dissimilar biological interaction with a particular compound as compared to a “wild type” counterpart. For example, a particular mutation might render the HCV strain resistant to a particular compound or group of compounds. Also, a variant might have an improved or decreased function as compared to the wild-type or as compared to another HCV strain.

The functional derivatives of the present invention can be synthesized chemically or produced through recombinant DNA technology. As shown herein, they can also be isolated from clinical samples or isolated through culturing methods. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, and as alluded to above, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions associated with HCV infection. Alternatively, the molecules identified in accordance with the teachings of the present invention find utility in the development of more efficient anti-HCV compounds.

In one embodiment, the level of gene expression of a reporter gene (e.g. the level of luciferase, or β-gal, produced) fused to HCV sequences within cells treated with a candidate molecule(s) can be compared to that of the reporter gene in the absence of the molecules(s). The difference between the levels of gene expression indicates whether the molecule(s) of interest influences HCV replication. The magnitude of the level of reporter gene product expressed (treated vs. untreated cells) provides a relative indication of the strength of that molecule(s) as an anti-HVC compound.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). RNA transfection as known in the art is also possible.

In general, techniques for preparing antibodies (including monoclonal antibodies and hybridomas) and-for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents.

The present invention relates to a kit for diagnosing or prognosing HCV infection or response to HCV to a chosen therapeutic regimen comprising a use of a culturing system, or of a cell line of the present invention. For example, a compartmentalized kit in accordance with the present invention includes any kit in which reagents are contained in separate containers. In one embodiment, such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (e.g. HCV nucleic acid), a container which contains the primers used in the assay to genotype chosen regions of the HCV genome, containers which contain enzymes, containers which contain wash reagents, and containers which contain the reagents used to detect the extension products.

Yet in another embodiment, the present invention relates to an assay to screen for drugs for the treatment and/or prevention of HCV infection. In a particular embodiment, such assays can be designed using cells from patients infected with HCV having a known or unknown genotype.

In accordance with the present invention, there is also provided a method for identifying, from a library of compounds, a compound with therapeutic effect on HCV infection comprising providing a screening assay comprising a measurable biological activity of a HCV protein or gene (e.g. “in vitro”) or measuring infectivity, (viral release etc . . .), contacting the screening assay whether in vitro or “cellular” with a test compound; and detecting if the test compound modulates the biological activity of the protein or gene or the infectivity of the virus; wherein a test compound which modulates the biological activity or the infectivity is a compound with this therapeutic effect.

As used herein, “biological activity” refers to any detectable biological activity of a HCV gene or protein. This includes any physiological function attributable to a HCV gene or protein. Non-limiting examples include interaction of HCV macromolecules to another HCV macromolecule or to a host cell macromolecule, an enzymatic assay, expression of a sequence, nucleic and/or protein, infectivity . . .

In one embodiment, the invention provides assays for screening candidate or test compounds which interact with HCV genes or proteins.

In one embodiment, an assay is a cell-based assay in which a cell activity producing HCV is contacted with a test compound and the ability of the test compound to modulate the infectivity of HCV at different steps in the HCV complete life cycle, (e.g., attachment, entry into cells, replication, maturation etc).

The assays described above may be used as initial or primary screens to detect promising lead compounds for further development. Often, lead compounds will be further assessed in additional, different screens. Therefore, this invention also includes secondary anti-HCV screens which may involve purified HCV factors.

Tertiary screens may involve the study of the identified modulators in animal models for HCV infection. Accordingly, it is within the scope of this invention to further use an agent identified as described herein in an appropriate animal model. For example, an test compound identified as described herein can be used in an animal model to determine the efficacy, toxicity, or side effects of treatment with such an agent.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be routinely found in the art for references in such methods and libraries see WO 01/38564, for example.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the hepatitis C virus (HCV) genome organization (Prior art).

FIG. 2 shows the hypothetical model of the HCV replication cycle (Prior art).

FIG. 3 is a schematic representation of an experimental protocol. All experiments being performed with 1,000,000 cells/ml. T1=anti-CD3 (1 μg/μl final), IL-2 (final=200 U). T2=PHA (3 μg/μl), IL-2. T3=PHA, IL-2, SAC (1/104). T4=PHA, IL-2, SAC, IL-4 (final=200 U).

FIG. 4 is a schematic representation of PBMC and PBLC purification from blood samples.

FIG. 5 shows the detection of HCV NS3 and NS5 proteins by Western blot, in cell extracts from-treated PBMC from a HCV(+) patient.

FIG. 6 shows a validation that the antibody used is decorating the NS3 translated (if positive) in the replicon system and in accordance with one embodiment of the present invention activated (A) or non-activated (NA).

FIG. 7 shows the time course of HCV-NS3 detection: PBMCs from patient MLL-001.

FIG. 8 similarly to FIG. 7, shows the time course of HCV-NS3 detection: PBMCs from patient MLL-002.

FIG. 9 shows the detection of HCV-NS3 protein in treated (N3) PBMCs from HCV9+ donors.

FIG. 10 shows the detection of virus like particles by scanning electron microscopy.

FIG. 11 shows the electron microscopy of activated PBLs and detection of virus like particles.

FIG. 12 is a schematic representation of one embodiment of a virus partial purification protocol according to the present invention.

FIG. 13 shows the detection of HCV core protein in supernatant of treated (T) PBMC as compared to (NT), from an HCV(+) patient as compared to HCV(−) patient.

FIG. 14 shows RNA quantification I (virus copies/ng total RNA together with core detection in the treated (T) but not in the non-treated (NT) HCV(+) patients).

FIG. 15 is a schematic representation of one embodiment of the co-culture HCV infection assay system as compared to HCV(−) patient.

FIG. 16 shows infection of MT-4 cells RNA quantification II (virus copies/ng total RNA) together with core detection in the treated (T) but not in the non-treated (NT) HCV(+) patients); the correlation between HCV RNA detection in the target cell (in this case MT-4 cells) and treatment is also shown.

FIG. 17 shows co-culture of Huh-7 and HCV(−) PBMCs (control experiments).

FIG. 18 shows co-culture of Huh-7 and HCV(+) PBMCs (SB006) clearly demonstrating viral infection in panels (5-6_M as compared to 2 and 3 (NT).

FIG. 19 is a graphic representation of PHA activation of PBMCs from patient SB004 showing that HCV is not in T cells.

FIG. 20 shows the detection of HCV (E2) on Daudi cells upon co-cultivation with infected PBMCs in accordance with the present invention. Of note, Daudi cells are a B cell line.

FIG. 21 shows a comparison of different activation treatments (PBMCs from donor MLL-010). T1=PHA+IL-2. T2=SAC,+IL-2, and T3-T1+T2.

FIG. 22 is a histographic representation of viral RNA detection in cell supernatant (real time RT-PCR); of cells exposed to different treatments: T1, T2, T3 are the same as for FIG. 21. Of note, further addition of IL-4 to T3 further increased activation (data not shown).

FIG. 23 shows that HCV(+) and (−) strand RNA is produced de novo in activated PBLs. A) HCV-RNA was detected in PBLs from an HCV positive donor by a one step reverse transcription-polymerase-chain reaction (RT-PCR) followed by a nested PCR amplification using primers that targeted the highly conserved 5′ untranslated region (on-line material and methods). Total RNA, from either activated (P) or non-activated (N) cells, were prepared at the indicated times. RNA from Huh7 cells stably expressing the HCV replicon (Huh-Rep) (47) was used as positive control. RNA extracted from PBLs from an HCV negative donor and yeast tRNA were used as negative controls. B) Kinetics of HCV-RNA synthesis. PBLs from two positive donors, MLL-038 (Δ) and MLL-039 (O), were stimulated by method P. RNA was extracted at the indicated time of culture and the level of HCV(−) strand RNA was determined using the Roche LightCycler™ system. RNA levels were normalized against GAPDH and are reported as a fold variation relative to the amount of (−) strand RNA in non-treated PBLs. C), D) Bromo-uridine incorporation into de novo synthesized RNA was detected in by immunofluorescence using an anti-bromodeoxyuridine antibody, in which in C) HCV positive donor MLL-069, and in D) HCV negative donor.

FIG. 24 shows that HCV proteins are produced in activated PBLs. PBLs were stimulated using method P. Protein extracts were prepared following five days of activation. A) Extracts from either treated (P) or non-treated (N) PBLs, from donor SB-1 were run side by side with extracts from Huh-7 cells expressing the HCV replicon (Huh-Rep) (47). NS3 was detected using a polyclonal antibody). Extracts from PBLs, either treated (P) or non-treated (N), from a HCV negative donor were run side by side with extracts from donor SB6. NS3 was detected using monoclonal antibody 1G3D2. C) Extracts from Huh-7 cells and Huh-Rep, were run side by side with extracts, either treated (P) or non-treated (N), from an HCV negative and positive donor. NS5B was detected using a monoclonal antibody such as 5B-10 (IFA). D) Extracts from either treated (P or A) or non-treated (N) PBLs from different HCV positive donors were run side by side with extracts from an HCV negative donor, Huh-7 or Huh-Rep cells. NS3 was detected using monoclonal antibody 1G3D2. E) Kinetics of NS3 synthesis following PBLC stimulation by methods P, S and PS. Extracts were prepared on the indicated days and NS3 was detected using monoclonal antibody IG3D2. F, G, H) Kinetics of NS3 accumulation in donors MLL-001, MLL-002 and MLL-010 after stimulation using method P. Extracts were prepared on the indicated days. An extracts from non-treated cells was prepared either on day 3 (F and G) or on day 2 (H). NS3 was detected using anti-NS3 monoclonal antibody 1G3D2 (F and G) or with an NS3 rabbit antiserum (H). Actin or a non-specific band, LC, identified by antibody 1 G3D2, were used as loading controls. I, J, K.) siRNA silencing of HCV RNA. Core-siRNA or a non-specific RNA sequence (nsRNA) were electroporated into PBLs three days after stimulation. Proteins and RNA were extracted 48 hr later. I) NS3 and NS5B were detected with NS3 rabbit antiserum and 5B-3B1 monoclonal antibody (48), respectively. Actin was used as an internal control. J) RNA levels were quantified by real-time PCR (method 1, materials and methods). Absolute copy number of the HCV(+) strand transcripts (Δ) and the amount of GAPDH (O) RNA are shown. K) HCV RNA amounts were normalized against GAPDH. The ratio of HCV/GAPDH was determined for the nsRNA and assigned an arbitrary value of 100. The Core-siRNA HCV/GAPDH ratios are expressed relative to the negative control.

FIG. 25 shows that HCV Core protein was detected by indirect immunofluorescence in day 3 stimulated (P) PBLs from MLL-059, using the RR8 polyclonal antibody. Stimulated PBLs from an HCV negative donor were used as a control.

FIG. 26 shows that HCV is released from activated HCV positive PBLs. A, B) Supernatant from stimulated PBLs (method P) was collected and sedimented through a 20% sucrose cushion. A) Sedimented proteins were resolved by SDS 15%-PAGE, transferred to a nitrocellulose membrane (overnight, 30V) and detected using MAB255P monoclonal anti-core antibody (Maine Biotechnology Services, Inc.). HCV(−) corresponds to the negative control. B) RNA was analyzed by nested RT-PCR. RNA from Huh-Rep was used as a positive control. RNA from yeast tRNA, Huh-7, and an HCV negative donor were used as negative controls. C) PBLs from donor SB-5 were stimulated using methods B, P, and PS. Five days following activation, the supernatant was collected and sedimented through a 20% sucrose cushion. The quantity of HCV RNA was determined by real-time RT-PCR on the ABI Prism 7700 Sequence Detection System. D) Following metabolic labeling (35S Met/Cys) of PBLs from donor MLL-035, the supernatant was sedimented through a 20% sucrose cushion. The sediment was resuspended and analyzed by a flotation gradient. Collected fractions were resolved on a SDS-15% PAGE, transferred to a nitrocellulose membrane and exposed to a Kodak Biomax™ MR film. E) Fractions were concentrated and HCV E2 glycoprotein visualized by Western bolting using monoclonal anti-E2 1864 (450-470M) antibody. F) RNA was extracted from the gradient fractions of FIG. 26D. and the absolute quantity of HCV RNA was determined by real time RT-PCR. G) Fractions 1-4 (L) and 5-11 (H) from the flotation gradient were concentrated and pooled. Proteins were resolved on a SDS-15% PAGE. HCV E2 glycoprotein was detected using monoclonal antibody 1864 (450-470AA). Core protein was visualized using monoclonal anti-core 515S (20-40M) antibody. H) Activated PBLs from donors MLL-059 and MLL-064 were metabolic labeled for 12h with ³⁵S-Met/Cys or 32P-orthophosphate. Supernatants were sedimented through a 20% sucrose cushion. The sediments were resuspended and analyzed by a flotation gradient. The amount of incorporated radioactivity in each fraction of the gradients was determined in a Beckman LS 6500 scintillation counter.

FIG. 27 shows that virus released from activated HCV positive PBLs is infectious. A) Schematic representation of the co-culture chambers embodiment used in these experiments. B) MT4 cells were co-cultured with either treated (P) or non-treated (N) MTA cells, PBLs from two HCV negative donors or PBLs from donors SB-2 or SB-7. Extracts were prepared following six days of co-culture. NS3 was detected using monoclonal anti-NS3 antibody 1G3D2. LC indicates a non-specific band used as a loading control.

FIG. 28 shows Bromo-uridine incorporation into de novo synthesized RNA and detected by immunofluorescence using an anti-bromodeoxyuridine antibody in PBLs from donor MLL-065.

FIG. 29 shows a more detailed view of the HCV replication cycle as compared to that of FIG. 2.

FIG. 30 A shows one protocol to detect HCV RNA in PBLs using a plasmid vector comprising an HCV sequence downstream from a cassette comprising a hairpin structure-comprising a 5′ end, and a schematic representation of a method to amplify and detect HCV RNA.

FIG. 30B shows the detection of HCV protein by immunoprecipitation.

FIG. 31 shows the detection of HCV protein by Western Blot (A being activated).

FIG. 32 shows immunofluorescence of HCV(−) Control Polyclonal-anti Core RR8.

FIG. 33 shows immunofluorescence of MLL-059 Anti-Core RR8.

FIG. 34 shows immunofluorescence of MLL-059 Anti-Core RR8.

FIG. 35 shows immunofluorescence of MLL-059 Anti-Core RR8.

FIG. 36 shows immuno-electronmicroscopy of HCV protein using an anti NS3 antibody.

FIG. 37 shows electron microscopy of cells showing HCV viral particle assembly.

FIG. 38 is a schematic representation of one embodiment of a scheme for virus partial purification.

FIG. 39 shows density determination of HCV viral particles purified according to FIG. 38.

FIG. 40 shows by Western blotting that PBMC generate two HCV subpopulations that can be partially purified by density gradient.

FIG. 41 is a schematic representation of an embodiment of a protocol to assess infectivity of isolated HCV.

FIG. 42: shows that EBV-transformed B-Cell lines (#1-#6) express HCV proteins when stimulated.

FIG. 43 shows HCV(−) PBLs are infected with HCV when co-cultured with stimulated HCV(+) B-cell lines.

FIG. 44 is a schematic representation mechanisms that could explain the HCV-activation results described in the present invention, as involving the internal ribosome entry site (IRES).

FIG. 45 shows crosslinking to the HCV IRES.

FIG. 46 shows PBMCs Activation and HCV IRES Crosslinking pattern.

FIG. 47 shows crosslinking competition to the HCV IRES.

FIG. 48 shows crosslinking competition to the HCV IRES.

FIG. 49 shows crosslinking competition to the HCV IRES.

FIG. 50 is a schematic representation an overview of two HCV culturing systems of the present invention: the method for inducing hepatitis C virus (HCV) replication from peripheral mononuclear cells purified from chronically infected HCV donors; and the transformation of the B-cells using Epstein Barr virus (EBV) for immortalization of the B-cells. Further selection of single cell clones and the characterization of the virus harbored within is also shown on the right side.

FIG. 51 shows steps taken to generate EBV-transformed mixed B-cells populations (infected and non-infected).

FIG. 52 shows the detection of HCV positive strand RNA in EBV-transformed B-cell lines by real-time RT-PCR.

FIG. 53 shows the detection of HCV-Core protein in EBV-transformed B-cell lines. A) Negative control, EBV-transformed cells from an HCV negative donor. B) HCV core protein was detected by immunofluorescence in EBV-transformed B-cell line (EBV-2). The immunofluorescence patterns using the EBV-transformed cell lines can be compared to the preceding immunofluorescence figures with non-immortalized HCV producing cells.

FIG. 54 is a schematic representation of a method for generating single cell EBV-transformed B-cells populations. Single cell clones were generated by several rounds of limiting dilutions. HCV positive clones were screened by real time RT-PCR.

FIG. 55 shows a characterization of EBV-transformed single cell clones. A) Single cell positive clones were screened by RT-PCR thus, no nested PCR was used. Positive clones were further sequenced. B) Sequence of clone EBV-1.6 aligned with the infectious HCV-H77-C clone (provided by the NIH) used in these experiments as a positive control. The sequence of the virus found in the serum of donor MLL-005 from which the cell line was generated in indicated in blue. c) Sequence of clone EBV-2.8 aligned with the infectious HCV-H77-C clone (provided by the NIH).

FIG. 56 shows the propagation of HCV in naive T and B-cell lines. HCV was propagated in naive T cells (MT-4), non-EBV transformed B-cell lines (BJAB) and two EBV-transformed B-cell lines generated from HCV negative donors. HCV positive EBV-transformed cells were diluted (1/10) in naive cells giving origin to dilution 1. Three to four days (depending on cell confluence) after cells from dilution 1 were diluted (1/10) in naive cells, dilution 2. This process was repeated for three weeks followed by RNA analysis. Virus RNA was detected by RT-PCR. Cells generated by dilution of clone EBV-9.2 were further characterized.

FIG. 57 shows a sequence analysis of cell lines generated by dilution of clone EBV-9.2 in naive cells. A, B) Sequences generated from the different cell lined are aligned and compared with the original clone EBV-9.2. Nucleotides that vary during propagation are indicated in red. c) Mutations generated while diluting clone EBV-9.2 in BJAB a non EBV-transformed B-cell line are incorporated in the known structure of the HCV IRES. Red arrows indicate the beginning of the sense and antisense RT-PCR primers that determine the amplified region. Nucleotides in Red indicate the variation of clone EBV-9.2 when diluted in BJAB. Blue nucleotides (also italicized and underlined) and arrows indicate mutations generated while diluting clone EBV-9.2 in MT4 and in naive EBV-transformed B-cell lines.

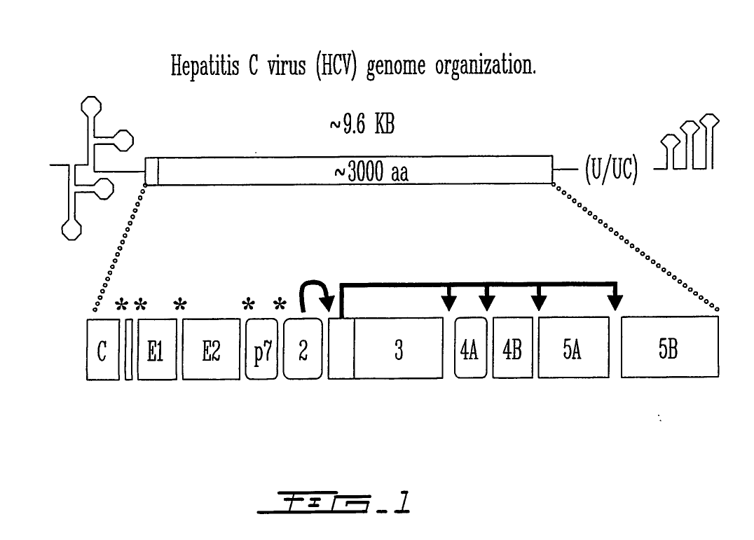

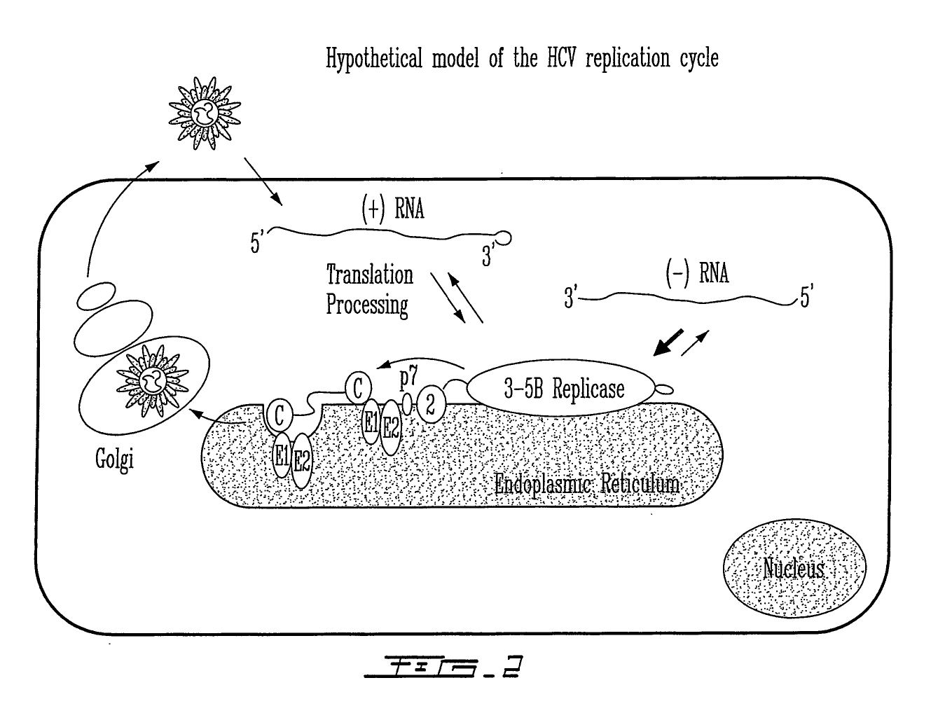

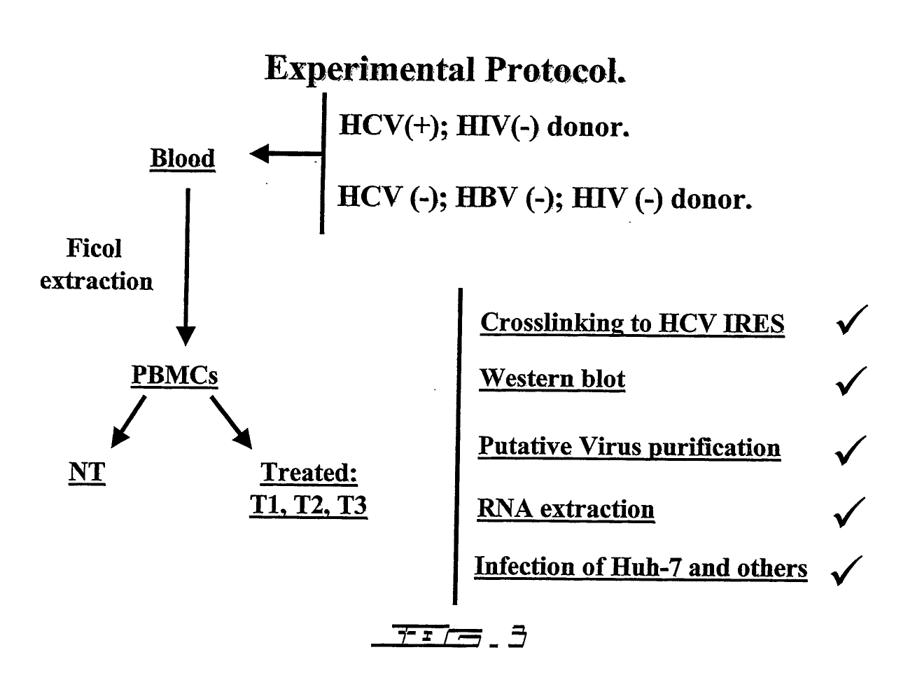

Other objects, advantages and features of the present invention will become more apparent upon reading of the following non-restrictive description of preferred embodiments with reference to the accompanying drawings which is exemplary and should not be interpreted as limiting the scope of the present invention.

### DESCRIPTION OF THE PREFERRED EMBODIMENTS

The existence of extrahepatic reservoirs of hepatitis C virus (HCV) replication remains controversial. Several groups have described the presence of hepatitis C virus (HCV) genomic sequences (plus-strand) and replicative. intermediate (minus-strand) in peripheral blood mononuclear cells (PBMC). The association of HCV RNA with peripheral blood leukocytes has been documented since 1992 However, the specificity of the methods used in these studies has been questioned. More recent reports, which used an optimized negative strand-specific reverse-transcriptase polymerase chain reaction (RT-PCR) assay, detected negative-strand HCV only in PBMC taken from post-transplant or human immunodeficiency virus (HIV)-coinfected HCV patients, and not in PBMC from typical patients with chronic HCV infection. Of note, a number of studies have also reported that human B and T cell lines are capable of supporting a productive infection. However, the data supporting viral production was only based on RNA detection (Proc. Natl. Acad. Sci. USA, 1992, 89:5477; J Virol. 1993, 67:1953; Ibid, 1996, 70:3325-9; Ibid, 70:7219-23; Hepatology 1996, 23:205; Antiviral Research 2001, 52:1-17). The validity of these data have been questioned (Laskus et al. 1998, see below). Moreover, PBMC obtained from HCV negative donors were successfully infected using HCV-positive sera, demonstrating that PBMCs are permissive for HCV replication in vitro (J. Gen. Virol. 1995, 76:2485-2491). However, replication of the virus therein was really low. In addition, only RNA was detected. Thus, prior to the present invention, it remained unclear whether HCV could actively replicate to workable levels in PBMCs.

Using an immunodeficiency (SCID) mouse model that allows long-term survival of human hematopoietic cells, Bronowicki et al. (1998) presented strong evidence for persistence of HCV RNA in PBMCs obtained from HCV positive donors (Hepatology 1998, 28:211-218). The susceptibility of PBMC to HCV infection has been corroborated by in situ hybridization techniques showing both positive and negative polarity RNA strands in circulating and/or bone marrow recruited mononuclear cells. Recent reports have established that HCV is in fact associated to B cells. Based on the model of Epstein-Barr virus another B-cell-tropic virus, that remains latent while the host cell is quiescent but is reactivated and enters a lytic replication phase once the host cell is activated (J. Virol. Methods, 1988, 21:223-227; Annual Rev. Microbiol. 2000, 54:19-48), Boisvert et al., (2001) examined the possibility that HCV could replicate in peripheral B cells, but under altered physiological conditions, such as immunosupression or cellular activation. The authors could not detect HCV replication in enriched B cells obtained from HCV positive donors upon cell stimulation with CD40L.

Considering the observations of Laskus et al., (1998) showing the presence of active HCV replication in lymphoid tissue in patients coinfected with HIV (not in non-HIV infected patients), suggesting that co-infection of HIV would be required in HCV cell-based assay, and those of Boisvert et al., (2001), we hypothesized that HCV replication in peripheral blood leukocytes (PBML) requires cell activation (e.g. in the mixture of the T-and B-cell population).

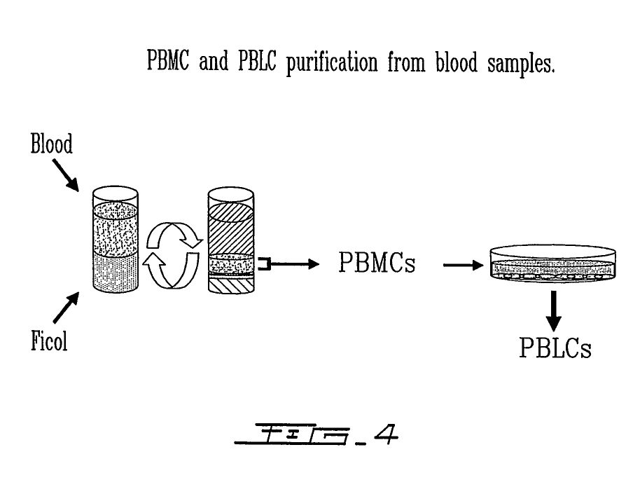

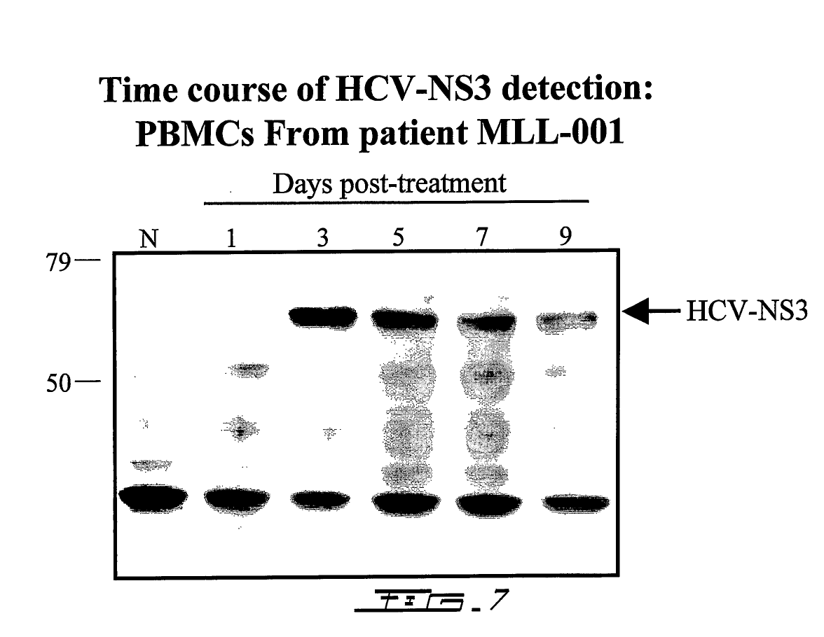

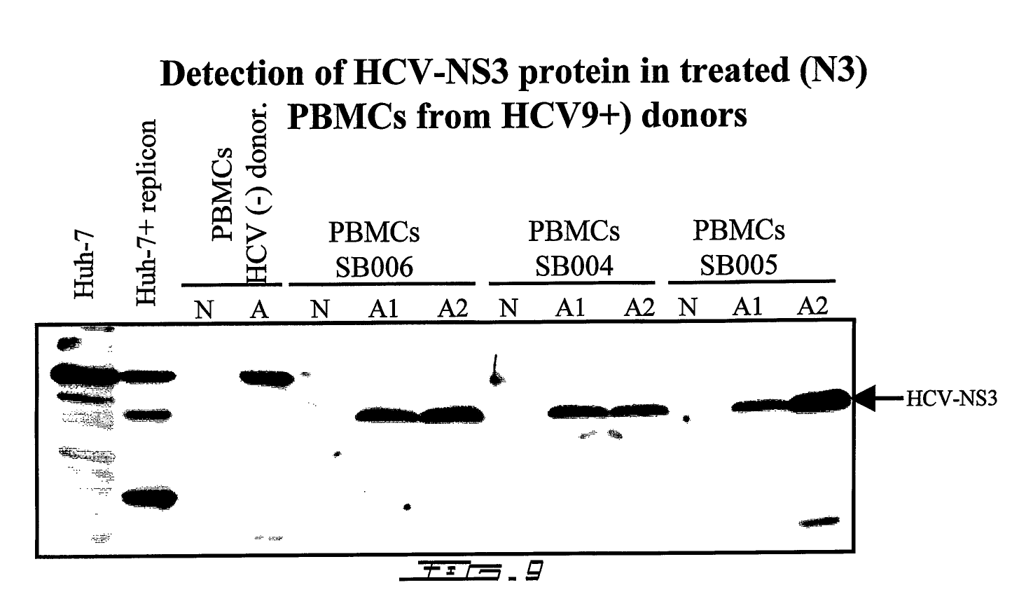

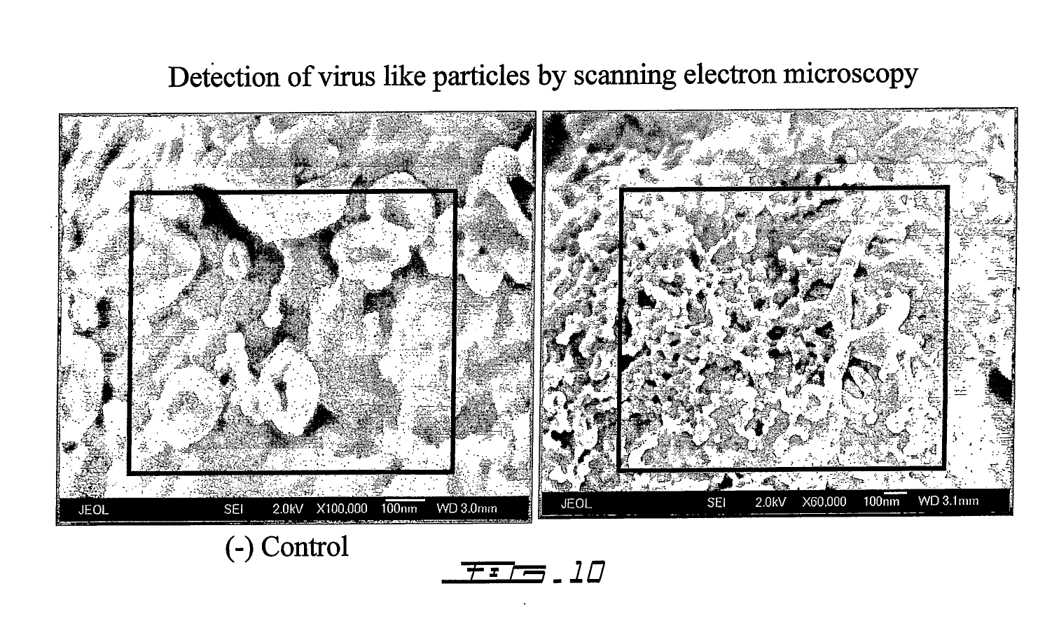

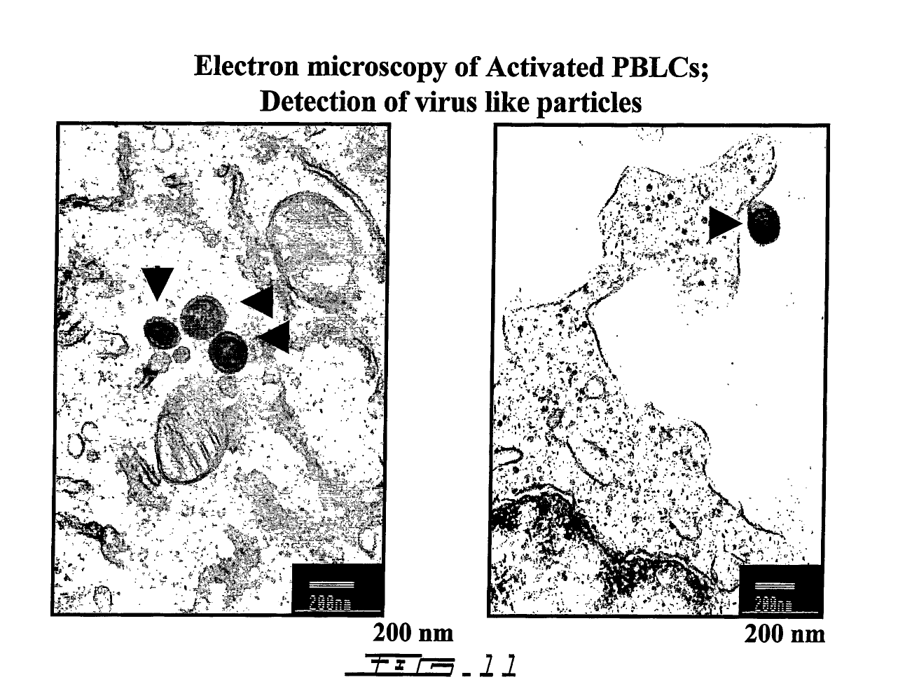

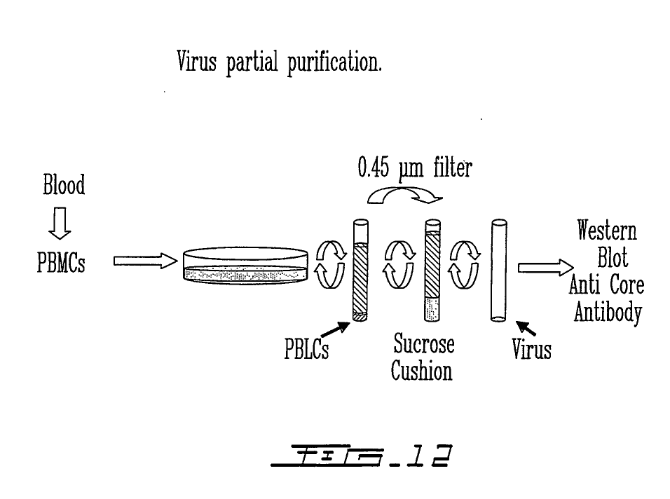

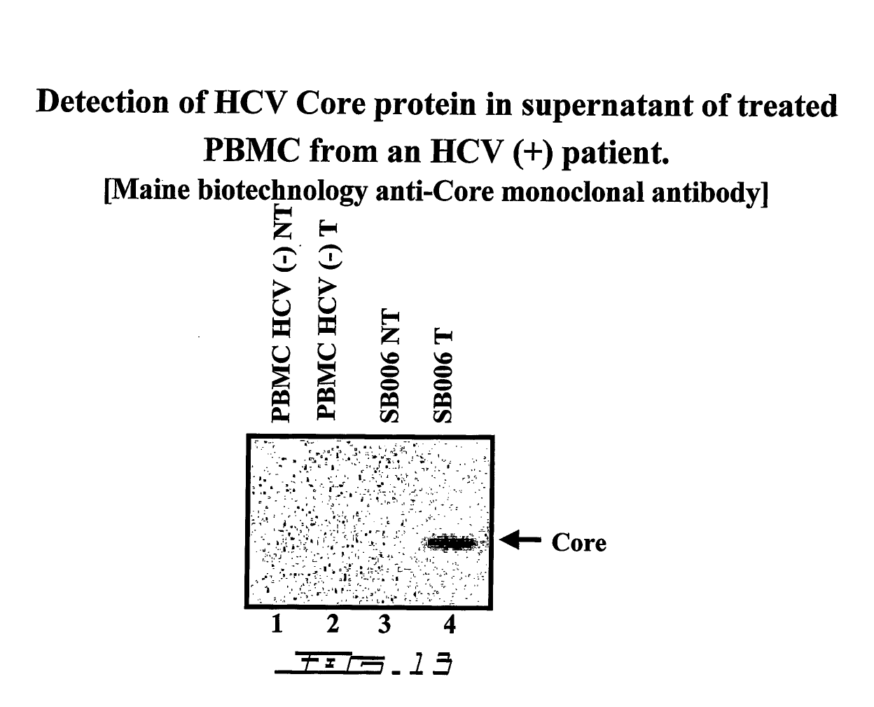

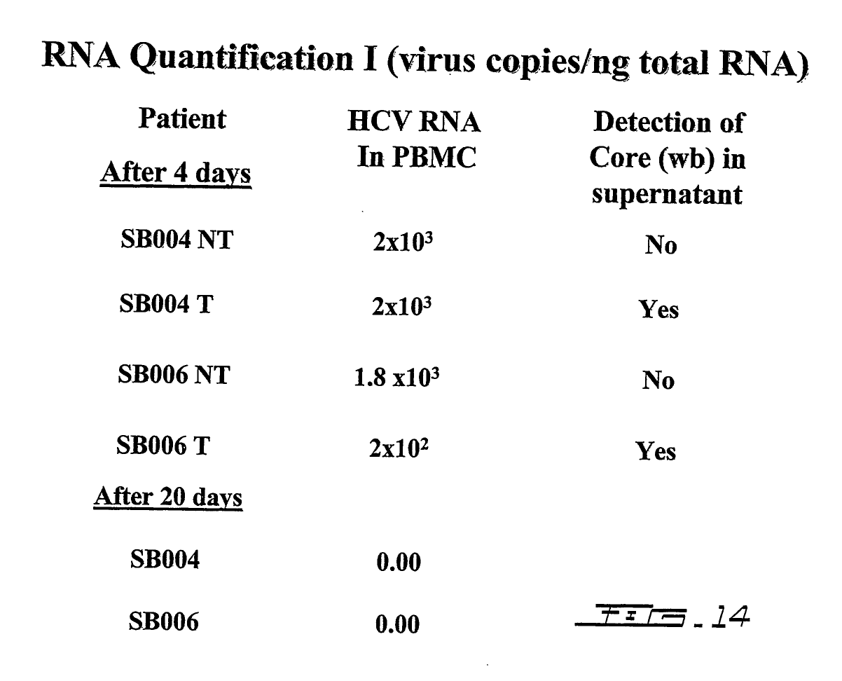

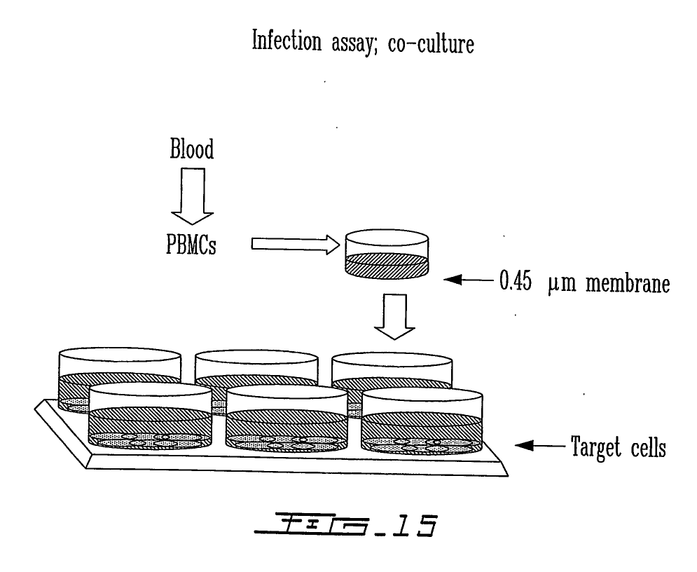

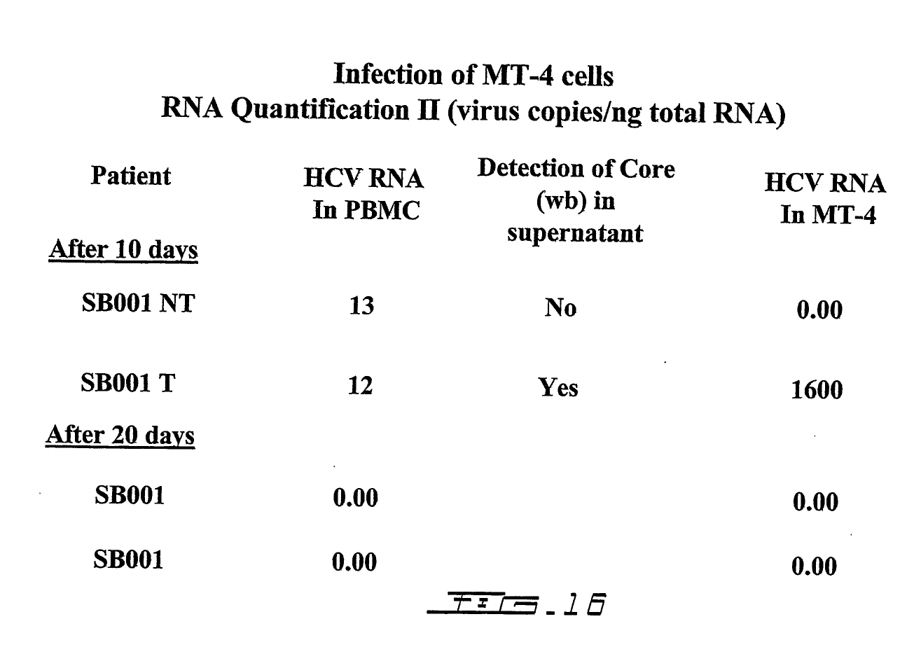

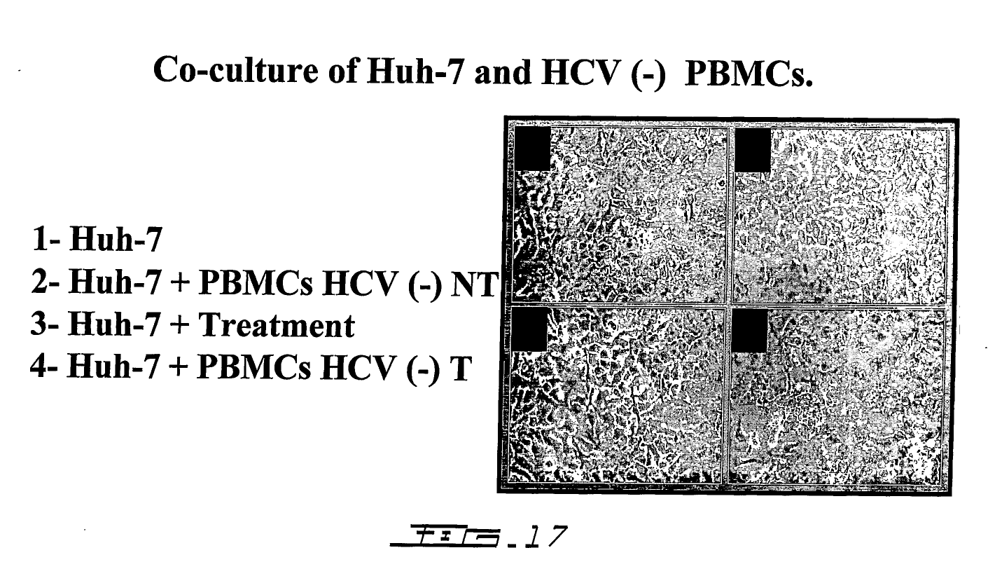

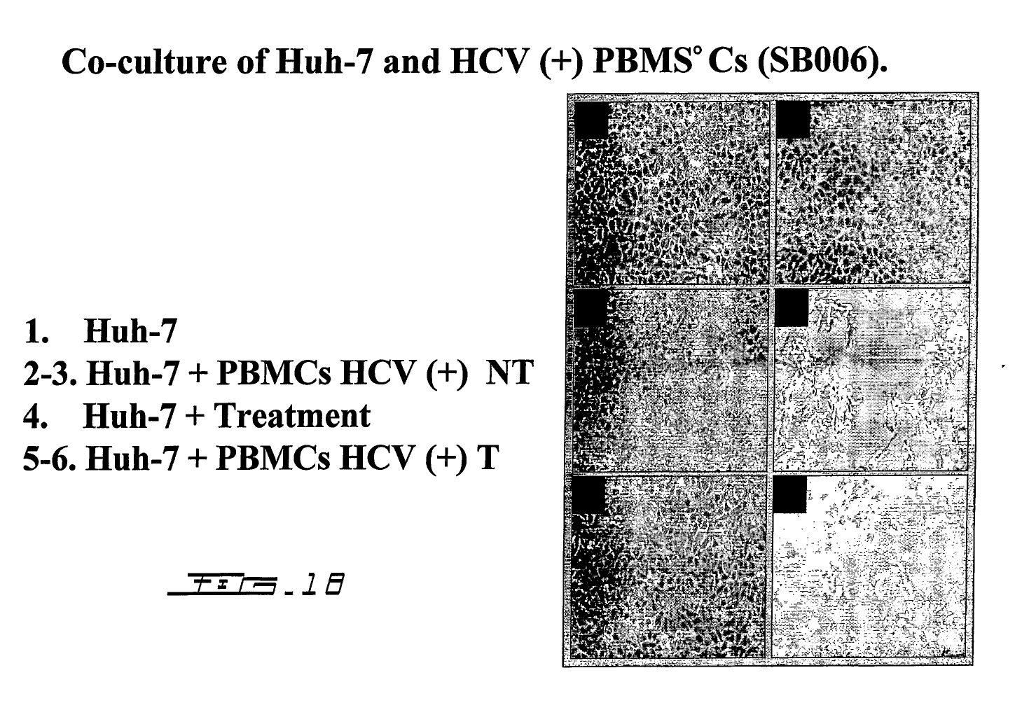

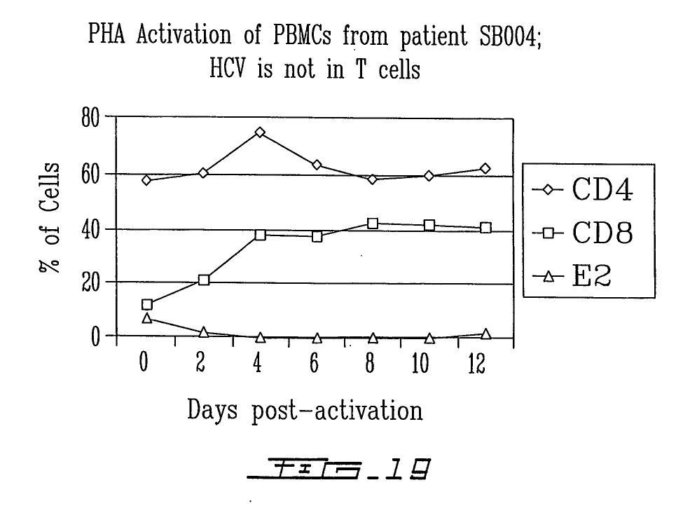

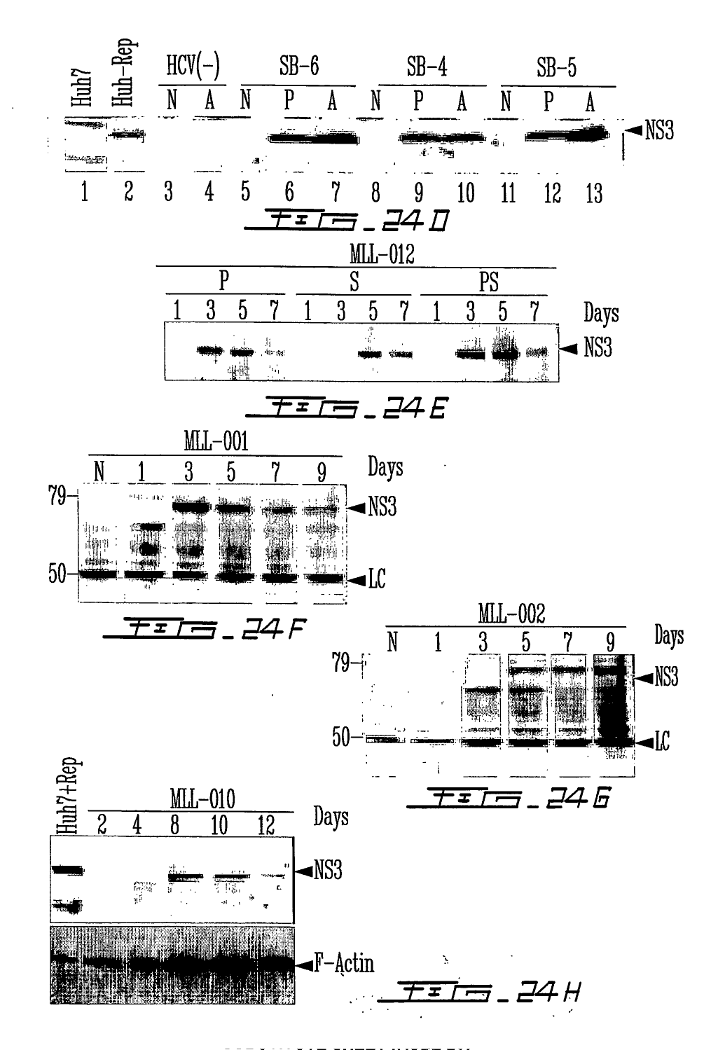

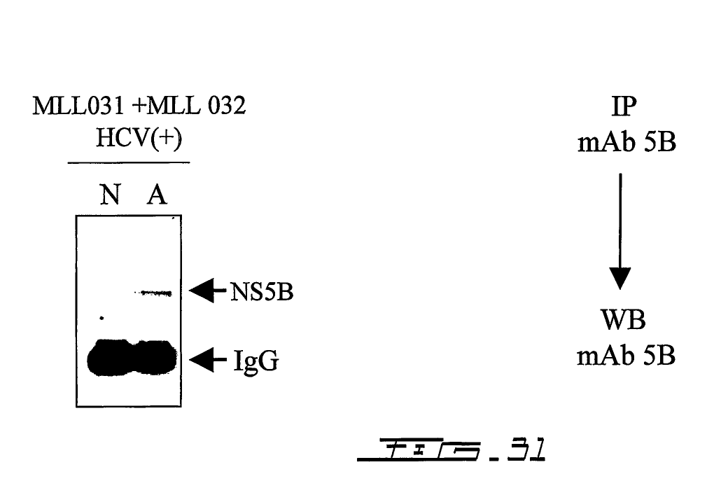

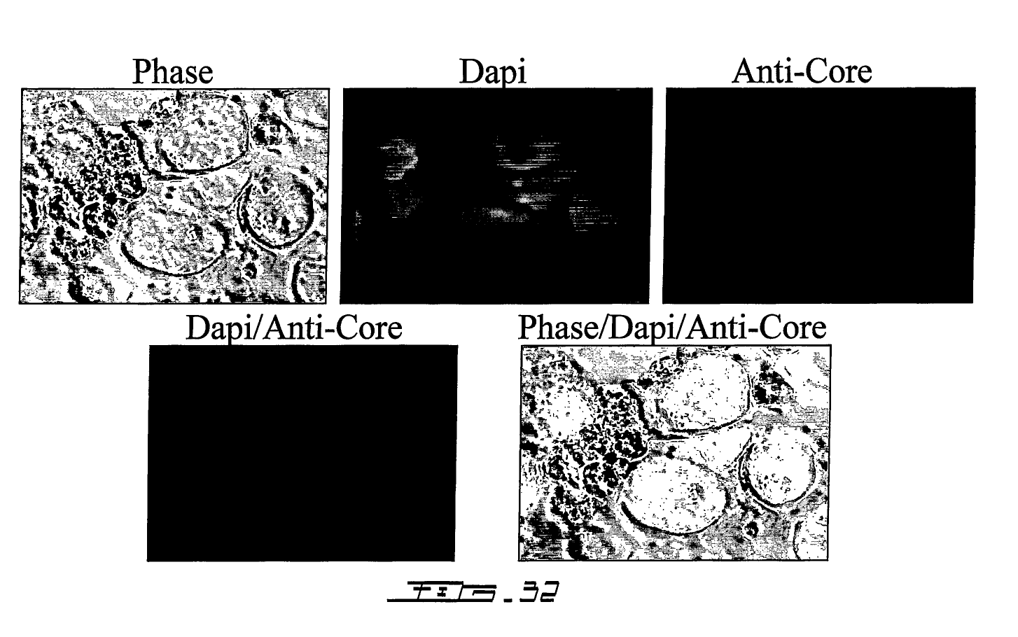

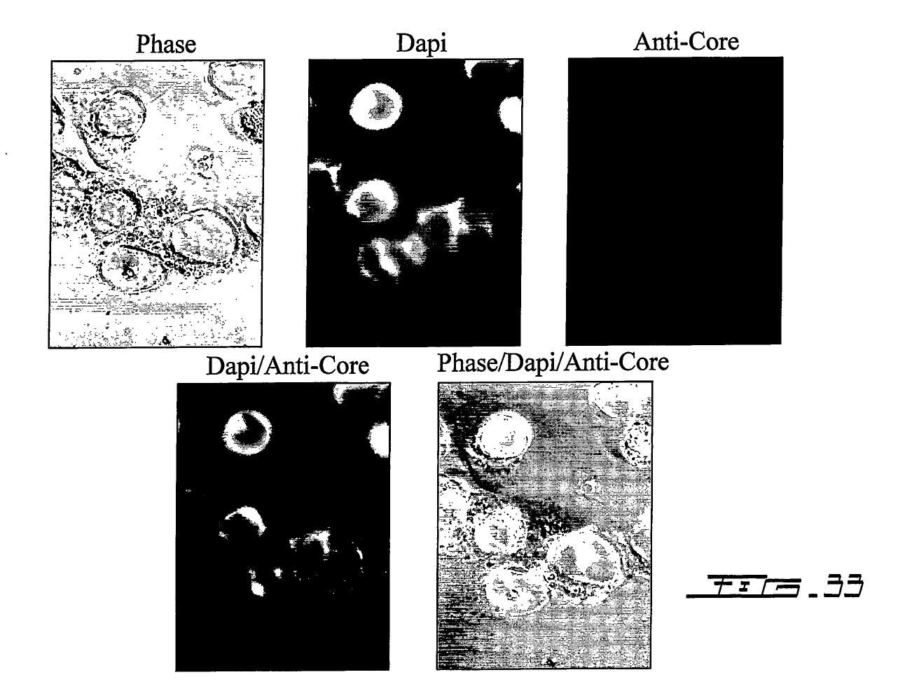

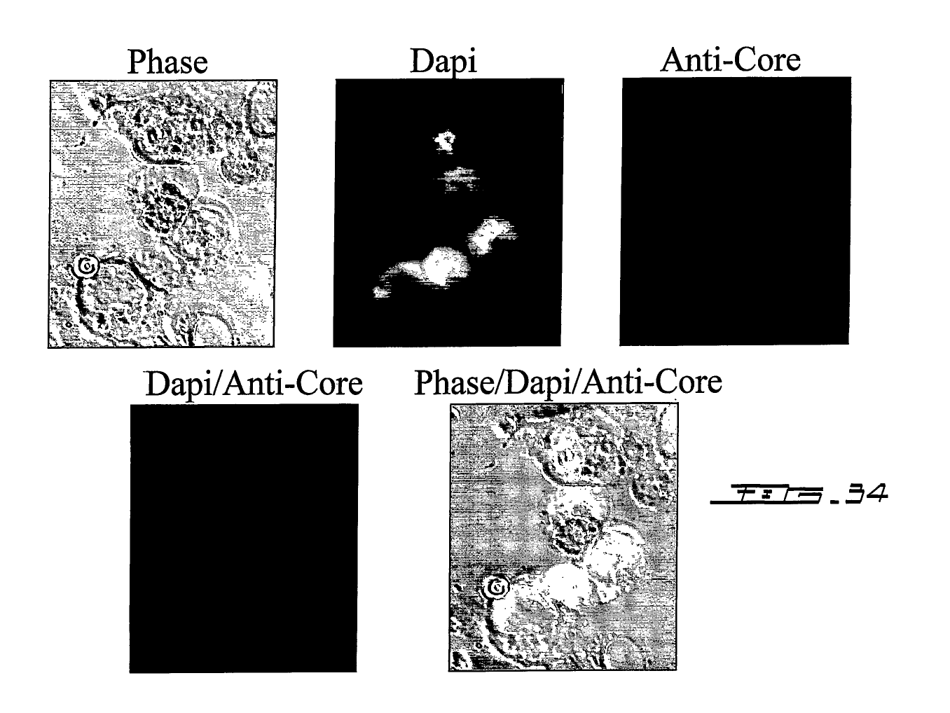

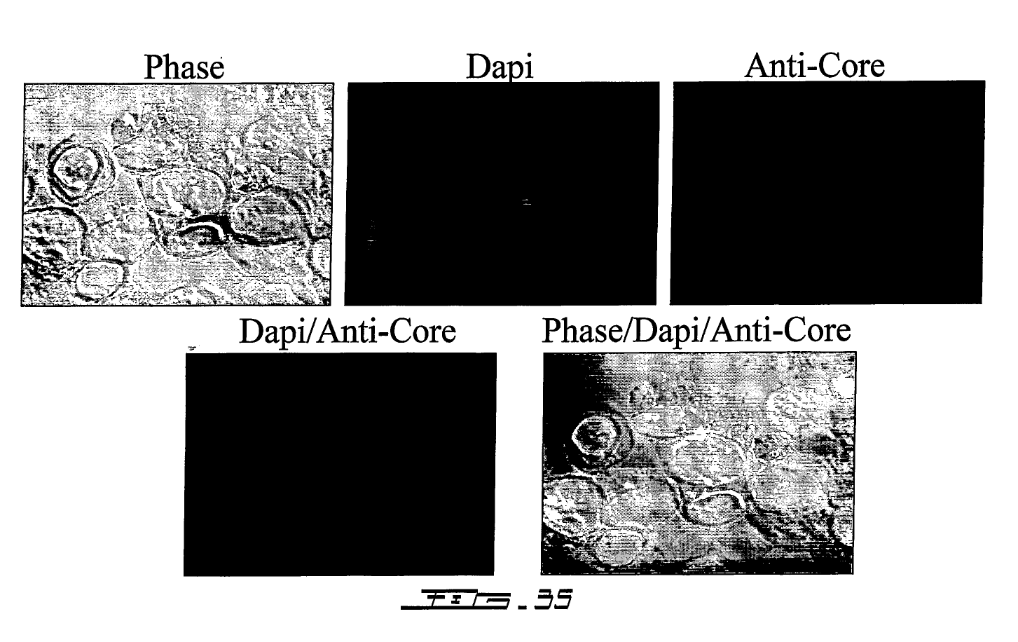

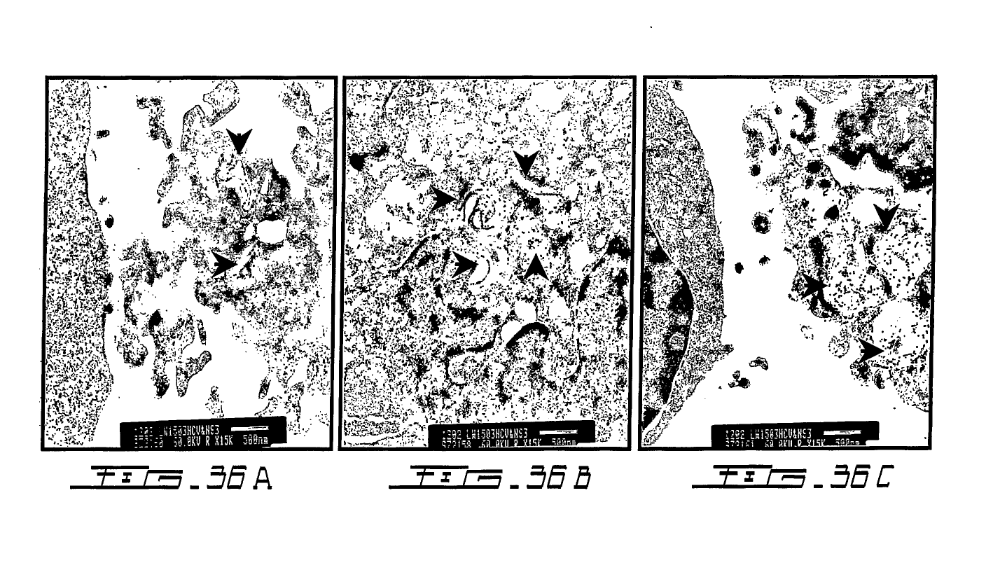

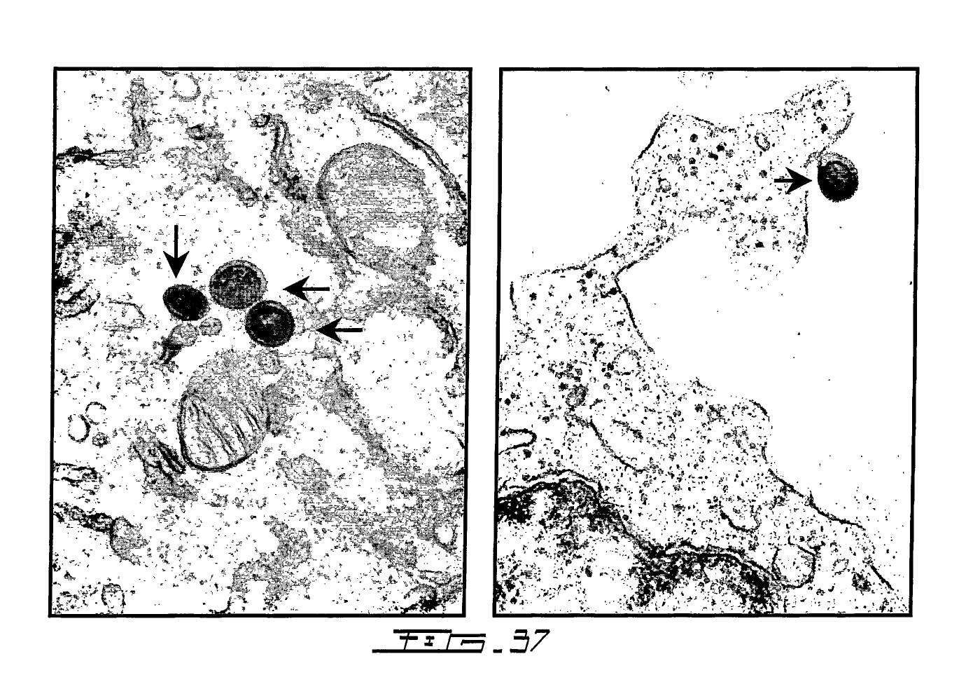

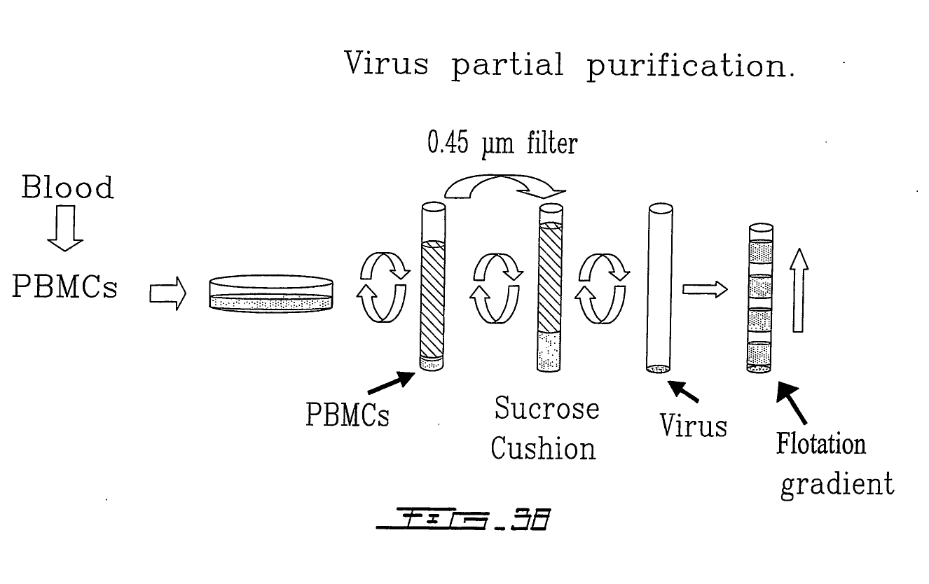

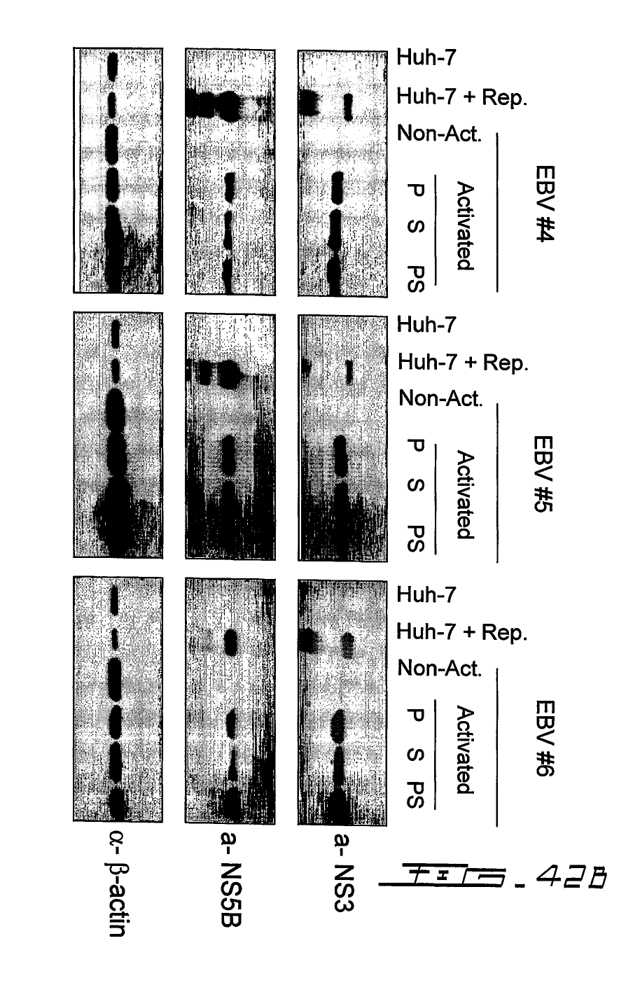

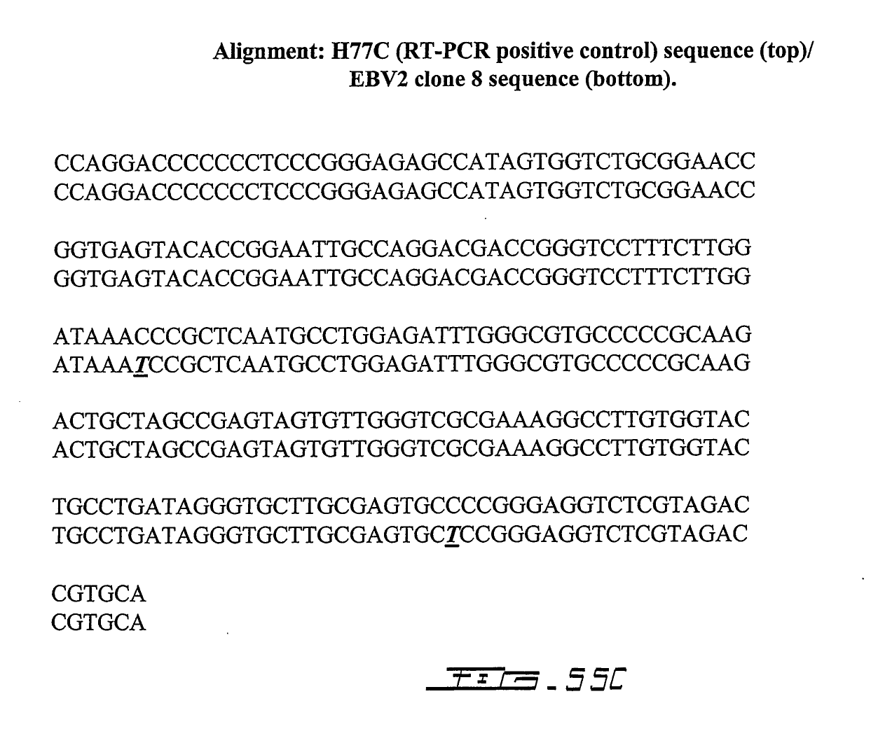

Until now, all studies of HCV replication have concentrated on documenting the presence of the replicative intermediate (minus-strand). RNA. However, the validity of these reports has been criticized because the presence of viral proteins was not demonstrated. It stands to reason that in order for replication to occur, protein expression is required. Therefore, in order to sustain the observations relating to activated PBMCs, it was important to show protein expression. Thus, non-structural (NS) HCV proteins were chosen as an indicator of viral replication (See FIGS. 3, 5-9, 13-16, 21, 24-27, 31-36, 40, 42-43 and 53). The studies presented hereinbelow clearly demonstrate that PBMCs obtained from HCV seropositive donors are able to support at least one complete cycle of viral replication upon activation. For this, a simple method that actively induces virus replication within the infected cell was developed.

Most circulating leukocytes are. in a resting state, but remain responsive to mitogenic signal that can induce cell activation. Lymphocyte activation in response to extrinsic signals results in either progression through the cell cycle, or activation of proapoptotic pathway(s) (Cell 1991, 65:921-923; Science 1996, 274:1664-1672). Lymphocyte activation correlates with a strong increase in translation rates and expression of translation initiation factors (J. Immunol. 1998, 160: 3269-3273). The change in the cellular environment associated with immune activation could induce HCV protein synthesis and initiate a cascade of events leading to an impaired cell cycle and an enhanced viral replication.

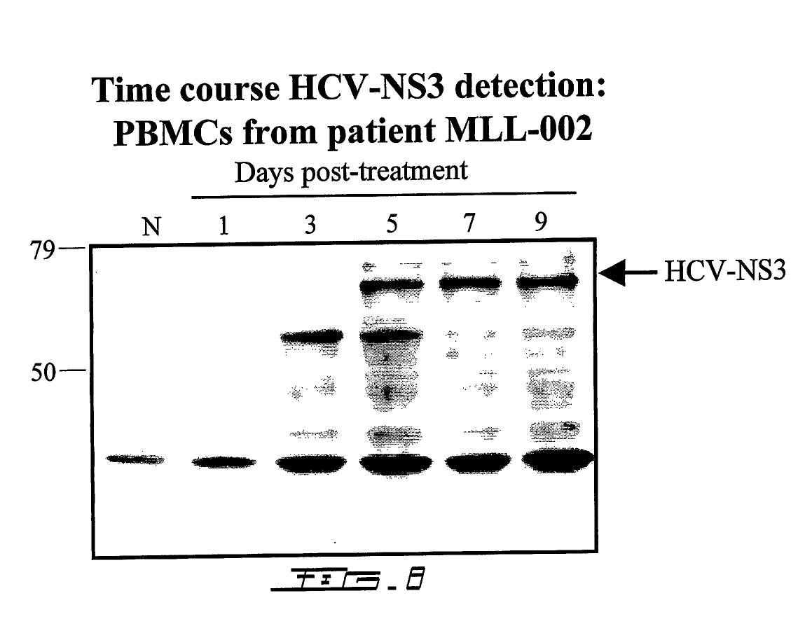

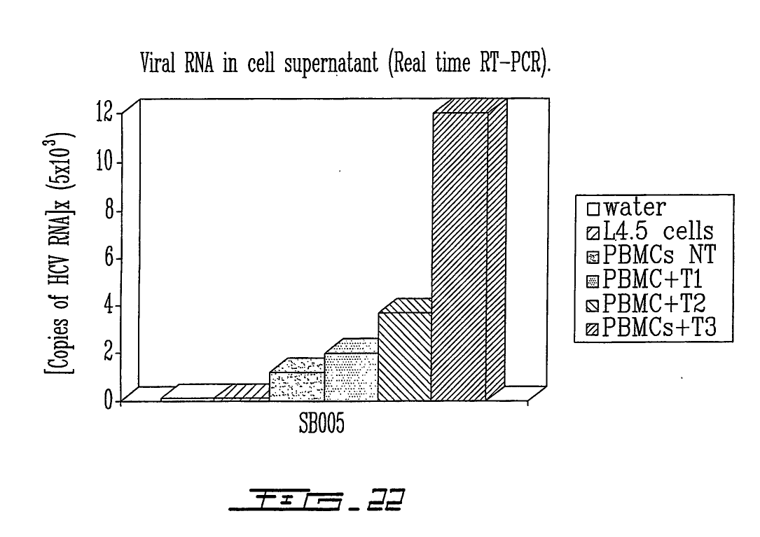

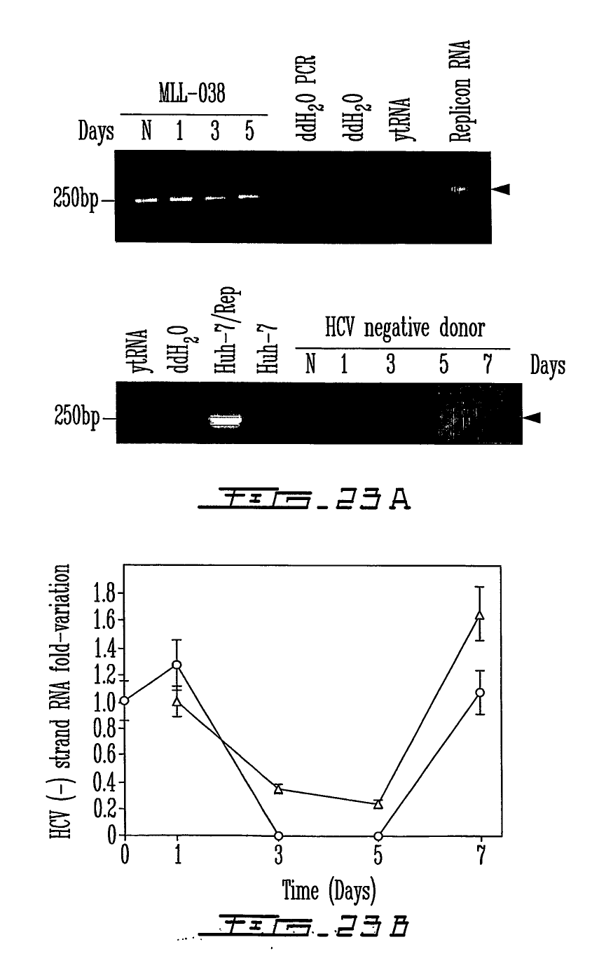

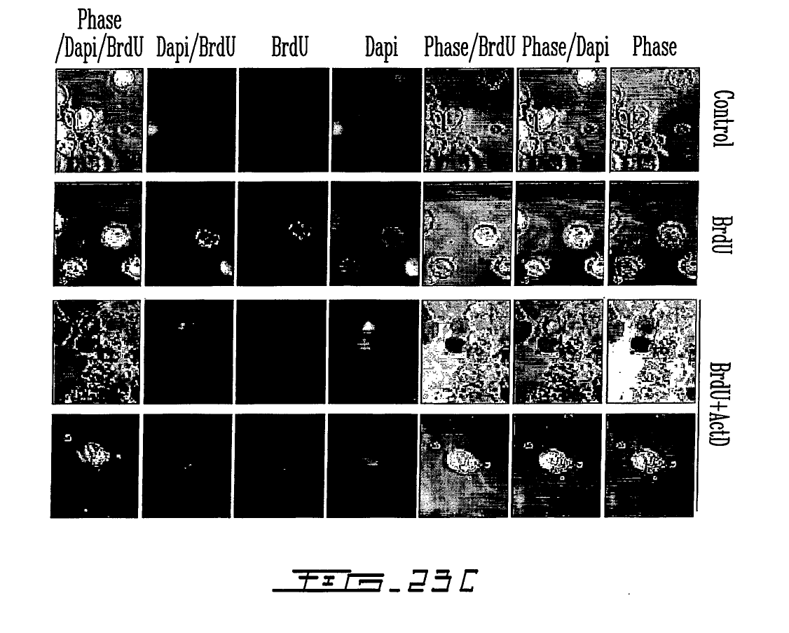

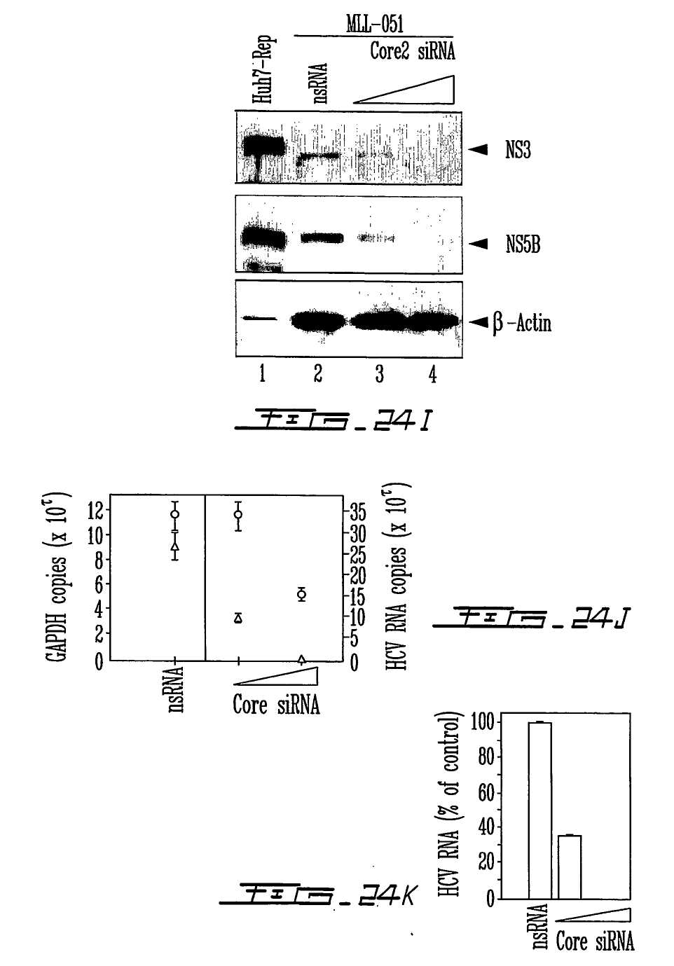

In accordance with the present invention, the activation of PBMCs (or PBLs) is achieved using at least one mitogenic (or activating agent). In one particular embodiment, the activating agent is a mixture of antigen-nonspecific T and/or B cell activators (Anti-CD3 antibody, phytohemagglutinin (PHA), CD40L, Staphylococcus aureus crown I (SAC), IL2 and IL4). Of course, it will be realized that other T and B cell activating agents exist and are well-known in the art. Such agents could be used in the methods and culture systems of the present invention. The result of activation upon HCV viral replication can be seen in FIGS. 6-9, 13-14 and 16-22.

In one particular embodiment, Ag-specific T and/or B cell activating agents could also be used. It will also be understood that the present invention provides assays which can be used to identify further activating agents, mixtures thereof or other nutrients which can further activate the HCV-producing cells of the present invention and/or promote a longer survival thereof in culture. For example, having shown that PBMCs or PBLs can be activated to replicate HCV, other inducers and mixtures thereof can be tested, and the HCV production or replication cycle monitored to identify other inducers or combination thereof (cheaper, more efficient, more adapted to specific strains or the like).

In accordance with one embodiment, HCV non-structural proteins (NS3 and NS5) were chosen and detected by Western blot analysis. Virus-like particles could be detected within the infected cells by electron microscopy demonstrating that viral proteins are assembling. Viral particles could be isolated from the PBMCs supernatant. The presence of virus was evidenced from Western blot (anti-Core) analysis and genomic RNA detection by real time RT-PCR, this observation shows that upon assembly, viral particles were actively being liberated to the supernatant.

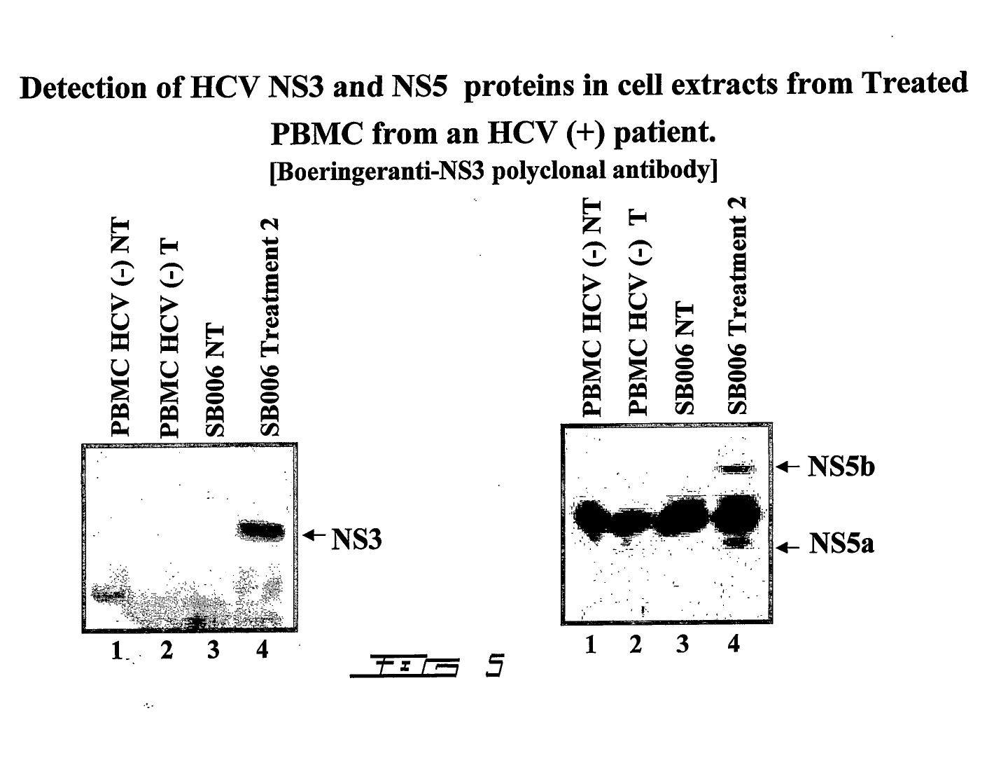

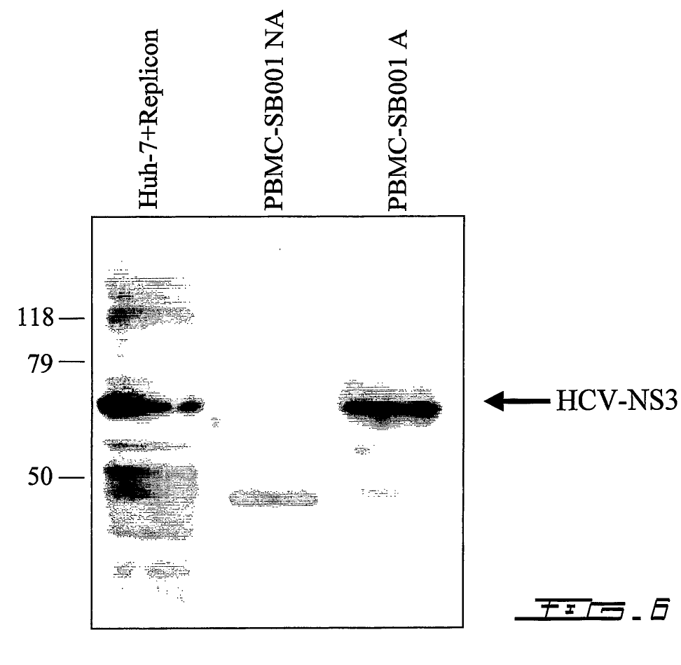

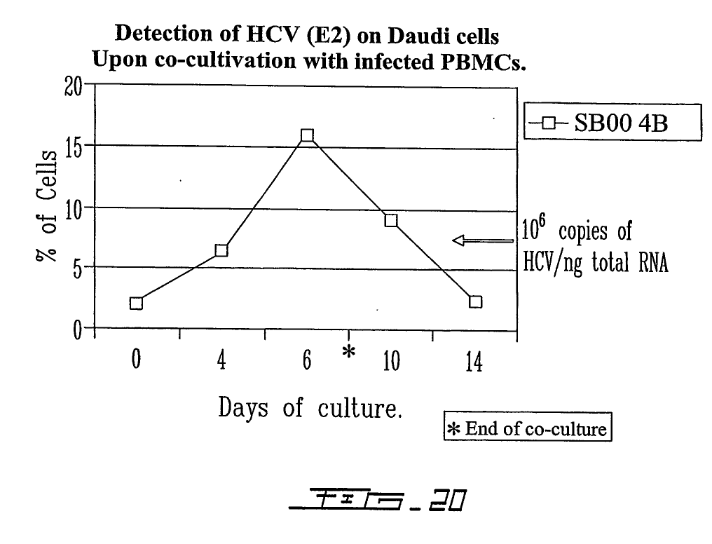

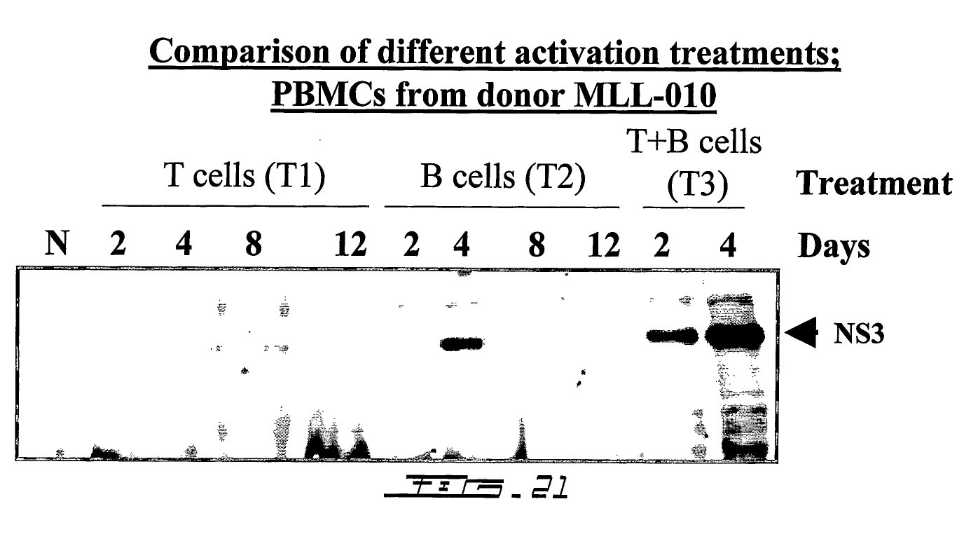

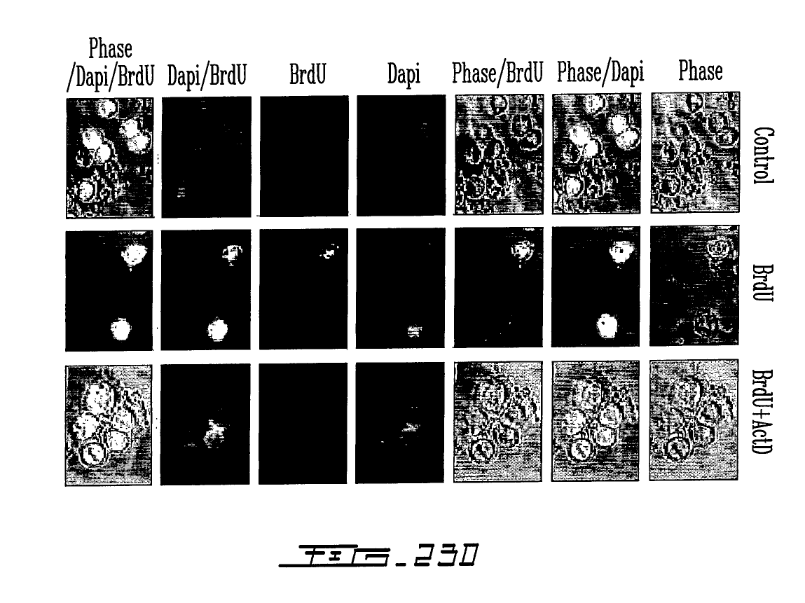

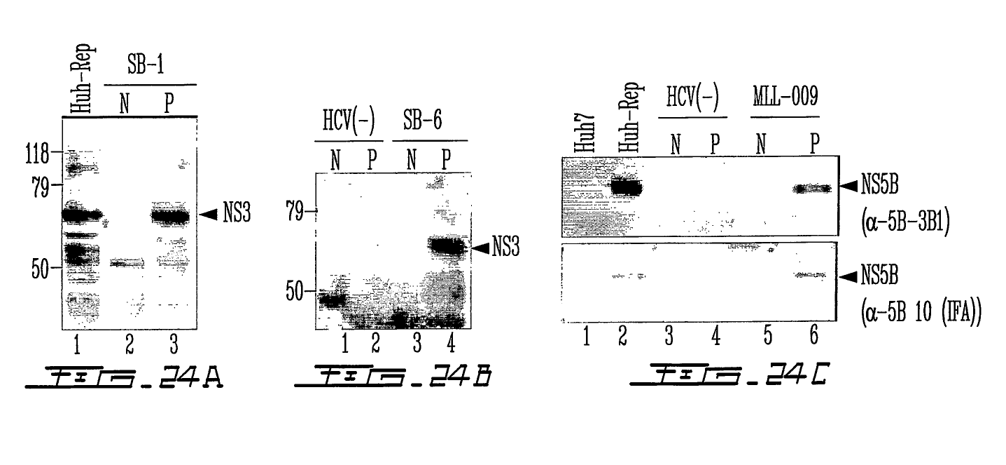

Moreover, using a co-culture method (see for example FIGS. 3, 4, 15 and 27) it was demonstrated that the HCV particles produced in PBMC could infect other cells (FIG. 3). Non-limiting examples thereof include liver cells such as Huh-7 (FIGS. 3, 15 and 17-18), Daudi (B-cell) (FIG. 20), MT4 (T-cell) cell lines (FIG. 16), naïve PBLs and thus B and T cell lines as well as primary lymphocytes. Thus, not only is it shown that HCV can replicate, and assemble in the tissue culture system of the present invention, but it is also shown that it can also infect other cells. Infection was monitored by detection of viral RNA (real time RT-PCR). The results generated by these experiments has a significant impact on the testing of anti-HCV agents. Of course, it also serves as a proof of principle that PBMC are able to sustain HCV infection and generate infective HCV. Moreover these data strongly suggest that both the serum and PBMCs obtained from HCV positive donors can be used as a source of infectious virus to infect naïve cells such as monocyte and/or monocyte-derived dendritic cells (DCs). Therefore, the instant invention, which enables the infection of cells with HCV, is by itself a significant achievement.

I. Synthesis of Nucleic Acid

Isolated nucleic acid molecules of the present invention are meant to include those that result from any known method, such as chemically synthesized. Similarly, an oligomer which corresponds generically to a HCV nucleic acid molecule, or to a strain specific HCV nucleic acid, can be synthesized. Such synthetic oligonucleotides can be prepared, for example, by the triester method of Matteucci et al, J. Am. Chem. Soc. 103:3185-3191 (1981) or by using an automated DNA synthesizer.

An oligonucleotide can be derived synthetically or by cloning. If necessary, the 5′-ends of the oligomers can be phosphorylated using T4 polynucleotide kinase. Kinasing of single strands prior to annealing or for labeling can be achieved using an excess of the enzyme. If kinasing is for the labeling of probe, the ATP can contain high specific activity radioisotopes. Then, the DNA oligomer can be subjected to annealing and ligation with T4 ligase or the like.

II. Specific Detection of HCV Nucleic Acid or Protein

The present invention relates in one embodiment, to a nucleic acid for the specific detection, in a sample, of the presence of HCV nucleic acid sequences.

In one preferred embodiment, the present invention relates to oligomers which specifically target and enable amplification (i.e. primers) of HCV RNA sequences associated with infection.

In one embodiment, the amplified product can be detected following hybridizing with a probe which consists of an isolated nucleic acid consisting of 10 to 1000 nucleotides (preferably, 10 to 500, 10 to 100, 10 to 50, 10 to 35, 20 to 1000, 20 to 500, 20 to 100, 20 to 50, or 20 to 35) which hybridizes preferentially to an amplified product which originated from HCV RNA, wherein said nucleic acid probe is or is complementary to a nucleotide sequence consisting of at least 10 consecutive nucleotides (preferably, 15, 18, 20, 25, or 30) from the known HCV polynucleotide sequence.

Amplification of chosen HCV sequences is exemplified herein. Of course, other sequences, primers and probes could be used in accordance with the present invention.

Primer in accordance with the present invention can be designed as commonly known in the art based on the sequences of HCV available publicly or of the sequences of HCV shown herein.

Of course, as will be understood by the person of ordinary skill, a multitude of additional probes or primers can be designed from the same or other region of HCV.

The hybridization probes of the present invention can be labeled by standard labeling techniques such as with a radiolabel, enzyme label, fluorescent label, biotin-avidin label, chemiluminescence, and the like. After hybridization, the probes can be visualized using known methods.

The nucleic acid probes of the present invention include RNA, as well as DNA probes, such probes being generated using techniques known in the art.

In one embodiment of the above described method, a nucleic acid probe is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The sample used in the above-described methods will vary based on the assay format, the detection method and the nature of the tissues, cells or extracts to be assayed. Methods for preparing nucleic acid extracts from cells, purifying cells and the like are well known in the art and can be readily adapted in order to obtain a sample which is compatible with the method utilized.

III. A Method of Detecting the Presence of HCV in a Sample

In another embodiment, the present invention relates to a method of detecting the presence of HCV nucleic acid in a sample comprising a) co-culturing a cell sample with a target cell under HCV-activating conditions, and b) detecting the presence of the HCV nucleic acid using an amplification method. One skilled in the art would select the nucleic acid primers according to techniques known in the art as described above.

In another embodiment, HCV protein is detected using a HCV-specific ligand, such as an antibody

IV. A Kit for Detecting the Presence of HCV in a Sample

In another embodiment, the present invention relates to a kit for detecting the presence of HCV in a sample comprising at least one container means having disposed therein at least one primer pair. In a preferred embodiment, the kit further comprises at least one further container comprising one or more of the following: amplification reagents, probes, wash reagents and reagents capable of detecting the presence of bound nucleic acid probe. Examples of detection reagents include, but are not limited to radiolabelled probes, enzymatic labeled probes (horse radish peroxidase, alkaline phosphatase), and affinity labeled probes (biotin, avidin, or steptavidin).

In detail, a compartmentalized kit includes any kit in which reagents are contained in separate containers. Such containers include small glass containers, plastic containers or strips of plastic or paper. Such containers allow the efficient transfer of reagents from. one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like.

One skilled in the art will readily recognize that the nucleic acid probes described in the present invention can readily be incorporated into one of the established kit formats which are well known in the art.

Of course, the kit can also be based on a detection of HCV protein as opposed to HCV nucleic acid. In one embodiment, a first container would contain an antibody specific to a HCV protein. In a particular embodiment, the kit is adapted to be used on HCV expressing cells obtained by the co-culturing system of the present invention, or by the immortalizing system of the present invention.

V. Diagnostic Screening

It is to be understood that although the following discussion is specifically directed to human patients, the teachings are also applicable to any animal that expresses HCV.

According to the invention, presymptomatic and post-symptomatic screening of an individual is now possible. This is especially valuable for the identification of non-symptomatic carriers of HCV. Early diagnosis is also desired to maximize appropriate timely intervention.

Probes that detect a chosen HCV sequence may be labeled with any of a variety of labels that can, directly or indirectly, result in a signal when the probe is hybridized to the amplified target sequence. For example, a label may be any moiety that produces a luminescent, fluorescent, radioactive, or enzymatic signal that can be detected by using methods well known in the art. A probe need not be labeled with a label moiety if binding of the probe specifically to the amplified nucleic acid containing the exon-exon junction results in a detectable signal, such as, for example a detectable electrical impulse.

VI. A Novel Tool for Developing a HCV Vaccine

Adoptive transfer of donor-derived virus-specific T cells generated in cultures with antigen-bearing autologous monocyte-derived dendritic cells (DCs) has attracted considerable attention as a promising tool to generate a strong immune response (Int. J. Cancer. 2001, 94:459-73; Exp. HematoL 2001, 29:1247-55; Trends Mol. Med. 2001, 7 :388-94). This technique has not only proved useful as an alternative anti-cancer strategy but also as a novel anti-virus therapy. For example, when DCs were pulsed with human cytomegalovirus virus (HCMV) antigen and cocultured with autologous peripheral blood lymphocytes from HCMV-seropositive individuals, there was an increase in the numbers of cytolytic T cells. This technique was used to enhance immunity in HCMV-seropositive transplant patients (Blood. 2000, 97: 994-1000).

Now having developed a technology to infect cells with HCV, it becomes possible to adapt the dendritic cells (DCs) technology mentioned above, to generate T-cell responses to HCV. Advantages for using DCs for this purpose include the fact that: i) they are considered the most potent of the antigen-presenting cells (APCs) (Blood. 1997, 90:32453287; Nature. 1998, 392:245-252); ii) their role in resistance against experimental malignancies and infections is well documented (J. Immunol. 1998, 161:2094-2098, J. Virol. 1998, 72:3812-3818); iii) DCs can be easily generated from bone marrow, cord blood, and peripheral blood; iv) DCs have the unique ability to process exogenously supplied antigen efficiently and present peptides on both class 1 and class 2 HLA molecules along with an array of costimulatory molecules (Nature. 1998, 392:245-252; Nature. 1999, 398:77-80); v) The presentation of both helper and CTL-defined epitopes suggests that both CD4+ and CD8+ HCV-specific T cells will be generated. This will allow both the generation of cytolytic effector function and the potential for re-establishment of longer-term immune memory, which may be important in preventing subsequent viral reactivation; and vi) The lack of an absolute knowledge of the presented peptides means that this technique can be used for patients of any HLA type and will trigger T-cell reactivity to undefined immunogenic determinants, thereby allowing a greater potential for augmentation of a broader T-cell response. It is thus expected that this will reduce the possibility that selective pressure will be applied to HCV in vivo. Based on the foregoing, it is predicted that the approach described herein (together with possible adaptations by a person of ordinary skill using the knowledge in the art) will contribute significantly to the design of a vaccine therapy towards HCV infection.

VII. Robust Hepatitis C Virus Replication in Peripheral Blood Lymphocytes from Infected Donors

There is considerable evidence that hepatitis C virus (HCV) resides in an extrahepatic reservoir. Although peripheral blood lymphocytes (PBLs) have been suspected of harboring HCV, virus production was not achieved in these cells despite many attempts. Here, we show that PBLs from HCV positive, injection drug users, harbor the virus and support viral replication. HCV replication was activated by ex vivo cell stimulation, with the use of a mixture of T and B cell activators. The presence of viral positive and negative RNA strands and HCV proteins is documented herein. Virus particles were isolated from cell supernatant and analyzed by density gradients centrifugation. Virus structural proteins and viral RNA could be readily detected in the supernatant of activated PBLs by Western blotting and real time RT-PCR, respectively. Virus particles contain de novo synthesized genomic RNA and structural proteins as shown by metabolic labeling with ³²P-orthophosphate and ³⁵S-labeled aminoacids. Finally, HCV particles, released from cells, are infectious as demonstrated by co-culturing.

Studies using this novel HCV replication system should contribute to the understanding of the virus life cycle, host-virus relationship, pathogenesis and importantly, to the discovery and validation of new anti-HCV agents.

To examine HCVs extrahepatic replication, we used PBLs from seventy-eight HCV positive, HIV-negative, injection drug users (IDUs; all obtained with written consent; table S1 detailing the available information on the participants is included in the on-line below). PBLs from the IDUs were treated with a mixture of T and B cell activators to show replication of HCV and infectivity of the de novo produced virus. The rationale behind the selection of IDUs as a source of PBLs is addressed below.

VIII. HCV(+) and (−) strand RNA and viral proteins are produced de novo in activated PBLS.

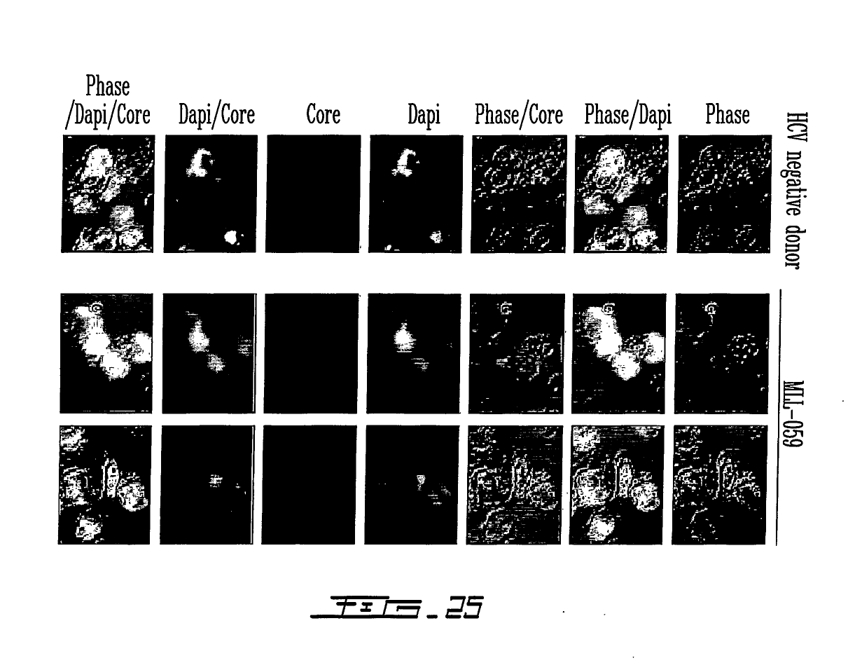

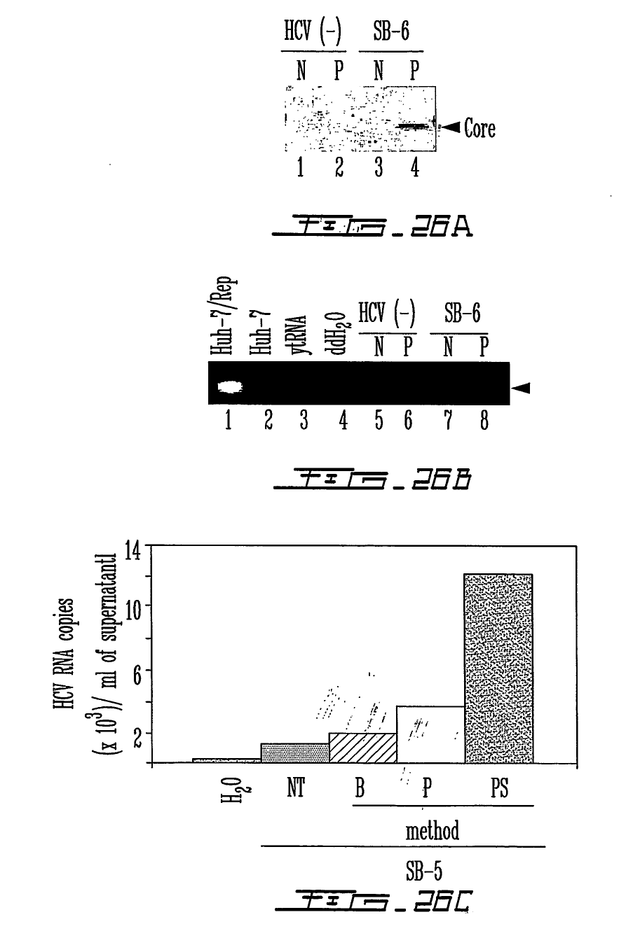

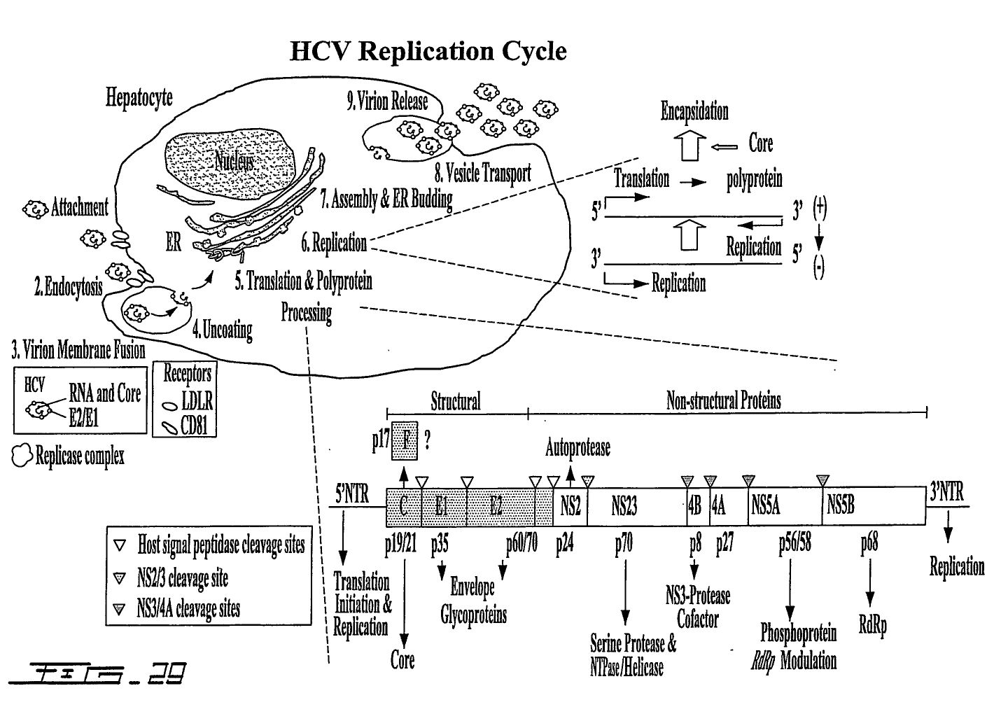

Viral RNA was detected in non-stimulated and stimulated PBLs from a HCV positive donor by nested RT-PCR (FIG. 23A). Viral RNA was not detected in HCV negative donors or in negative controls (FIG. 23A; Note that nested RT-PCR is neither strand specific nor quantitative). These results confirm early evidence showing that PBLs harbor HCV RNA (12-16). To obtain quantitative results, total RNA extracted from activated cells was subjected to a strand specific real time RT-PCR analysis to demonstrate the presence of HCV(−) RNA strand (FIG. 23B). The kinetics of HCV RNA induction was similar in activated PBLs from two carriers, MLL-038 and MLL-039 (FIGS. 23B). The amount of (−) strand RNA increases slightly, but significantly, early (1 day) upon cell activation then decreases at later times (1-3 days), but increases again afterwards (5-7 days) (FIG. 23B). Although these kinetics are not readily explained, the presence of HCV(−) RNA strand supports the notion of virus replication in PBLs. The HCV life cycle is cytoplasmic (5), therefore, to show that RNA synthesis occurs in the cytoplasm, bromo-substituted uridine (BrU) together with actinomycin D (ActD) was added to stimulated PBLs (19). Incorporated BrU was detected by immunofluorescence using antibodies to 5′-bromodeoxyuridine (19). Cytoplasmic RNA synthesis was detected in activated HCV positive PBLs from two HCV positive donors (FIGS. 23C and 28). In contrast, no incorporation of BrU was detected in ActD treated PBLs from a HCV negative donor (FIG. 23D). In the absence of ActD, strong incorporation of BrU in newly synthesized RNA was detected in the nucleus (FIGS. 23C and D). Taken together, these data clearly show that HCV RNA synthesis occurs in activated PBLs from IDUs.

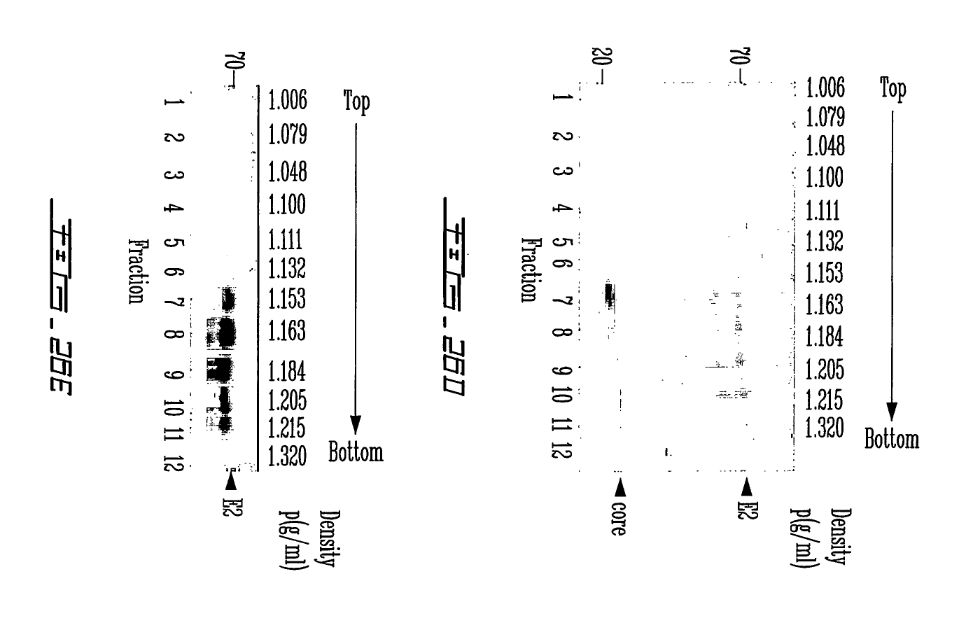

Next, we wished to document HCV-directed translation in PBLs. Upon mitogen stimulation of HCV positive PBLs, NS3 and NS5B proteins were readily detected by Western blotting using several different antibodies (FIGS. 24A-C). The quantity and kinetics of NS3 appearance was dependent on the particular procedure of stimulation (FIGS. 24D and E) and the HCV carrier (FIGS. 24F-H). This suggests that the kinetics of HCV protein production in stimulated PBLs is modulated by host factors. To show that the appearance of the proteins, which interact with the NS3 and NS5B antibodies, is dependent on HCV replication, we used siRNA against the core protein coding sequence (FIGS. 24I-K). NS3 and NS5B levels decreased drastically following electroporation of the Core-siRNA in a dose-dependent manner when compared a to a non-specific unrelated RNA (inverted 4E-T-siRNA; see Materials and Methods, below) (FIG. 24I). siRNA silencing resulted from a decrease of HCV RNA, as compared a to a non-specific RNA, as demonstrated by real-time PCR quantification (FIGS. 24J, K).

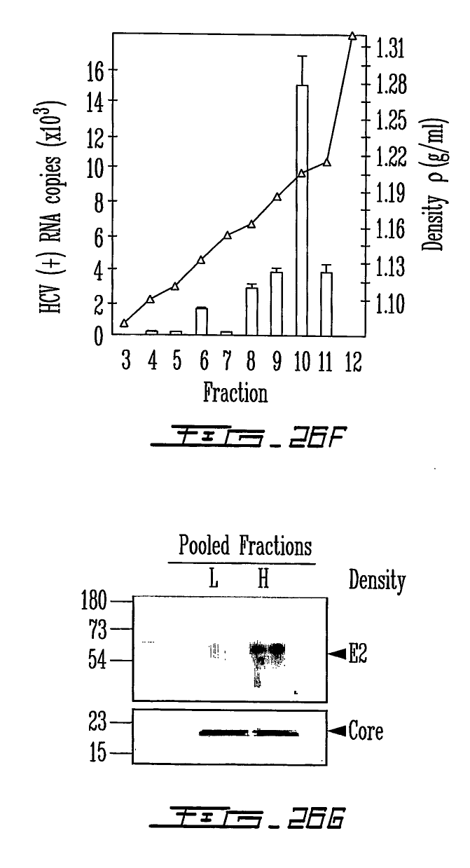

The presence of core protein in the cytoplasm of activated HCV positive PBLs was further confirmed by indirect immunofluorescence (FIG. 25). Based on surveying 10 fields, we estimate that 1 to 3% of the cells expressed high levels of HCV core protein. Taken together, the data demonstrate that translation of the HCV(+) strand RNA (FIGS. 24 and 25) and transcription of the (−) strand RNA (FIG. 23) occur in activated PBLs.

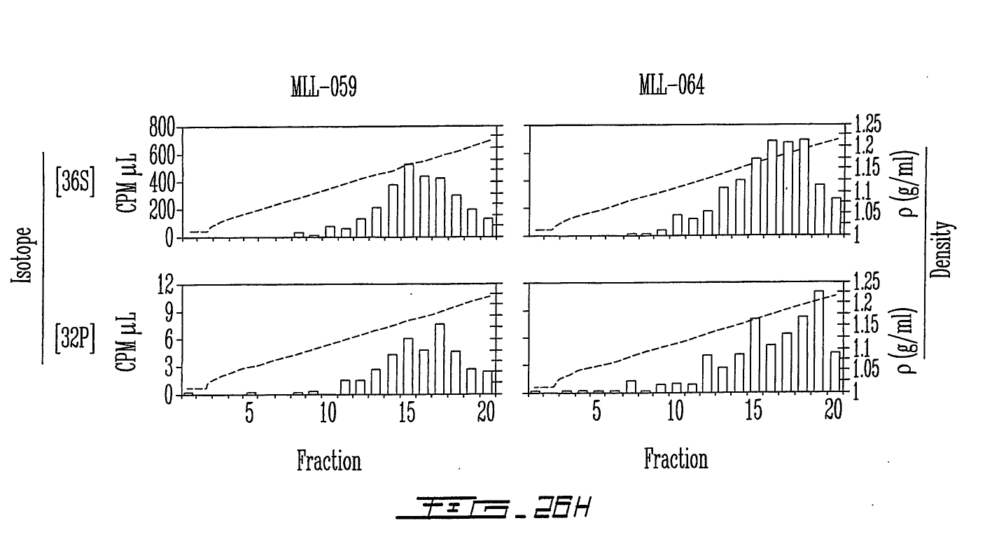

To examine whether HCV particles are produced and released into the culture medium, the supernatant from PBLs was harvested and sedimented by centrifugation through a 20% sucrose cushion. The presence of HCV particles was demonstrated by Western blotting with an anti-core monoclonal antibody, MAB225P (FIG. 26A). Similar results were obtained when other anti-core antibodies (monoclonal 515S (20) and polyclonal RR8) were used (data not shown). Viral RNA co-sedimented with the HCV core protein as demonstrated by nested RT-PCR (FIG. 26B). PBLs were stimulated by methods B, P and PS (detailed in Example 2) and genomic RNA isolated from the cell supernatant was quantified by real time RT-PCR (FIG. 26C). Consistent with the protein data shown above, the amount of viral RNA in the cell supernatant varied among the different stimulation procedures (FIG. 26C). To further support the evidence for virus production, particles were examined following metabolic labeling with 35S-methionine/cysteine (FIGS. 26D-G). Particles were sedimented through a 20% sucrose cushion, resuspended and floated on Optiprep™ density gradients (21) (FIG. 26D). The sedimentation range of the labeled particles (1.13-1.215 g/ml) was similar to that reported by others (22-28). HCV-E2 protein was present in the particles as determined by Western blotting using monoclonal anti-E2 1864 (FIG. 26E). The absolute quantity of HCV(+) strand RNA present in each faction was determined by real-time RT-PCR (FIG. 26F). The HCV genomic RNA and E2 co-sedimented through the density gradient (FIG. 26F). Interestingly, Western blotting revealed that the HCV core protein sedimented throughout the gradient (data not shown). To further examine this behaviour, fractions 1-4 and 5-11 from the gradient were pooled, and the presence of HCV E2 and core proteins was determined. The high (H) density complexes (1.111 to 1.215 g/ml) contained E2 and core protein and are likely to represent viral particles, while the low (L) density complexes (1.006 to 1.1 g/ml) contained only core (FIG. 26G). The biological significance of this observation is not immediately clear. However, it was suggested earlier that different types of particles are found in serum from chronically infected individuals (23, 29), and in the supernatant of cells expressing the full length HCV RNA (21). RNA and proteins were isolated following metabolic labeling with ³⁵S-methionine/cysteine or ³²P-orthophosphate (the latter in the presence of ActD) to determine whether the viral proteins and genomic RNA isolated from the different fractions was synthesized de novo. Supernatant was collected after labeling (FIG. 26H). Significantly, labeled RNA and proteins co-sedimented through the density gradient (FIG. 26H). Thus, the results show that virus particles containing de novo synthesized proteins and genomic RNA were released to the supernatant.

IX. HCV particles released from HCV positive PBLs are infectious.

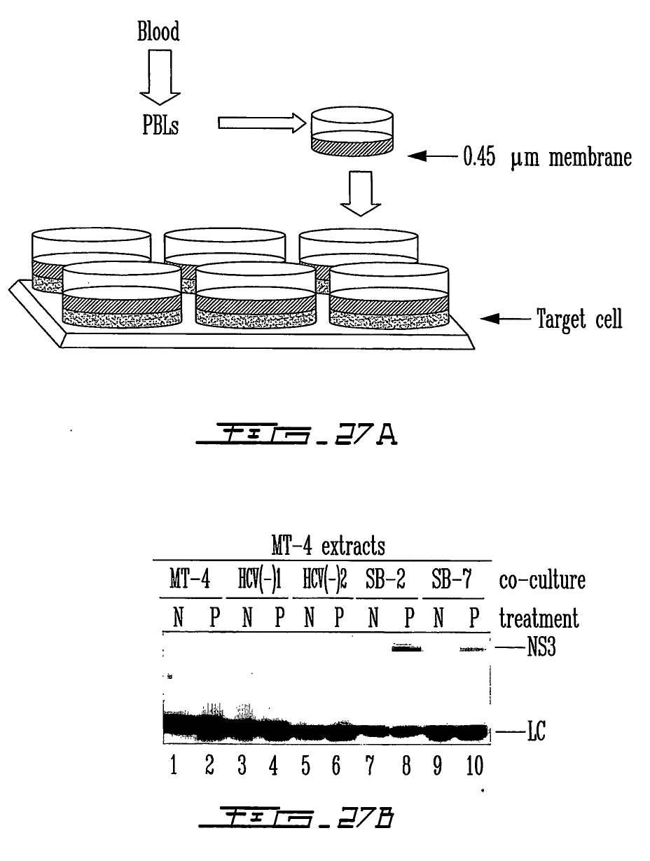

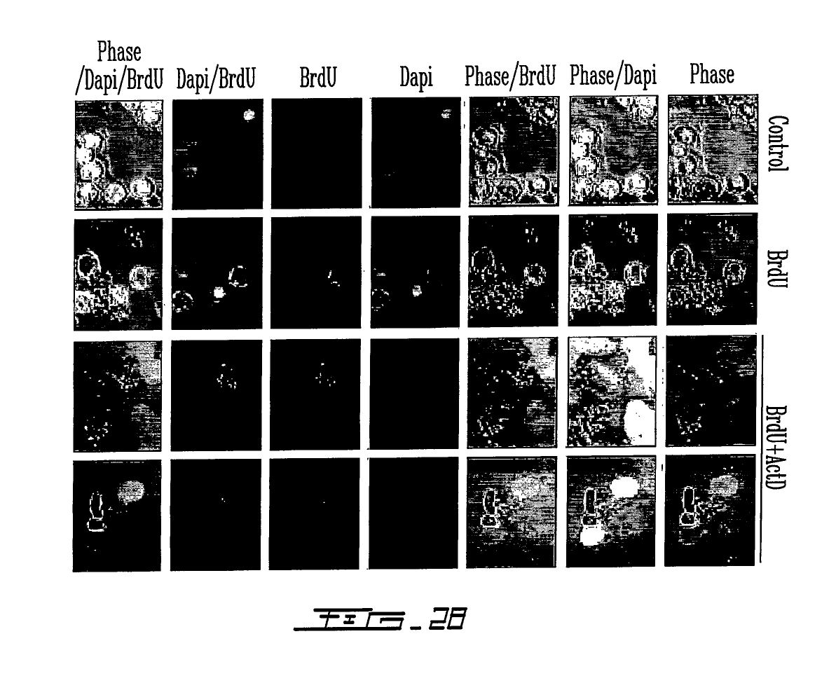

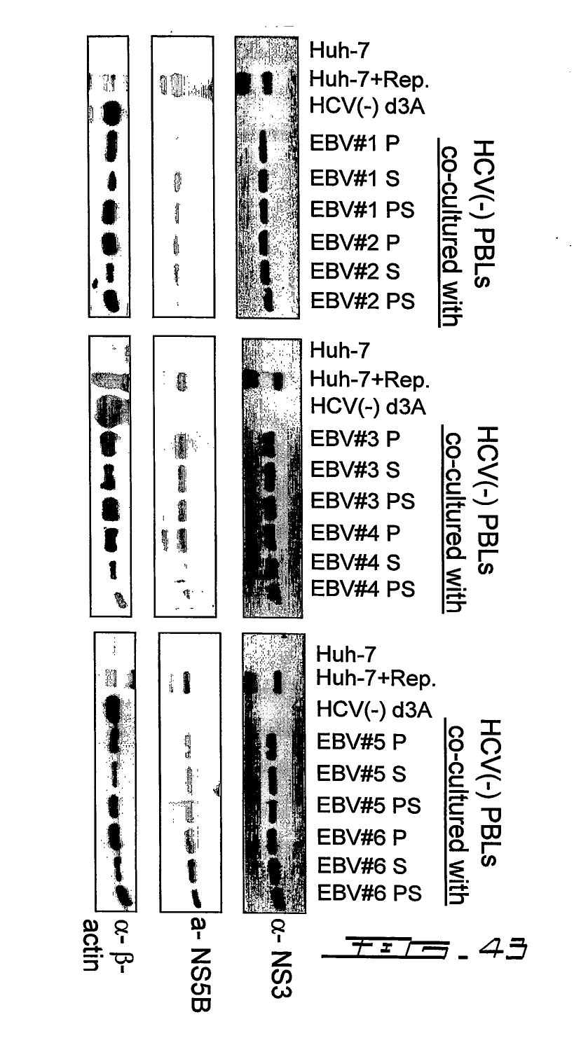

It was highly pertinent to examine whether the HCV particles released from stimulated PBLs are infectious. As it is impossible to estimate the real ratio of infectious to non-infectious virus particles produced by activated PBLs, a co-culture strategy, in which two different cell types in two chambers are separated by a 0.45 μm polyethylene terephthalate track-etched membrane, was used (FIG. 27A and FIG. 41). The HTLV-1 transformed T cell line, MT4 was chosen as the target cell of infection (30-33). Total RNA was extracted from infected cells and the quantity of HCV RNA was determined. Strikingly, viral RNA (average of 1600 copies/μg of total RNA; as determined by real-time RT-PCR, data not shown) and NS3 protein were detected in MT4, upon co-culture with activated PBLs (FIG. 27B), demonstrating that the released viral particles are infectious and that cell-to-cell contact is not required for infection. No viral proteins were detected in MT-4 cells when co-cultured with PBLs from two HCV negative donors (FIG. 27B).

In conclusion, we demonstrated that HCV replication occurs in PBLs. Without being limited to a particular theory, our success in showing replication, while earlier studies failed, can be attributed to two important factors: activation of the PBLs and the use of IDU donors. IDUs were selected because they experience a long-term altered immune response (34-36) and HCV replication in PBLs has been associated with induced immunodeficiencies (37-39). Drugs have a variety of effects on the immune system including suppressed cell-mediated immunity (34-36). This is reflected in a depressed level of T-dependent antibody production by B lymphocytes and in an alteration of T lymphocyte function. The clinical consequences of this suppression include an increase in the incidence of viral infections such as HIV and HCV(40-42). Thus, our observations support the notion that immunosuppression in combination with cell activation act as “cofactors” in HCV pathogenesis. Studies including HCV infected individuals who are not IDUs and non-IDU immuno-suppressed individuals are required to support this hypothesis.

It is most probable that HCV enters lymphocytes during the primary infection and remains latent in resting cells. Viral latency is well documented for Epstein-Barr virus (EBV), which remains dormant in quiescent host B-cells, but enters a lytic replication phase once the cell is activated (43, 44). Interestingly, EBV can also infect T cells (45, 46). Therefore, a number of intriguing parallels can be drawn between the HCV and EBV life cycles. It is conceivable that like in EBV infection, T cell immunity plays a critical role in limiting the number of HCV infected PBLs and that during a sustained immunodeficiency state, such as that manifested in IDUs, clonal proliferation of virus infected cells will be favored. Most importantly, in this report we describe a simple cell-based system that supports robust HCV replication. The implications of these findings are paramount for several reasons. First, they clearly implicate PBLs in HCV pathogenesis. Second, they provide a model that should be useful in the quest to gain understanding of the HCV life cycle, host-virus relationship, viral infectivity and in the discovery and validation of novel anti-HCV agents.

For the latter purpose we have established EBV-transformed B-cell lines from HCV-infected donors which should facilitate the discovery of anti-HCV drugs (see below). We also provide methods to establish B-cell lines which fully replicate HCV (see below).

X. Antibodies

A number of antibodies can be used in accordance with the present invention. Non limiting examples thereof include NS3 polyclonal antibody, monoclonal anti-NS5B and monoclonal anti-NS3. More specifically, monoclonal anti-NS3 antibody, 1G3D2 and polyclonal anti-NS3, K135 which were from Dr. D. Lamarre (Boehringer Ingelheim Canada Ltd). NS3 rabbit anti-serum-RB provided by Dr. R. Bartenschlager, Department of Molecular Virology, Institute of Hygiene, University of Heidelberg, Germany and monoclonal anti-NS5B, 5B-3B1 from Dr. D. Moradpour, Department of Medicine II, University of Freiburg, Germany, were also used. Monoclonal anti-E2 1864 (450-470AA), monoclonal anti-5B 10 (IFA), monoclonal anti-Core 515S (2040AA), and Core rabbit anti-serum RR8 were developed in The Tokyo Metropolitan Institute of Medical Science. Monoclonal anti-Core (Cat.No.: MAB255P; Lot:hcv-core-2-4) was purchased from Maine Biotechnology services, Inc. Monoclonal anti-human F-Actin (ab205) was purchased from Abcam Limited. Monoclonal anti-human β-Actin (clone AC-15) was purchased from Sigma-Aldrich CO. Anti-Bromodeoxyuridine monoclonal antibody-Alexa fluor 488 conjugated, and goat anti-rabbit Alexa fluor 594 conjugated were purchased from Molecular Probes, Inc. Of course, other antibodies (or combination of antibodies), whether polyclonal or monoclonal can also be used.

XI. Blood Donors and Lymphocyte Purification

Participants were recruited through the drug addiction unit of the Saint-Luc Hospital of the Centre Hospitalier de l'Université de Montral (CHUM) and the Saint-Luc Cohort study. Characteristics of the donors can be found at Table S1. Donors provided a written informed consent approved by the CHUM Review Board before having their blood drawn. Individuals from both sexes (87% males) were enrolled in this study between 2001 and 2003. Their mean age was 42.1 years (sd±8.8) and the average time since their first injection was 16.5 years (sd±9.6). 80% of the donors reported injecting drugs during the 6 month period before blood was withdrawn for this study. Cocaine and opiates were the most frequently used drugs, with 77% and 34.6% use, respectively. All HCV positive donors tested positive in a serological screen for HCV antibodies performed in the laboratory of microbiology at Saint-Luc Hospital of the CHUM using two Enzyme Linked Immunosorbent Assays (ELISA, AxSym and Cobas). Presence of HCV was confirmed by HCV-RNA detection when ELISA data were discordant. All participants recruited for this study were HIV-1 and HIV-2 negative. Serological screening for HIV antibodies was performed in the microbiology laboratory at Saint-Luc Hospital, CHUM, with an enzyme-linked immunosorbent assay (ELISA). Similar procedures were used to verify the HCV negative donors. HCV negative donors (six) were recruited from the different participating laboratories as well as from the support staff responsible for the St. Luc Cohort. Peripheral blood (20 ml) was collected from HCV positive IDU or HCV negative donors into EDTA-containing Vacutainer tubes (Becton Dickinson). Polymorphonuclear leukocytes and red blood cells were separated by centrifugation over a density gradient (Lymphocyte separation medium, cellgro®). Monocytes were then removed by plastic adherence under serum free conditions as described in The Current protocols of Immunology textbook. When required, cells were frozen in 10% DMSO containing FCS and stored at −80° C. prior to monocyte separation. Total PBLs were cultured in 24-well plates at 1×10⁶ cells per ml in RPMI 1640 supplemented with 10% heat-inactivated FCS and antibiotics.

XII. PBLs Stimulation

Mitogens were added to the media (RPMI 1640, 10% FBS, and antibiotics) upon starting the culture and maintained throughout the experiment. The protocols used for PBCLs stimulation were as follows: Method A, PBLs were grown in the presence of irradiated L4.5 cells (murine fibroblasts expressing the CD40 ligand, CD154) as described (49). Method B, 1 μg/ml of anti-CD3 and 200 U/ml of IL-2 (Sigma-Aldrich CO) were added. Method P, 3 μg/ml phytohemagglutinin (PHA, Sigma-Aldrich CO), and 200 U/mi IL-2 were used. Method PS, 1:10⁴ vol/vol of Staphylococcus aureus Cowan fixed cells (SAC, Calbiochem) in combination with phytohemagglutinin and 200 U/ml IL-2 were added to the media. Method S, 1:10⁴ vol/vol of SAC and 200 U/ml of IL-4 (Sigma-Aldrich CO) were used.

Cell activation was verified by flow cytometry. Cells were rinsed twice with 1 ml cold phosphate buffered saline (PBS: 137 mM NaCl, 2.7 mM KCl, 4.3 mM Na₂HPO₄, 1.4 mM KH₂PO₄, pH 7.4) and fixed in 80% ethanol/PBS for 30 min at 4° C. PBS (2 volumes) was added and cells were pelleted by centrifugation. Cells were rinsed twice with 2 ml PBS and then resuspended in 0.5 mL PBS containing 0.2 μg/ml RNase A and incubated for 40 min at 37° C. Propidium iodide was added to a final concentration of 1.2 μg/ml and samples were analyzed by flow cytometry using a single laser FACS instrument (Becton-Dickinson) combined with the CellQuest™ software.

XIII. RNA Purification

Total RNA was extracted from cells using Trizol™ (Invitrogen) according to the manufacturer's protocol. Yeast tRNA (1 mg/ml) was added as a carrier. RNA was resuspended in nuclease-free water (Sigma-Aldrich CO). Total RNA was quantified by Phosphoimagerm (STORM system, Molecular Dynamics) using the RiboGreen™ RNA Quantification Kit (Molecular Probes, Inc).

XIV. Nested RT-PCR

HCV-RNA was detected in cells by a reverse transcription-polymerase-chain reaction (one step RT-PCR reaction, 45 cycles, Qiagen) against the highly conserved 5′ untranslated region (sense primer from nucleotide 13 to 38 and the anti-sense primer from nucleotide 383 to 359) of the HCV genome (strain H77 pCV-H77C, EMBL:AF011751, MEDLINE: 97385173) followed by a second round of amplification, nested PCR (45 cycles, sense primer from nucleotide 59 to 82 and the anti-sense primer from nucleotide 307 to 285, strain H77 pCV-H77C) using Taq DNA polymerase (MBI Fermentas). β-Actin was amplified (30 cycles) using the sense primer 5′-GTGGGGCGCCCCAGGCACCA-3′ and antisense primer 5′-GTCCTTAATGTCACGCACGATTTC-3′.

XV. Real Time RT-PCR

Two methods were used to detect and quantify HCV RNA.

Method I: Reverse transcription was carried out at 50° C. for 20 minutes in a one-tube two-step RT-PCR reaction with Thermoscript™ reverse transcriptase (Invitrogen), 10 μM of HCV-tagged strand-specific RT primer and 100 μM of anti-sense GAPDH primer (Table S2). The reverse transcriptase was inactivated by heating for 5 minutes at 95° C. and PCR (22 cycles) with Platinum Taq DNA polymerase was performed in a Trio-thermocycler™ (Biometra): at 94° C. for 45 s, 60° C. for 60 s, 68° C. for 2 min. The first round PCR products were then amplified for 40 cycles in the Roche LightCycle™ instrument: denaturation at 95° C. for 60 s, and amplification and quantification at 95° C. for 15 s, 60° C. for 10 s with a single fluorescence measurement, 72° C. for 15 s. Real-time quantification of RNA copy numbers for HCV and the human GAPDH gene was based on a set of eight log₁₀ external standards covering 10⁸ to 10¹ plasmid copies of a pCRII vector containing the 5′ HCV leader (genotype 1a) and the GAPDH normalization PCR amplicons which were run in parallel with the test samples. RNA extracted from PBLs of a HCV negative donor was used as control. As a reaction control for the strand-specific signal, the RT step of the RT-PCR was carried out without a HCV-tagged primer. The presence of HCV non-structural proteins in the cell samples used for RNA preparation was confirmed by Western blotting (data not shown).

Method II: Real-time RT-PCR was performed on the ABI Prism 7700 Sequence Detection System using the TaqMan EZ RT-PCR Kit (Applied Biosystems). RNA sample (5 μl), combined with 45 μl of Reagent Mix, was used for the Real-Time RT-PCR reaction. In vitro transcribed replicon RNA was used as a standard to determine HCV copy numbers (1 μg of replicon RNA equals 2.15×10¹¹ HCV copies). The RNA copy number was normalized (RiboGreen™ RNA quantification, Molecular Probes Inc.) and expressed as genome equivalents per ml of total supernatant.

XVI. Bromo-Uridine Labeling

Bromo-uridine (BrU, 5-Bromouridine 5′-Triphosphate, Sigma-Aldrich CO) was incorporated into PBLs using a modified version of the procedure of Haukenes et al. (50). BrU (10 mM) was incubated with an equal volume of Lipofectamine™ 2000 transfection reagent (Invitrogen) for 30 min at room temperature and added to 250 μl of cells resuspended in optimem™ medium (Invitrogen) in a 1:1 (vol/vol) ratio. The BrU/Lipofectamine™ 2000 mixture was added to cells 6 h after activation. Cells were incubated for 5 h, washed and resuspended in mitogen (method P) containing culture medium. Cells were collected after a 12 h incubation period at 37° C. in a 5% CO₂ environment. When actinomycin D (ActD, Sigma-Aldrich CO) was used, cells were incubated with the drug (5 μg/ml) starting 30 min prior to the addition of BrU. ActD was maintained throughout the experiment.

XVII. Immunofluorescence

Immunofluorescence was performed on 5×10 ⁴ cells. Following cytospin for 7 min at 1100 rpm in a Cytosin 2 (Shandon), cells were dried for 30 min at room temperature and fixed for 30 min at −20° C. in a mixture of acetone and methanol (1:1 vol/vol). Cells were blocked for 30 min at room temperature in 10 mM Tris-HCl pH 8.0 containing 1% BSA. Slides were washed 3 times with PBS and incubated at room temperature for 2 h with the polyclonal anti-core RR8 antibody (1/50) or overnight at 4° C. with the anti-bromo-deoxyuridine Alexa Fluor 488 conjugate antibody (2 μg/ml) in a humidified box. Slides were washed 3 times with PBS. For Core detection, slides were incubated 1 h at room temperature with an Alexa-594 conjugated antibody (dilution 1/250). DAPI staining was performed for 7 min at room temperature (1 μg/ml final concentration). Mounted slides (Permount mounting medium, Fisher Scientific) were stored overnight at 40° C. prior to analysis. Conventional epifluorescence micrographs were obtained on a Zeiss Cell Observer™ system equipped with an Axiovert™ 200 M microscope using the 100× oil lens. Images were digitally deconvoluted with the AxioVision 3.1™ software using the Nearest Neighbor™ deconvolution method that uses the Agard's formula.

XVIII. Western Blots

Proteins extracts were prepared by sonification in RIPA buffer (150 mM NaCl, 1% NP-40, 0.5% DOC, 0.1% SDS, 50 mM Tris-HCl pH 7.5) and quantified (BSA assay, BioRad). Proteins (10 Ag of extracts from PBLs or 5 μg of extract from Huh7 cells stably expressing the HCV replicon (47)) were resolved on SDS-10% polyacrylamide gels (PAGE) and transferred to 0.2 pm Protran™ nitrocellulose membrane (Schleider and Schuell) for 1 h at 100V. The membrane was blocked with PBS containing 0.5% Tween-20 (PBS-T) and 5% nonfat dry milk. Blots were then incubated with the primary antibody for 2 h at room temperature, washed 3 times with PBS-T and incubated for 1 h with a horse radish peroxidase (HRP) conjugated secondary antibody. Blots were visualized using an enhanced luminol reagent (ECL; PerkinElmer Life Sciences Inc).

XIX. Radio Labeling and Gradient Purification of Virus Particles

A total of 1×10⁶ activated PBLs were first preincubated in methionine- or phosphate-free RPMI for 30 min, and then incubated for 12 h in the same media supplemented with [35S] protein labeling mix (1175 Ci/mmol) or carrier-free inorganic ³²P (500 μCi/ml, H₃PO₄, ICN Biomedicals, INC), the latter in presence of ActD (5 μg/ml). Supernatant was collected, cells and cellular debris was removed by low-speed centrifugation at 1600× g for 15 min at 40° C., followed by filtration with 0.45 μm pore size filter (Fisherbrand, Fisher scientific). Particles were partially purified by ultracentrifugation through a 20% sucrose cushion for a minimum of 6 h at 4° C. (in Beckman L8-55 ultracentrifuge) at 35,000 rpm in a SW-41 rotor. Sediments were resuspended in serum free RPMI and lodixanol (Optiprep™, Invitrogen) was added to a final concentration of 40% w/v (p=1.216). The sample was laid over a 60% wt/vol Optiprep™ solution (p=1.320 g/ml) and then overlaid with a linear iodixanol gradient (p=1.038 to 1.205 g/ml) prepared in RPMI and spun for 20 h at 4° C. in Beckman L8-55 ultracentrifuge at 30,000 rpm using a SW-41 rotor. Fractions were collected from the top of the tube and RNA was prepared as described above. Half of the final RNA volume was mixed with liquid scintillation cocktail (EcoLite™, ICN Biomedicals) and ³²P radioactivity was counted in a Beckman LS 6500 scintillation counter. Proteins were extracted by directly adding 10× RIPA buffer to a final concentration of 1× RIPA. 1/100th of the protein extract was mixed with liquid scintillation cocktail and 35S radioactivity was determined using a Beckman LS 6500 scintillation counter. 1/10 of the protein extract was directly mixed with concentrated Laemmli sample buffer, resolved on a SDS 15%-PAGE, and transferred to 0.2 μm Protran nitrocellulose membrane over night at 30V. The membrane was dried and exposed against Kodak Biomax™ MR film. The remaining protein extract was concentrated by TCA precipitation (15% final). Proteins were washed twice with ether, dried and dissolved in a solution containing 3 M urea, 26 mM EDTA (pH 8), and 0.5 μg/ml of RNase A. Samples were mixed with concentrated Laemmli sample buffer, resolved on a SDS 10% PAGE and transferred to 0.2 μm Protran nitrocellulose membrane for 1 h at 100V. Proteins were detected by Western blotting as described above.

XX. siRNA

The target sequence for the siRNA was chosen using the Ambion™ web-based criteria. The selected RNA oligonucleotides, Core (from nucleotide 371 to nucleotide 391, strain H77 pCV-H77C, EMBL:AF011751, MEDLINE: 97385173) and the unrelated non-specific RNA (inverted sequence for 4E-T from nucleotide 986 to nucleotide 1008; DDBJ/EMBL/GenBank™ database, accession No. AF240775), were synthesized by Dharmacon Research (Lafayette, Colo.) and handled according to the manufacturer's instructions. Varying amounts (3 μ; or 5 μl of a 20 μM solution) of RNA duplexes were electroporated using a Gene pulser® II electroporator (BioRad), into 1×10⁶ PBLs in 0.5 ml of serum free RPMI. Cells were treated with a pulse of 975 μF and 300 V. Then 0.5 ml of RPMI containing 20% FCS was added and the cells were seeded in a 24-well cell culture dish. Protein and RNA extracts were harvested 48 h after electroporation. Immunoblots were performed as described above using an NS3 rabbit antiserum-RB and monoclonal anti-NS5B, 5B-3B1. HCV RNA levels were quantified by real-time RT-PCR using method I.

TABLE S1
Characteristics of the IDU donors, enrolled between
March 2001 and April 2003:

| | | | | | | Opioids excl | |
|---|---|---|---|---|---|---|---|
| | | | IDU | Under | IDU | methdone | Cocaine |
| | Age | | duration | Methadone | (past 6 | (past 6 | (past 6 |
| Participant | (years) | Sex | (years) | treatment | months) | months) | months) |
| SB-1 | 41 | male | 22 | yes | no | no | no |
| SB-2 | 42 | female | 20 | yes | yes | yes | yes |
| SB-4 | 35 | male | 11 | yes | yes | yes | yes |
| SB-5 | 21 | female | 3 | yes | yes | yes | no |
| SB-6 | 32 | male | 1 | yes | yes | yes | yes |
| SB-7 | 45 | male | 18 | yes | no | no | no |
| MLL 001 | 48 | male | 31 | no | yes | yes | yes |
| MLL 002 | 39 | male | 3 | no | yes | no | yes |
| MLL 003 | 38 | male | 10 | no | yes | yes | yes |
| MLL 004 | 47 | male | 32 | no | yes | yes | yes |
| MLL 005 | 38 | male | 21 | no | yes | no | no |
| MLL 006 | 49 | male | 37 | yes | yes | yes | no |
| MLL 007 | 61 | male | 36 | no | yes | no | no |
| MLL 008 | 39 | male | 13 | no | no | no | yes |
| MLL 009 | 23 | male | 5 | no | yes | no | no |
| MLL 010 | 40 | male | 21 | no | no | no | yes |
| MLL 011 | 45 | male | 6 | no | yes | no | yes |
| MLL 012 | 48 | male | 14 | no | yes | yes | yes |
| MLL 013 | 49 | male | 24 | no | no | yes | no |
| MLL 014 | 41 | male | 18 | no | yes | no | yes |
| MLL 015 | 38 | male | 6 | no | yes | yes | yes |
| MLL 016 | 34 | male | 11 | no | no | no | no |
| MLL 018 | 42 | male | 13 | no | yes | no | yes |
| MLL 019 | 51 | male | 10 | no | yes | no | yes |
| MLL 020 | 38 | male | 13 | no | yes | no | yes |
| MLL 021 | 35 | female | 5 | no | no | no | no |
| MLL 022 | 43 | male | 29 | no | yes | no | yes |
| MLL 023 | 52 | male | 20 | no | yes | no | yes |
| MLL 024 | 37 | male | 13 | no | yes | no | yes |
| MLL 025 | 36 | male | 18 | yes | yes | yes | yes |
| MLL 026 | 29 | female | 13 | yes | yes | yes | yes |
| MLL 027 | 52 | male | 11 | no | yes | yes | yes |
| MLL 028 | 45 | male | 6 | no | yes | yes | yes |
| MLL 029 | 42 | male | 6 | no | yes | yes | yes |
| MLL 030 | 43 | male | 10 | no | yes | no | yes |
| MLL 031 | 36 | male | 19 | yes | yes | no | yes |
| MLL 032 | 22 | male | 11 | yes | yes | yes | no |
| MLL 033 | 24 | male | 7 | yes | yes | yes | yes |
| MLL 034 | 52 | male | 26 | no | yes | no | yes |
| MLL 035 | 61 | male | 36 | no | yes | no | no |
| MLL 036 | 49 | male | 31 | no | yes | no | yes |
| MLL 037 | 57 | male | 36 | no | no | no | no |
| MLL 038 | 27 | male | 11 | no | yes | yes | yes |
| MLL 039 | 42 | female | 17 | yes | yes | yes | no |
| MLL 040 | 53 | male | 40 | no | yes | yes | yes |
| MLL 041 | 34 | male | 11 | no | no | no | yes |
| MLL 042 | 47 | male | 7 | no | yes | no | yes |
| MLL 043 | 42 | female | 23 | no | no | no | no |
| MLL 044 | 30 | male | 11 | no | no | no | yes |
| MLL 045 | 41 | male | 22 | no | yes | no | yes |
| MLL 046 | 43 | male | 21 | no | yes | yes | yes |
| MLL 047 | 41 | male | 18 | no | yes | no | yes |
| MLL 048 | 47 | male | 22 | no | yes | no | yes |
| MLL 049 | 52 | male | 11 | no | no | no | yes |
| MLL 050 | 33 | male | 10 | no | yes | no | yes |
| MLL 051 | 45 | male | 30 | yes | yes | no | yes |
| MLL 052 | 33 | male | 8 | no | yes | no | yes |
| MLL 053 | 43 | female | 12 | no | yes | no | yes |
| MLL 054 | 46 | male | 22 | no | yes | no | yes |
| MLL 055 | 36 | female | 21 | yes | yes | no | yes |
| MLL 056 | 40 | male | 14 | no | no | no | yes |
| MLL 057 | 37 | male | 9 | no | yes | yes | yes |
| MLL 058 | 45 | male | 30 | yes | yes | no | yes |
| MLL 059 | 50 | male | 30 | no | yes | yes | yes |
| MLL 060 | 35 | male | 12 | yes | yes | yes | no |
| MLL 061 | 46 | male | 7 | no | no | no | yes |
| MLL 062 | 48 | male | 11 | yes | yes | yes | yes |
| MLL 063 | 66 | female | 35 | no | yes | no | yes |
| MLL 064 | 38 | male | 3 | no | yes | no | yes |
| MLL 065 | 33 | male | 10 | no | yes | no | yes |
| MLL 066 | 48 | male | 11 | yes | no | no | no |
| MLL 067 | 46 | male | 11 | no | yes | no | yes |
| MLL 068 | 42 | male | 6 | no | yes | no | yes |
| MLL 069 | 42 | male | 23 | no | yes | yes | yes |
| MLL 070 | 44 | male | 11 | no | yes | no | yes |
| MLL 071 | 47 | female | 22 | no | yes | no | yes |
| MLL 072 | 61 | male | 16 | no | yes | no | yes |
| MLL 073 | 37 | male | 9 | yes | no | no | no |

TABLE S2
Probes and primers used in Real Time RT-PCR method I.

| Name | Orientation | Used in: | Target | Nucleotide position in target |
|---|---|---|---|---|
| H250 | sense 5′ external | | HCV | 64-84 |
| HC110 | antisense 3′ external | RT and | HCV | 456-475 |
| | | 1^{st} round PCR | | |
| G.24 | sense 5′ external | | GAPDH | 15-32 |
| G.589 | antisense 3′ external | | GAPDH | 581-597 |
| H190 | sense 5′ internal | | HCV | 142-161 |
| C40 | antisense 3′ internal | real-time PCR | HCV | 385-405 |
| G.174 | sense 5′ internal | | GAPDH | 166-182 |
| G.511 | antisense 3′ internal | | GAPDH | 502-520 |
| 297.P1 | sense FL1 probe | | HCV | 274-297 |
| 300.P2 | sense FL2 probe | Hybridization | HCV | 300-324 |
| | | Probes | | |
| G.P1 | antisense FL1 probe | | GAPDH | 187-212 |
| G.P2 | antisense FL2 probe | | GAPDH | 214-238 |

XXI. Established EBV-Transformed Cell Line Enabling Robust Hepatitis C Virus Replication in PBLS from HCV Infected Donors

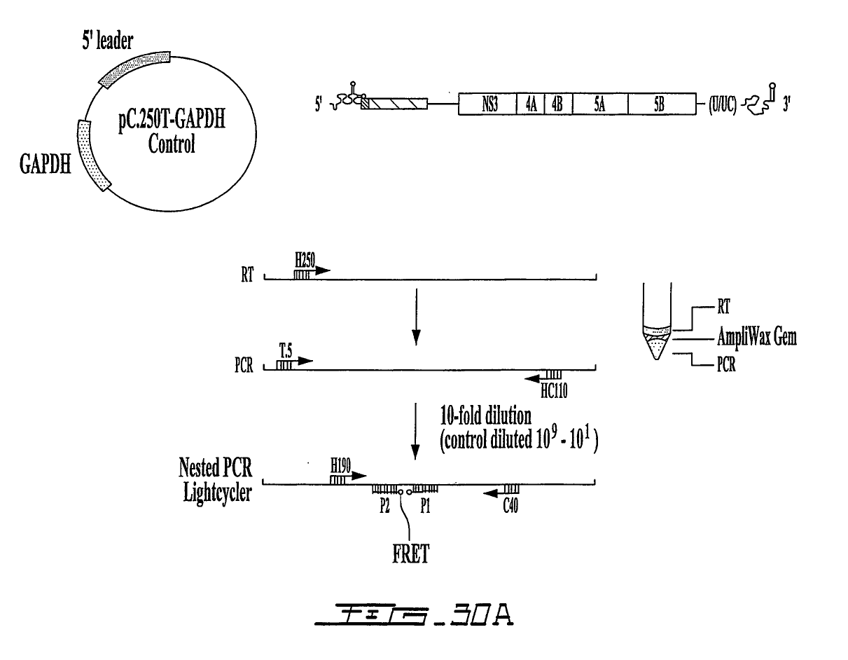

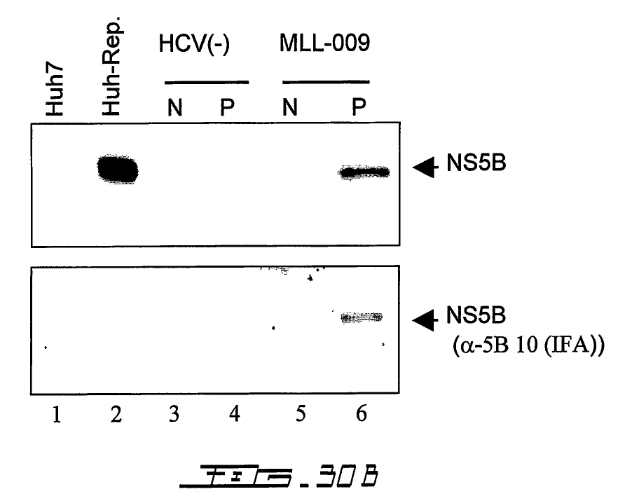

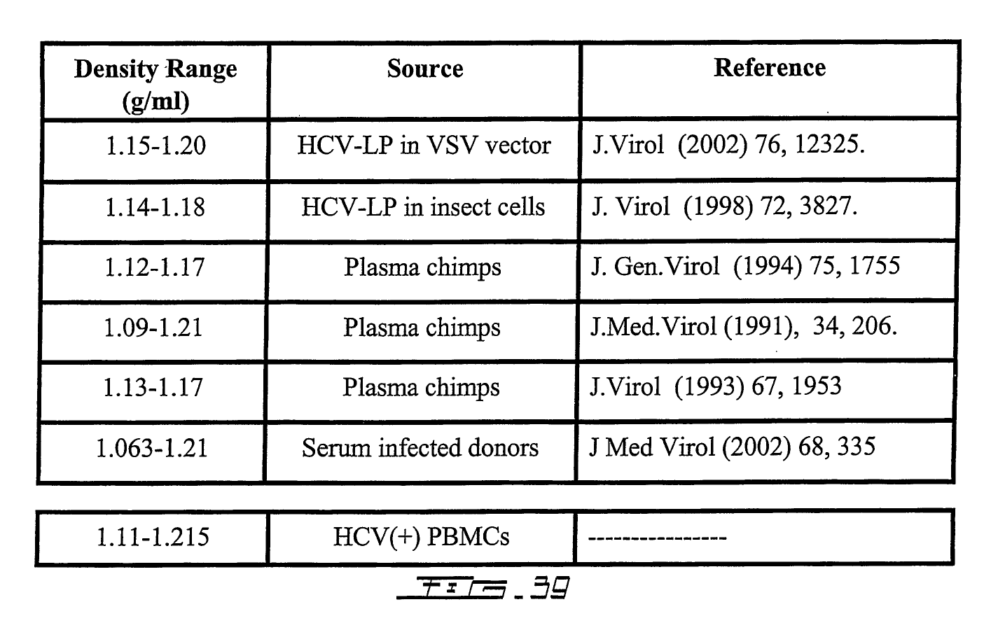

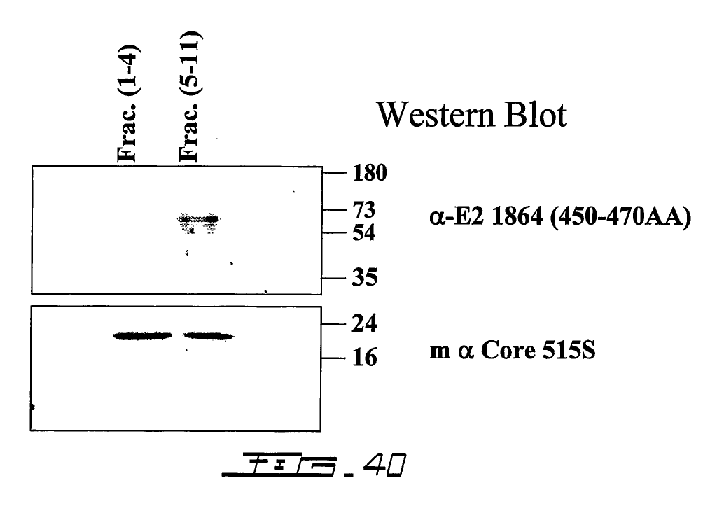

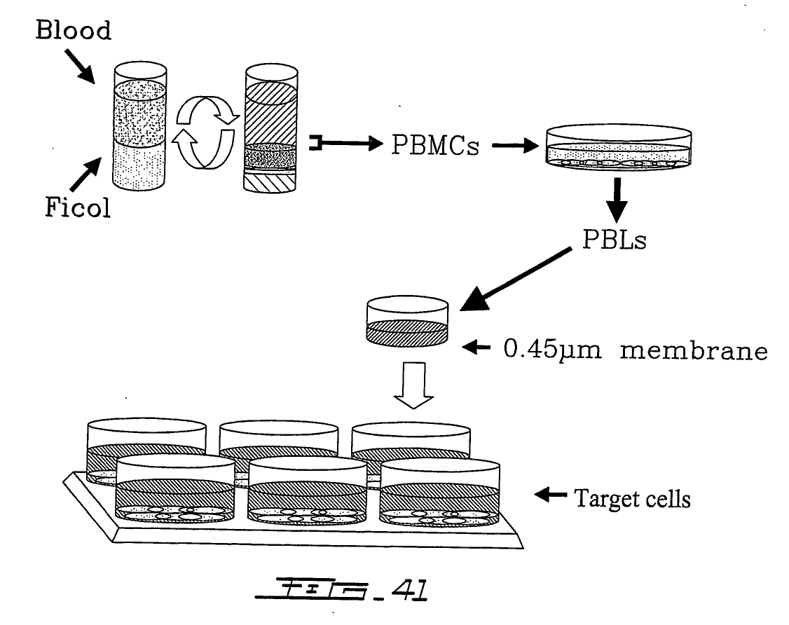

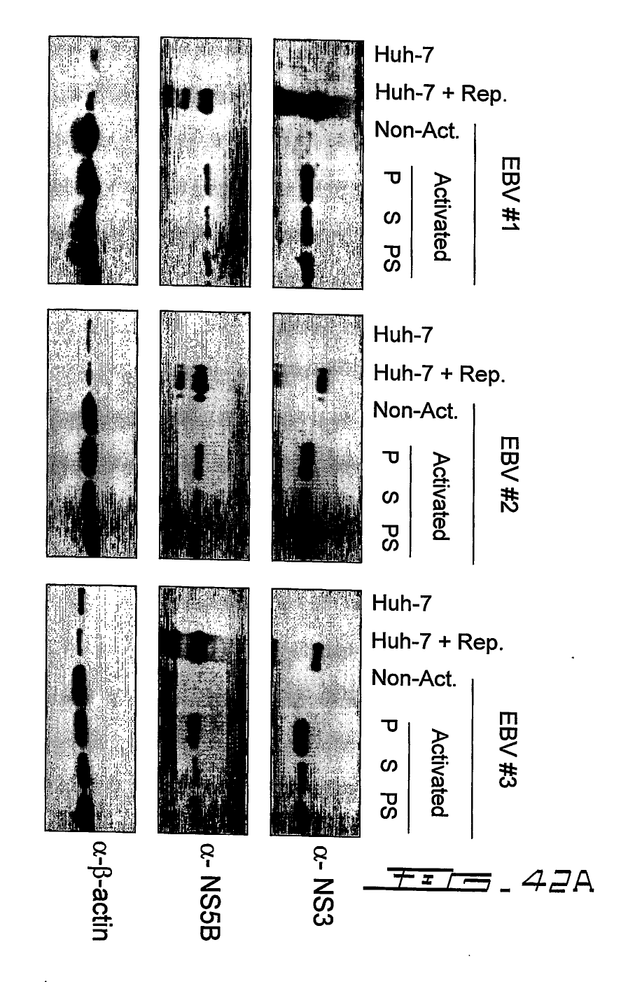

It is shown herein that HCV can naturally infect blood cell and can replicate therein (FIGS. 29-41). In order to assess whether the produced HCV was infectious the protocol of FIG. 41 was followed. We show that HCV replicating in naturally infected PBLs was indeed infectious. We further went on to generate an HCV expressing cell line. In an embodiment, we developed an EBV-cell line that is able to replicate HCV.

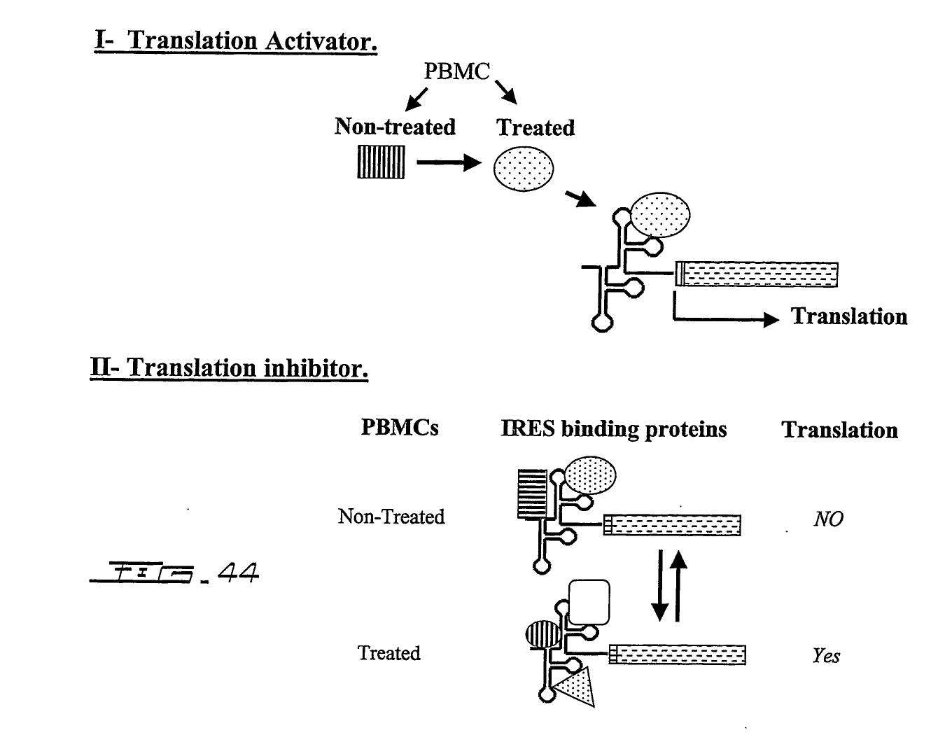

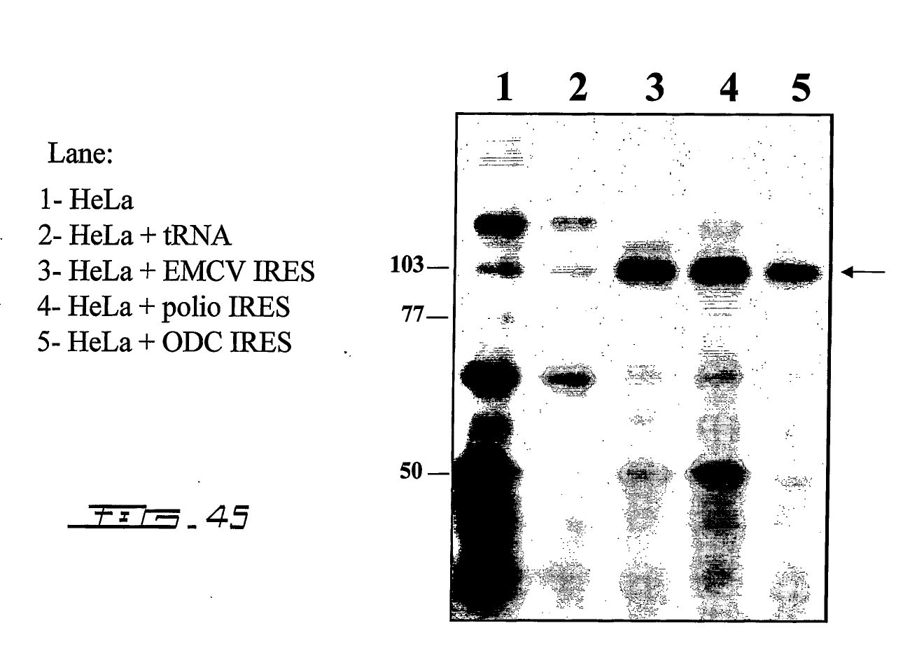

B-cells from infected donors were identified as the cells that harbored HCV virus. These cells were immortalized by EBV infection. Interestingly, when grown under normal conditions, the EBV-immortalized B cells from infected donors, do not produce detectable amounts of HCV proteins. However, when stimulated (independent from the stimulation procedure P, S or PS) virus proteins (NS3 and NS5) become detectable (FIG. 42).

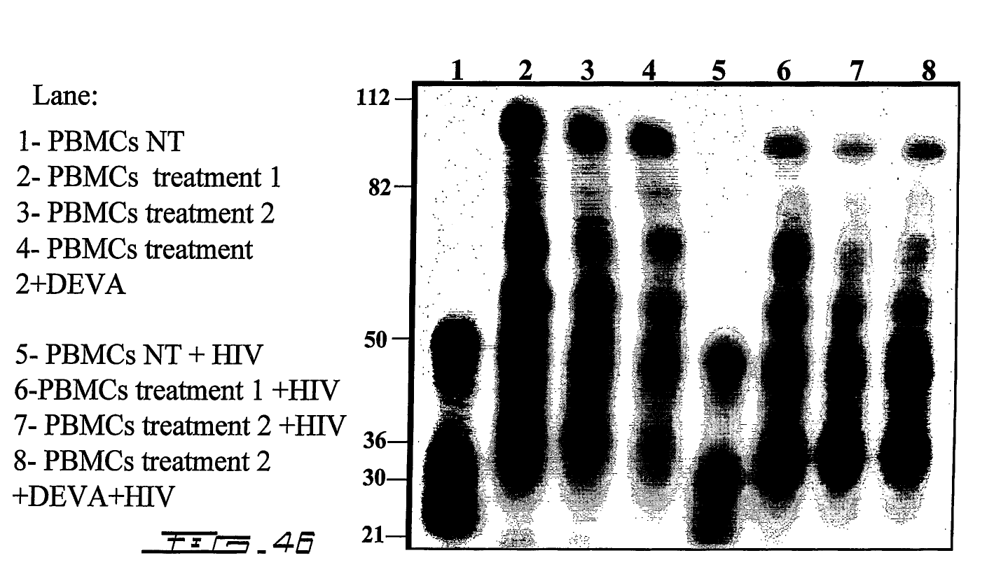

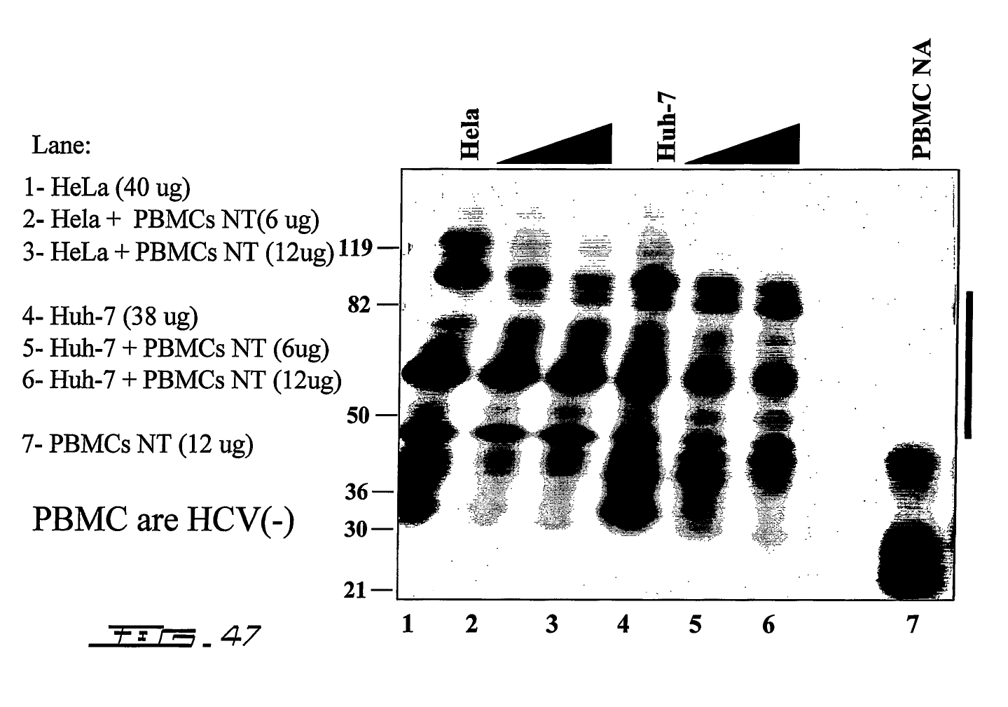

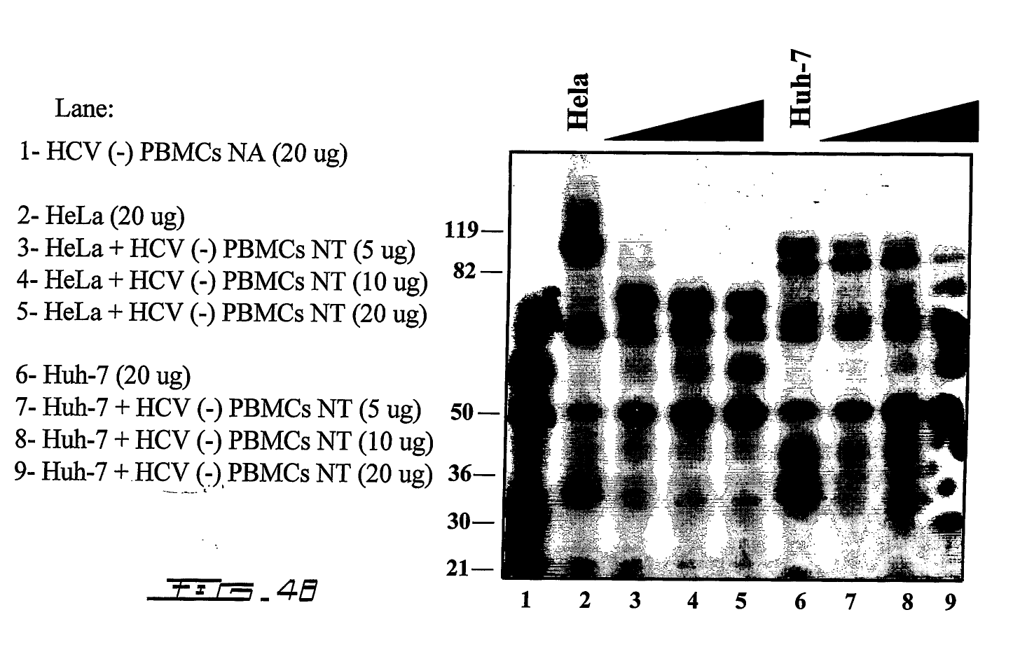

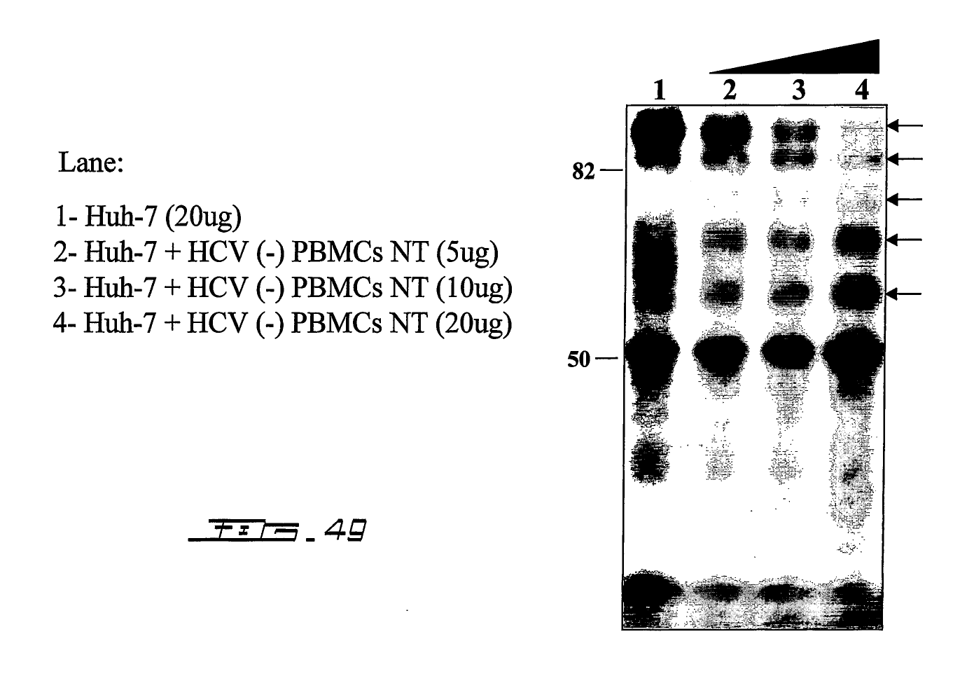

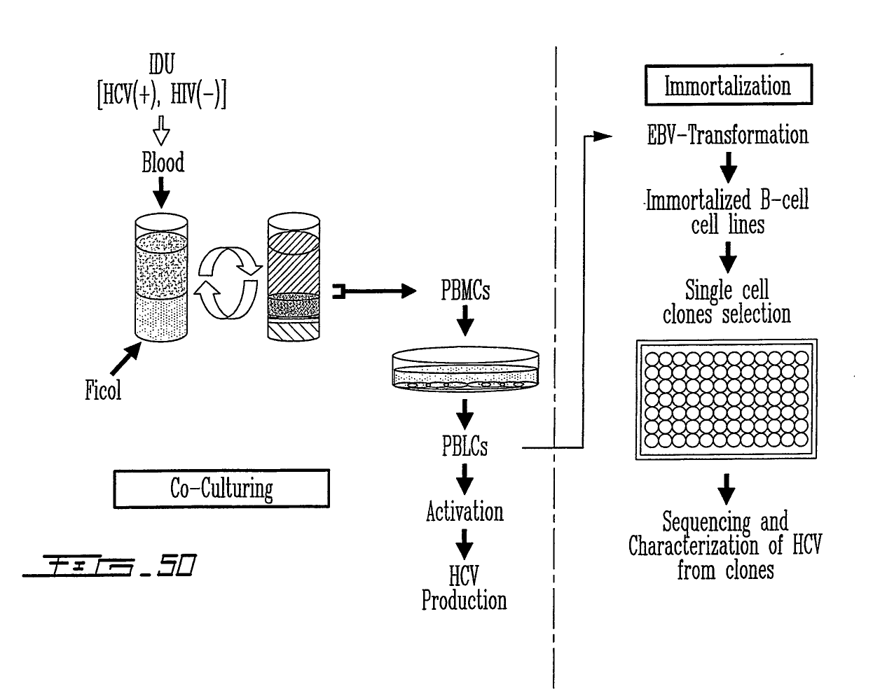

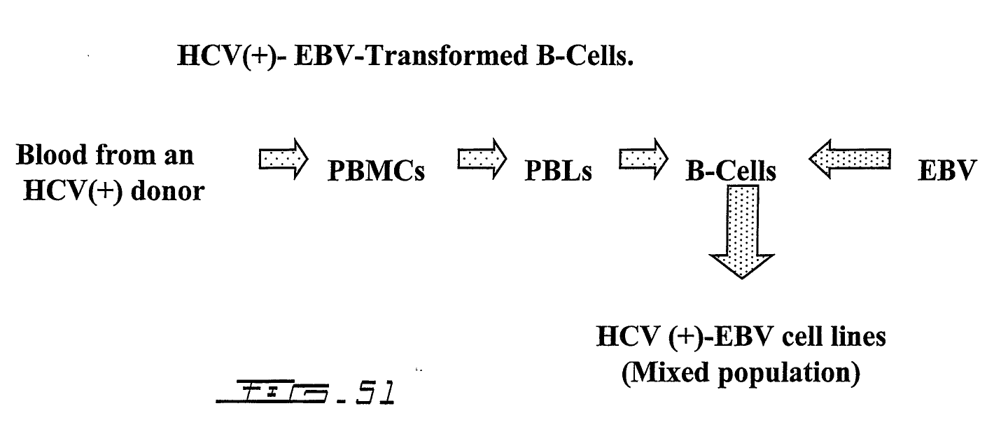

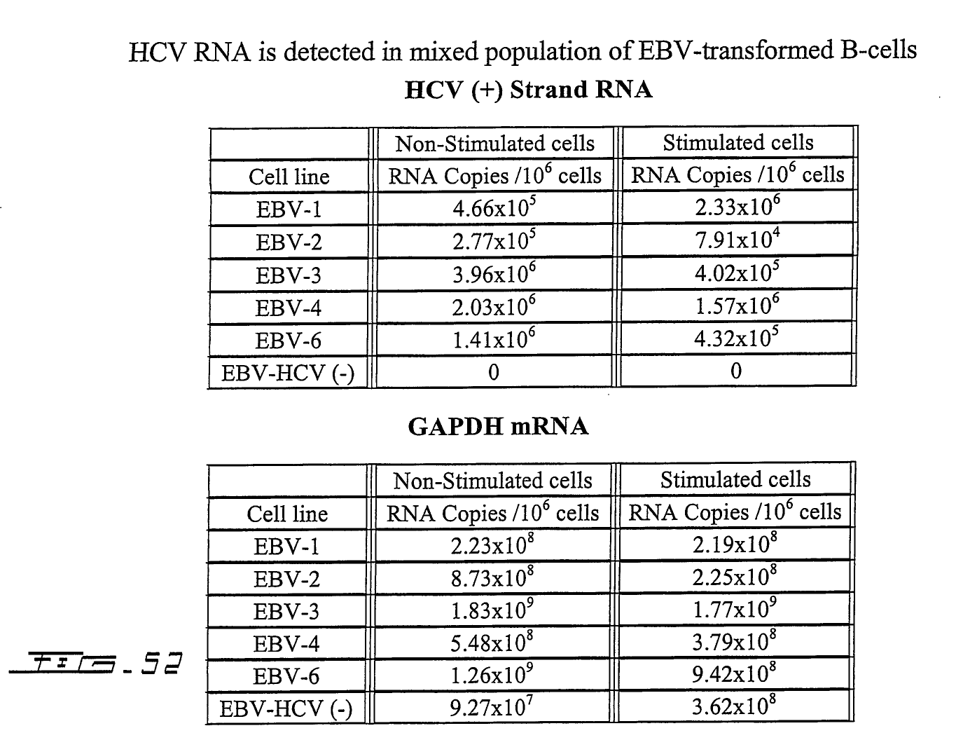

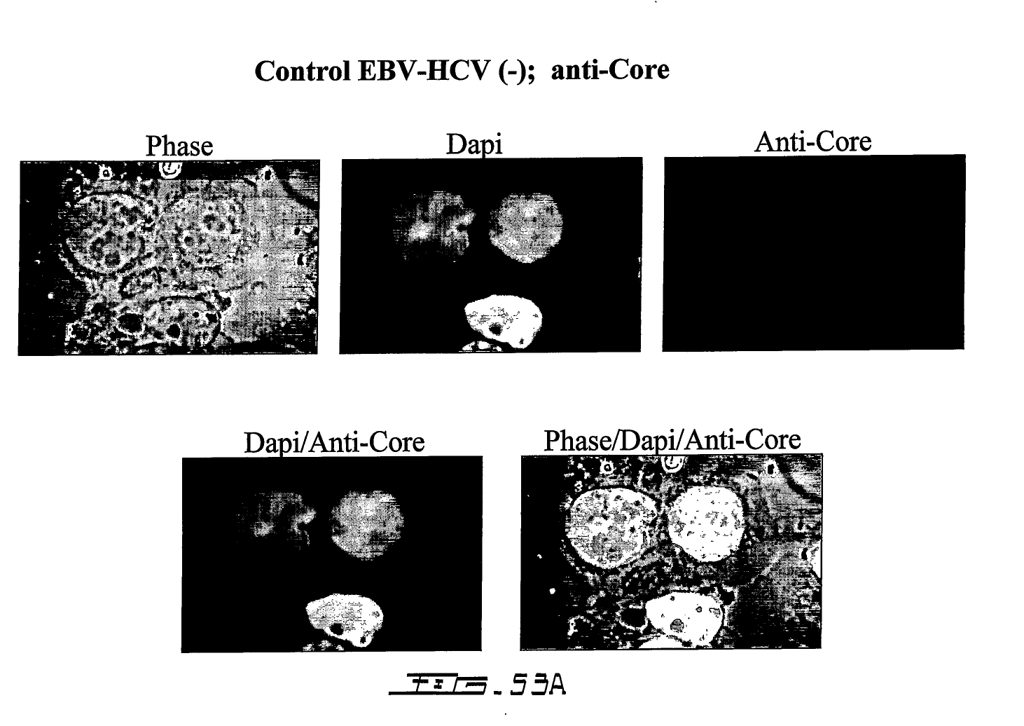

Peripheral blood lymphocytes (PBLS) obtained from an HCV negative donor can be infected by co-culturing with stimulated EBV-transformed B-cells from an HCV positive donor (FIG. 43). This implies: a) PBLs are infectable, thus HCV has tropism for these cells, b) HCV produced by the EBV-transformed B-cells from an HCV positive donor is infectious.

Non-limiting advantages of this system include:
a) EBV-transformed B-cells grow in culture. Therefore, a cell based replication system for HCV has been developed.
b) EBV-transformed B-cells proliferate under normal culture conditions (RPMI 1640, Antibiotics and 10% serum), but produce the virus only when stimulated.
c) the released virus is infectious. Therefore, this system can be used for HCV receptor identification.
d) This system should prove useful in the discovery and validation of new anti-HCV agents at all levels of the virus life cycle (entry, protein synthesis, RNA replication, assembly and release).
   XXII. Established EBV-Transformed Single Cell Clones Enabling Robust Hepatitis C Virus Replication and Characterization of the HCV Harbored Within

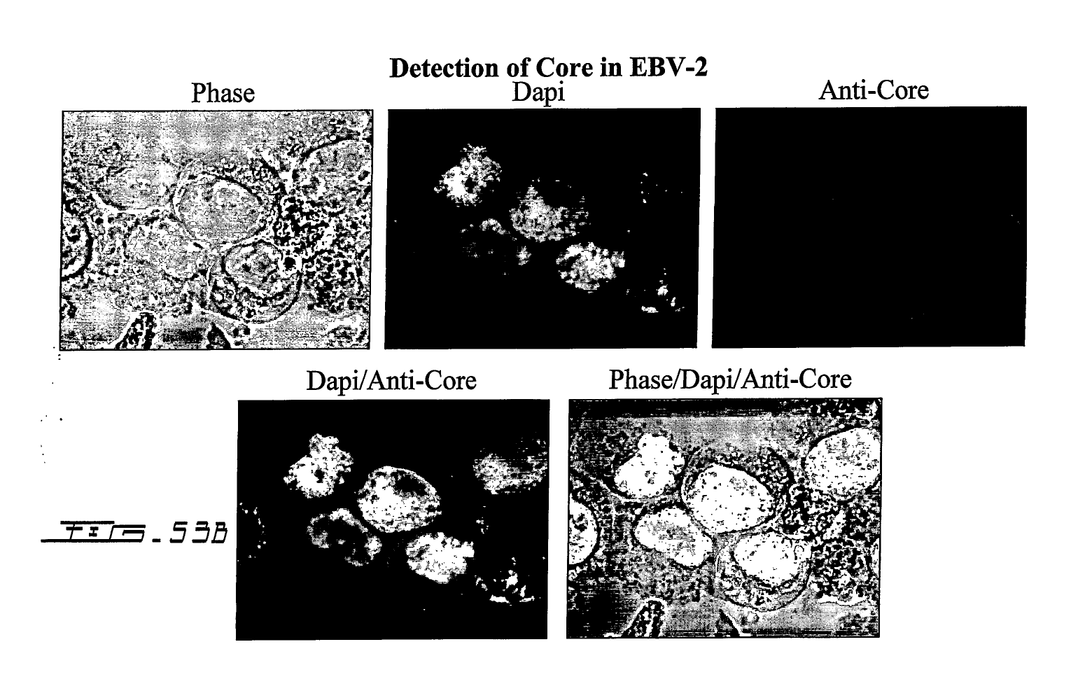

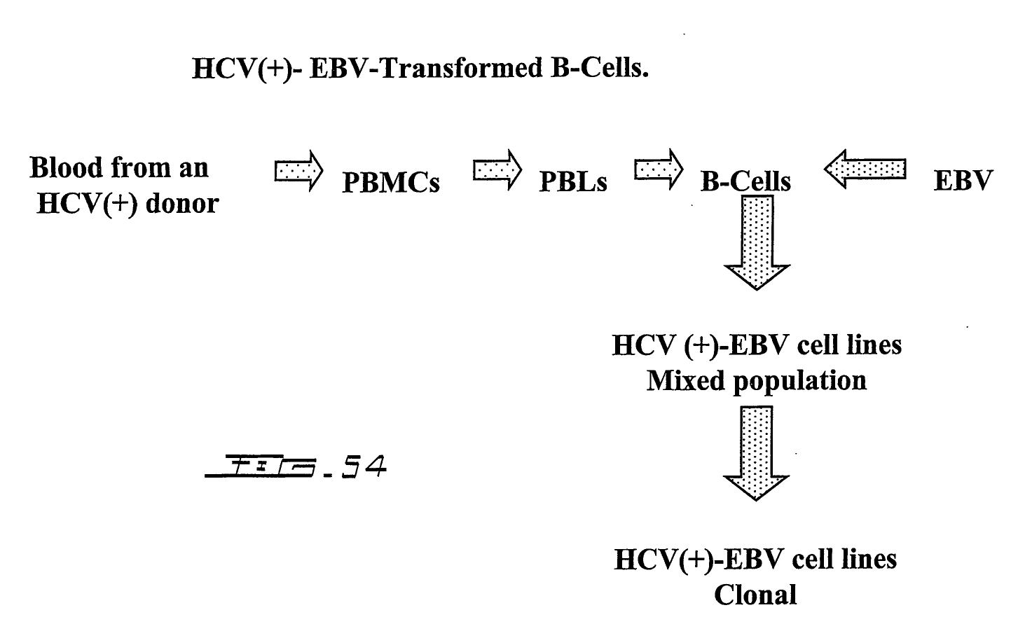

Thus, it has been demonstrated that HCV can be grown using a co-culture system assay. It has also been shown that HCV can be actively and fully expressed in immortalized cell lines. Furthermore methods of actively producing HCV in vitro have been taught (see FIG. 50, for an overview).

Using established B-cell lines of the present invention, further selection of single cell clones and the characterization of the virus harbored within were carried out. Such a characterization demonstrates the power and versatility of the present invention. It also demonstrates how the structure-function relationship of HCV can be scrutinized.

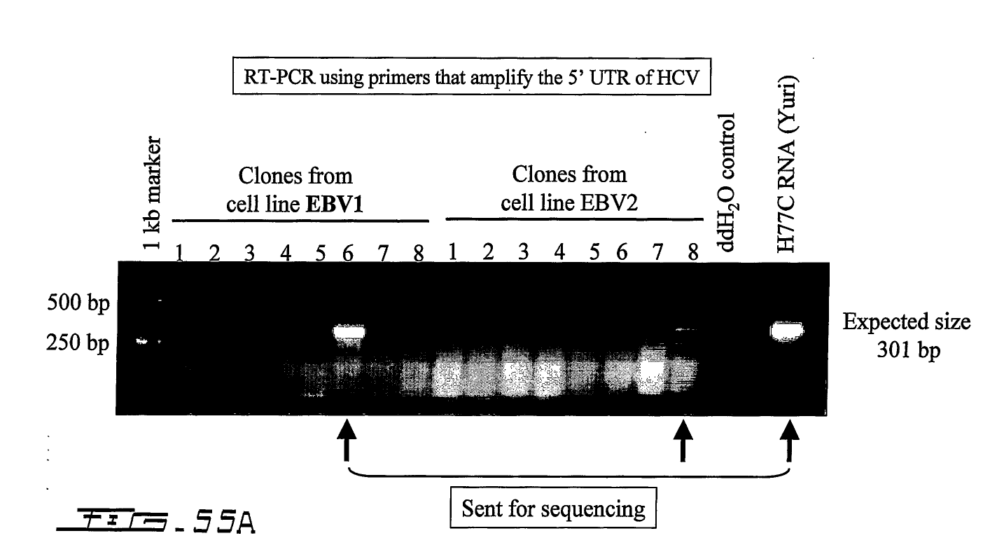

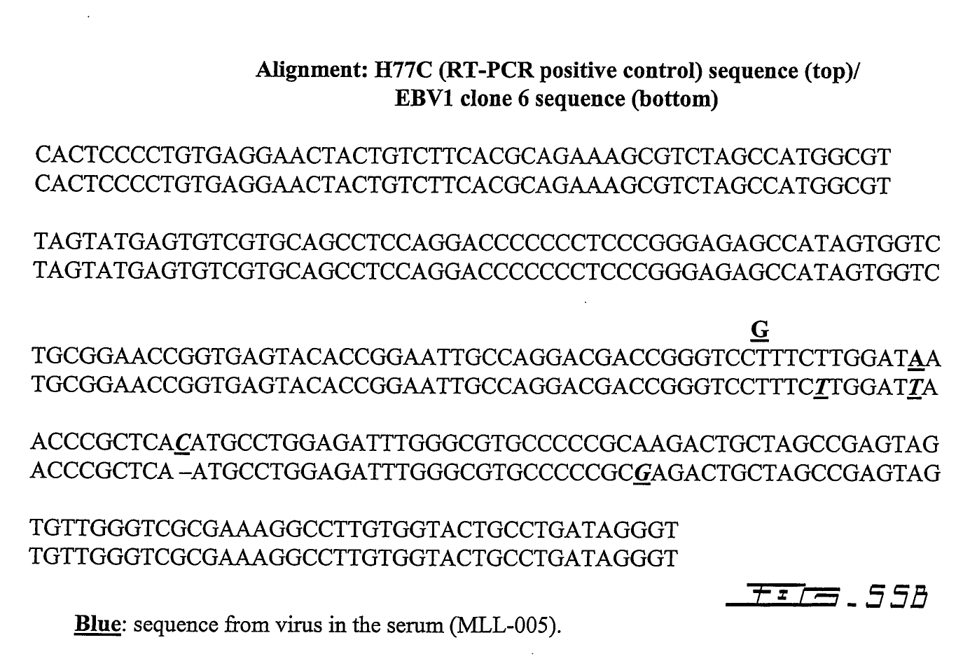

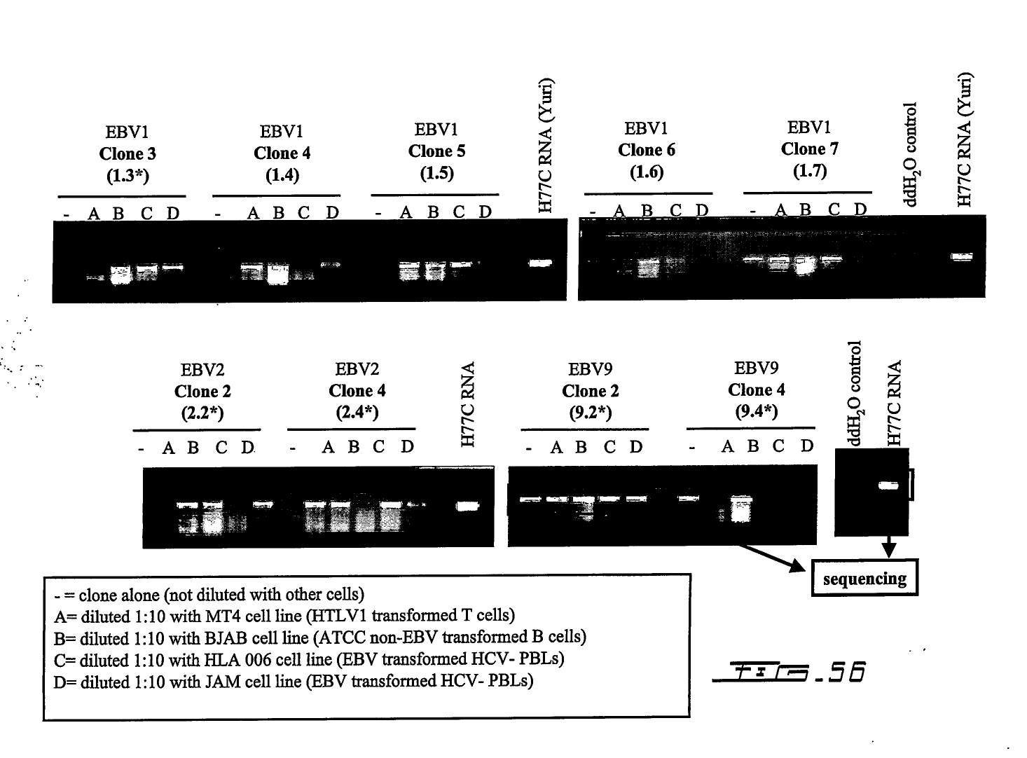

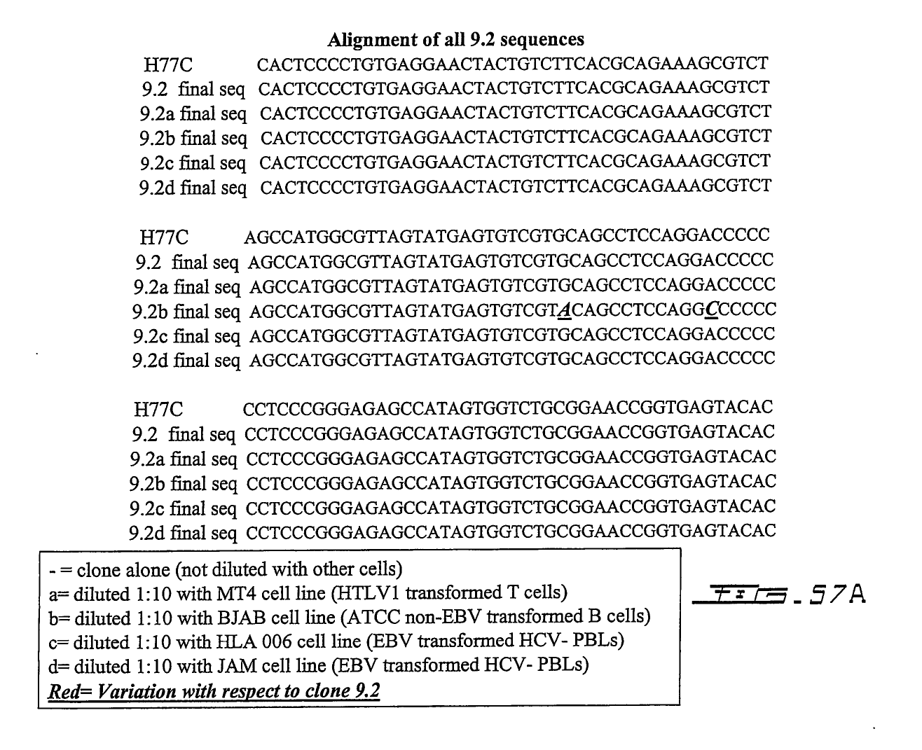

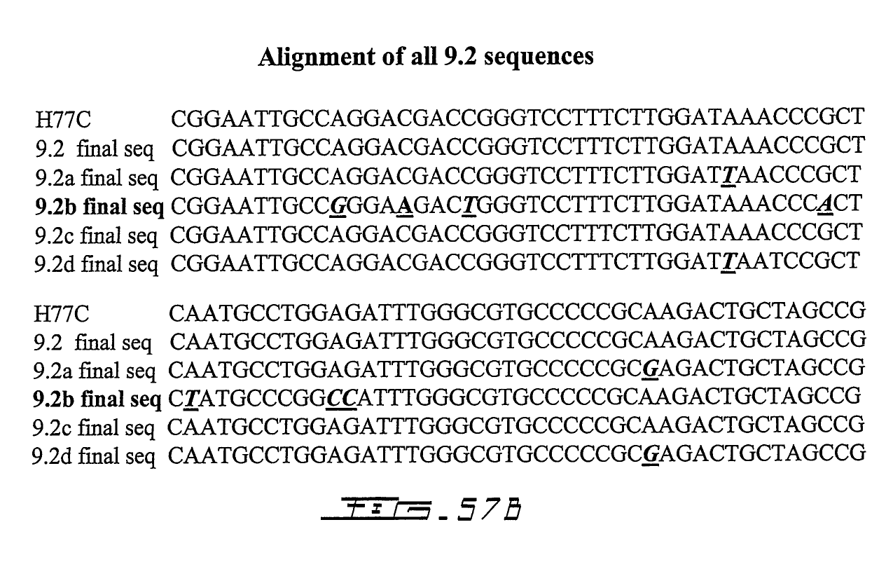

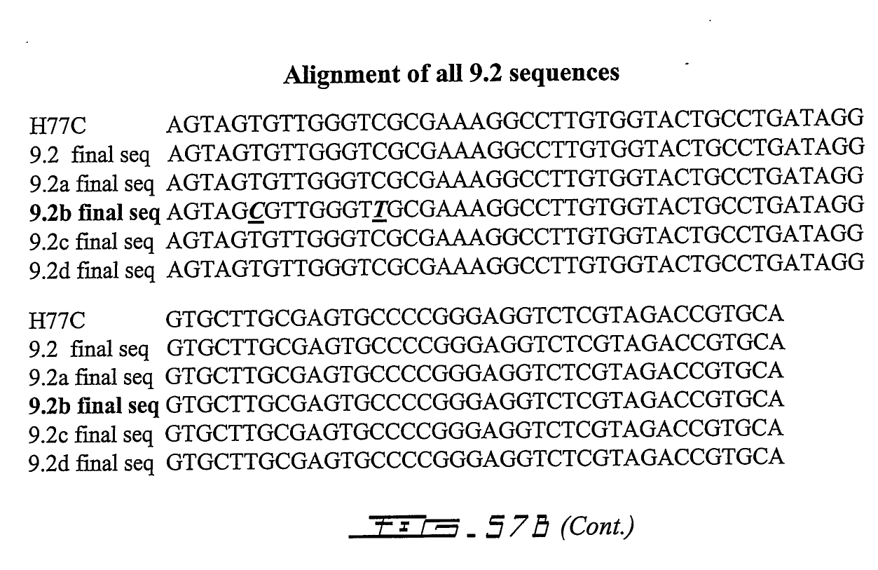

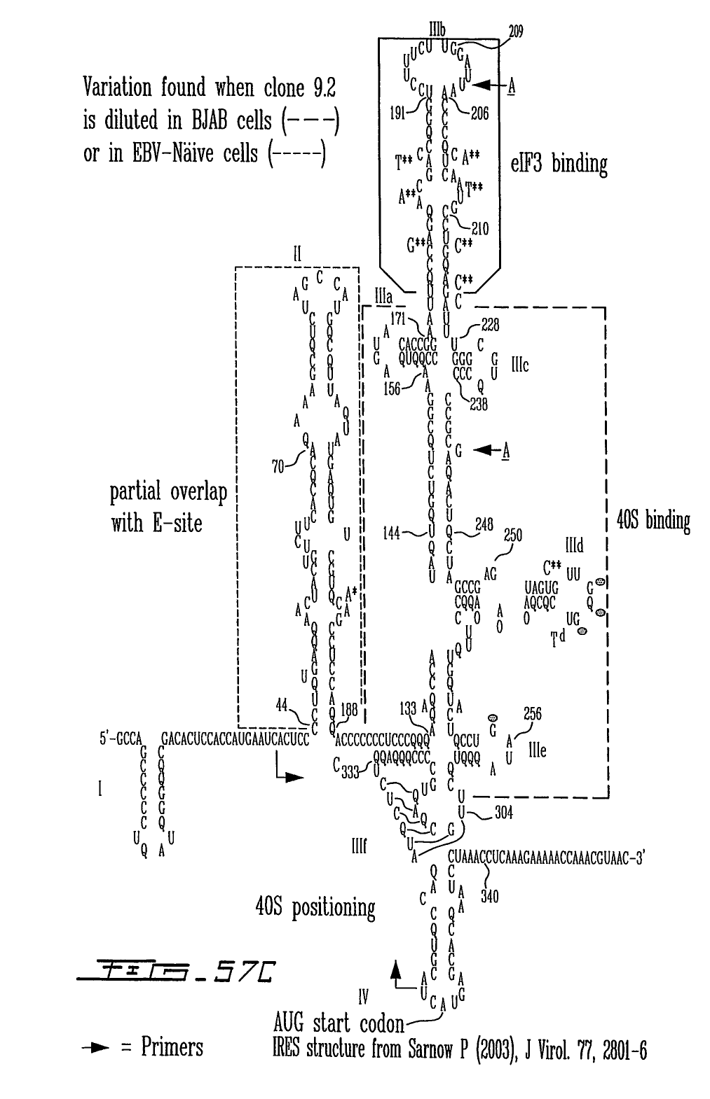

XXIII. Methods (With Reference to FIGS. 50-57).

Real Time RT-PCR

Reverse transcription was carried out at 50° C. for 20 minutes in a one-tube two-step RT-PCR reaction with Thermoscript reverse transcriptase (Invitrogen), 10 μM of HCV-tagged strand-specific RT primer and 100 μM of anti-sense GAPDH primer. The reverse transcriptase was inactivated by heating for 5 minutes at 95° C. and PCR (22 cycles) with Platinum™ Taq DNA polymerase was performed in a Trio-thermocycler™ (Biometra): at 94° C. for 45 s, 60° C. for 60 s, 68° C. for 2 min. The first round PCR products were then amplified for 40 cycles in the Roche LightCycler™ instrument: denaturation at 950C for 60 s, and amplification and quantification at 95° C. for 15 s, 60° C. for 10 s with a single fluorescence measurement, 72° C. for 15 s. Real-time quantification of RNA copy numbers for HCV and the human GAPDH gene was based on a set of eight log₁₀ external standards covering 10⁸ to 10¹ plasmid copies of a pCRII vector containing the 5′ HCV leader (genotype 1a) and the GAPDH normalization PCR amplicons which were run in parallel with the test samples. RNA extracted from PBLs of a HCV negative donor was used as control. As a reaction control for the strand-specific signal, the RT step of the RT-PCR was carried out without a HCV-tagged primer.

Immunofluorescence

Immunofluorescence was performed on 5×10⁴ cells. Following cytospin™ for 7 min at 1100 rpm in a Cytosin™ 2 (Shandon), cells were dried for 30 min at room temperature and fixed for 30 min at −20° C. in a mixture of acetone and methanol (1:1 vol/vol). Cells were blocked for 30 min at room temperature in 10 mM Tris-HCl pH 8.0 containing 1% BSA. Slides were washed 3 times with PBS and incubated at room temperature for 2 h with the polyclonal anti-core RR8 antibody (1/50) in a humidified box. Slides were washed 3 times with PBS. For Core detection, slides were incubated 1 h at room temperature with an Alexa-594 conjugated antibody (dilution 1/250). DAPI staining was performed for 7 min at room temperature (1 μg/ml final concentration). Mounted slides (Permount™ mounting medium, Fisher Scientific) were stored overnight at 4° C. prior to analysis. Conventional epifluorescence micrographs were obtained on a Zeiss Cell Observer™ system equipped with an Axiovert 200 M™ microscope using the 100× oil lens. Images were digitally deconvoluted with the AxioVision 3.1™ software using the Nearest Neighbor™ deconvolution method that uses the Agard's formula.

PBLs Stimulation

Mitogens were added to the media (RPMI 1640, 10% FBS, 50 IU/ml penicillin, 50 mg/ml streptomycin) upon starting the culture and maintained throughout the experiment. The protocols used for PBLs stimulation were as follows:

Method A, PBLs were grown in the presence of irradiated L4.5 cells (murine fibroblasts expressing the CD40 ligand, CD154) as described [Loembe, 2001 #1962].

Method B, 1 μg/ml of anti-CD3 and 200 U/ml of IL-2 (Sigma-Aldrich CO).

Method P, 3 μg/ml phytohemagglutinin (PHA, Sigma-Aldrich CO), and 200 U/ml IL-2. Method PS, 1:10⁴ vol/vol of Staphylococcus aureus Cowan fixed cells (SAC, Calbiochem) in combination with phytohemagglutinin and 200 U/ml IL-2.

Method S, 1:104 vol/vol of SAC and 200 U/ml of IL-4 (Sigma-Aldrich CO). Cell activation was verified by flow cytometry. Cells were rinsed twice with 1 ml cold phosphate buffered saline (PBS: 137 mM NaCl, 2.7 mM KCl, 4.3 mM Na₂HPO₄, 1.4 mM KH₂PO₄, pH 7.4) and fixed in 80% ethanol/PBS for 30 min at 4 ° C. PBS (2 volumes) was added and cells were pelleted by centrifugation. Cells were rinsed twice with 2 ml PBS and then resuspended in 0.5 mL PBS containing 0.2 μg/ml RNase A and incubated for 40 min at 37° C. Propidium iodide was added to a final concentration of 1.2 μg/ml and samples were analyzed by flow cytometry using a single laser FACS instrument (Becton-Dickinson) combined with the CellQuest™ software.

Epstein Barr virus (EBV) transformed B cell lines

B cells from HCV positive donors were transformed by infection with EBV from B95-8 marmoset cell line supernatant [Miller, 1973 #1983]. Briefly, 5×10⁶ PBLs were infected by incubation in 1 ml of B95-8 supernatant for 2 hr at 37° C. in a 5% CO₂ atmosphere. PBLs in 10 ml of media (RPMI 1640, 20% FCS, 2mM L-glutamine, 50 IU/ml penicillin, 50 mg/ml streptomycin and 50 μM 2-mercaptoethanol (Sigma), 1 μg/ml of cyclosporin A (Sandimmun, Novartis Pharmaceuticals Canada Inc., Dorval QC, Canada), transferred to 25 cm² flasks and incubated for 2 to 3 weeks before expansion. Established EBV-transformed cell lines were confirmed to be B cells (CD19 (+), CD3 (−), CD16 (−), CD56 (−)). EBV transformed B cell lines were typed for major histocompatibility complex (MHC) class I antigen expression by the amplification refractory mutation system—polymerase chain reaction (ARMS-PCR) using 95 primer sets amplifying defined MHC class I alleles (ABC SSP Unitray, Pel-Freez Clinical Systems, Brown Deer, Wis.) [Bunce, 1995 #1984]. Genomic DNA for molecular HLA-typing was prepared from EBV transformed B cell lines using the QIAamp™ DNA blood kit (Qiagen Inc., Mississauga, ON).

RNA Purification

Total RNA was extracted from cells using Trizol™ (Invitrogen) according to the manufacturer's protocol. Yeast tRNA (1 mg/ml) was added as a carrier. RNA was resuspended in nuclease-free water (Sigma-Aldrich CO). Total RNA was quantified by Phosphoimager™ (STORM system, Molecular Dynamics) using the RiboGreen™ RNA Quantification Kit (Molecular Probes, Inc). XXIV. Use of the cell lines of the invention to identify, validate or improve the antiviral activity of compounds

Non-limiting examples of candidate anti-HCV compounds (pool thereof, librairies of compounds, pool thereof . . . ) to be used in screening using the assays and cells of the present invention are presented herein. In addition, the present invention provides the means to assess the resistance/phenotype profile of patients' strains of HCV toward a particular anti-HCV compound or candidate or pool thereof.

Non-limiting examples of compounds that could be used in such phenotype determination are listed in Tables 1 and 2.

TABLE 1
Selected IFN-based therapies for the treatment of HCV infection

| | | | |
|---|---|---|---|
| Drug name | Company | Web site | Clinical phase |

Monotherapy

| | | | |
|---|---|---|---|
| Intron A (IFN-α2b, recombinant) | Schering-Plough | http://www.sch-plough.com | FDA approval, 1995 |
| PEG-INTRON (PEGylated IFN-α2b) | Schering-Plough | http://www.sch-plough.com | FDA approval, 2001 |
| Roferon A (IFN-α2b, recombinant) | Roche | http://www.roche.com | FDA approval, 1996 |
| Pegasys (PEGylated IFN-α2b) | Roche | http://www.roche.com | FDA approval, 2001 |
| Infergen A (IFN alfacon-1) | InterMune Pharmaceuticals | http://www.intermune.com | FDA approval, 1997 |
| Wellferon (lymphoblastoid IFN-αn1) | GlaxoSmithKline | http://www.corp.gsk.com | FDA approval, 1999 |
| Omniferon (natural IFN-α) | Viragen (Scotland) | http://www.viragen.com | Phase II |
| Omega IFN(IFN-ω) | BioMedicines | http://www.biomedicinesinc.com | Phase II |
| Albuferon-α (albumin-IFN-α2b) | Human Genome Sciences | http://www.hgsi.com | Phase I |
| Rebif (IFN-β1a) | Serono | http://www.serono.com | Preclinical* |

Combination Therapies

| | | | |
|---|---|---|---|
| Rebetron (Intron A and ribavirin) | Schering-Plough | http://www.sch-plough.com | FDA approval, 1998 |
| PEG-INTRON and ribavirin | Schering-Plough | http://www.sch-plough.com | FDA approval, 2001 |
| Pegasys and ribavirin | Roche | http://www.roche.com | FDA application |
| | | | submitted |
| Intron A and Zadaxin (α1-thymosin) | RegeneRx Biopharmaceuticals/ | http://www.regenerx.com | Phase III |
| | SciClone Pharmaceuticals | http://www.sciclone.com | |
| Pegasys and Ceplene | Maxim Pharmaceuticals | http://www.maxim.com | Phase III |
| IFN-β and EMZ701 | Transition Therapeutics | http://www.transitiontherapeutics.com | Preclinical |

*FDA approval for the treatment of relapsing forms of multiple sclerosis. HCV, hepatitis C virus; IFN, interferon; PEG, polyethylene glycol

TABLE 2
A sample of the drug pipeline for hepatitis C and related treatments

| Target/ | | Mechanism/ | | Clinical |
|---|---|---|---|---|
| indication | Drug name | drug category | Company | Phase |
| IRES | ISIS 14803 | Antisense | ISIS Pharmaceuticals/ | Phase II |
| | | | Elan Corporation | |
| | Heptazyme | Ribozyme | Ribozyme Pharmaceuticals | Phase II* |
| NS3 | BILN-2061 | Serine-protease inhibitor | Boehringer Ingelheim | Phase II |
| | VX-950/LY-570310 | Serine-protease inhibitor | Vertex Pharmaceuticals/Lilly | Preclinical |
| NS5B | JTK-003 | RdRp inhibitor | Japan Tobacco | Phase I/II |
| E1 | Not known; a recombinant E1 | Therapeutic vaccine | Innogenetics | Phase IIa |
| E2 | XTL-002 | Monoclonal antibody | XTL Biopharmaceuticals | Phase Ib |
| IMPDH | VX-497 | IMPDH inhibitor | Vertex Pharmaceuticals | Phase II |
| | Levovirin | IMPDH inhibitor | Ribapharm | Phase I |
| | Viramidine | IMPDH inhibitor | Ribapharm | Phase I |
| Liver fibrosis | Actimmune (IFN-γ) | Antifibrotic | InterMune Pharmaceuticals | Phase II |
| | IP-501 | Antifibrotic | Interneuron Pharmaceuticals | Phase III |
| Liver apoptosis | IDN-6556 | Caspase inhibitor | Idun Pharmaceuticals | Phase II |
| HCC | T67 | β-tubulin inhibitor | Tularik | Phase III |
| HCV re-infection | Civacir | HCV IgG | Nabi Pharmaceuticals | Phase I/II |
| | CellCept (Mycophenolate mofetil) | Immunosuppressant | Roche Holdings | Preclinical |
| Target unknown | Ceplene (histamine dihydrochloride) | Immune modulator | Maxim Pharmaceuticals | Phase II |
| | Zadazin (thymosin α-1) | Immune modulator | SciClone Pharmaceuticals | Phase III |
| | Symmetrel (amantadine hydrochloride) | Broad antiviral agent | Endo Laboratories | Phase IV |

*Suspended pending toxicology investigation. HCC, hepatocellular carcinoma; HCV, hepatitis C virus; IFN, interferon; IgG, immunoglobulin G; IMPDH, inosine monophosphate dehydrogenase; IRES, internal ribosome-entry site; NS, non-structural protein; RdRp, RNA-dependent RNA polymerase.

### CONCLUSIONS

The present invention relates among other things to the fact that:
(1) HCV has PBMC tropism;
(2) HCV can naturally infect blood cells;
(3) HCV can replicate in PBMCs and PBMLs;
(4) HCV replicating in naturally infected PBMCs is infectious;
(5) HCV can replicate in extrahepatic tissue; and
(6) HCV has a latent phase during PBMC infection, which can be ended by activation.

It is interesting to note that HCV replication is activated upon immune response. Thus, a person of ordinary skill in the art will be able to provide other methods of activation than those disclosed herein (or complementary thereto) to activate HCV replication in PBMCs or PBLCs, without undue experimentation.

The present invention provides the tools to study hepatitis C virus replication in a simple cell culture based system. This simple culturing tool is suitable for the search and validation of novel HCV antiviral drugs and therapies (vaccine). The assays and methods of the present invention enable the performance of screening assays to identify antiviral agents. Of course, the assays can be highthroughput. Compound libraries can now be used to identify candidate anti-HCV agents. These assays can thus be used to generate lead compounds for pharmaceutical anti-HCV formulations.

The novel replication system of the present invention, in one embodiment, based on PBMCs (or PBMLs) is simple, does not require facilities other than those normally used for HIV research, and allows experiments with the complete HCV. Thus, novel drugs and therapies can be screened to target all the different stages of virus replication such as virus entry, cytoplasmic replication (viral (−) and (+) strand synthesis), viral protein synthesis, virus assembly, virus trafficking, and virus release.

Although the present invention has been described hereinabove by way of preferred embodiments thereof, it can be modified without departing from the spirit and nature of the subject invention as defined in the appended claims.

### REFERENCES

1. Journal Of Viral Hepatitis 6, 35-47. (1999).
2. J. H. Hoofnagle, Hepatology26, 15S-20S (1997).
3. J. G. McHutchison, M. W. Fried, Clin Liver Dis 7,149-61. (2003).
4. S. L. Tan et al., Nat Rev Drug Discov 1, 867-81. (2002).
5. R. Bartenschlager, V. Lohmann, J Gen Virol81, 1631-48. (2000).
6. K. E. Reed, et al., Curr. Top. Microbiol. Immunol. 242, 55-84. (2000).
7. Z. Xu et al., EMBO J 20, 3840-8. (2001).
8. J. Gomez, Et al., Journal Of Viral Hepatitis 6, 3-16. (1999).
9. A. L. Zignego, et al., Journal Of Hepatology 31, 369-76. (1999).
10. J. A. Hellings, et al., J Virol Methods 10, 321-6. (1985).
11. J. P. Bronowicki et al., Hepatology 28, 211-8. (1998).
12. J. Boisvert et al., Journal Of Infectious Diseases 184, 827-35. (2001).
13. A. L. Zignego et al., Journal Of Hepatology 15, 382-6. (1992).
14. J. T. Wang, et al., Journal Of Hepatology 16, 380-3 (1992).
15. H. M. Muller et a., J Gen Virol 74, 669-76 (1993).
16. J. Bartolome, et al., J Exp Med 178, 17-25 (1993).
17. Y. K. Shimizu et al, J Virol 71, 5769-73 (1997).
18. Y. K. Shimizu et al., Infection 26, 151-4 (1998).
19. E. G. Westaway, et al., Virology 258, 108-17. (1999).
20. K. Yasui et al., J Virol 72, 6048-55. (1998).
21. T. Pietschmann et al., J Virol 76, 4008-4021 (2002).
22. H. J. Ezelle, et al., J Virol 76, 12325-34. (2002).
23. W. Pumeechockchai et al, Journal Of Medical Virology 68, 335-42. (2002).
24. T. F. Baumert, et al., J Virol 72, 3827-36. (1998).
25. D. Bradley et al., Journal Of Medical Virology 34, 206-8. (1991).
26. M. Kaito et al., J Gen Virol 75, 1755-60. (1994).
27. M. Hijikata et al., J Virol67, 1953-8 (1993).
28. R. J. Carrick, et al., J Virol Methods 39, 279-89. (1992).
29. P. Maillard et al., J Virol 75, 8240-50. (2001).
30. Y. K. Shimizu, et al., Proc. Natl Acad. Sc. 89, 5477-81 (1992).
31. T. Mizutani, et al., Biochem.Biophys.Res. Comm. 227, 822-6 (1996).
32. T. Mizutani et al., J Virol 70, 7219-23 (1996).
33. M. Ikeda et al., Journal Of Hepatology 27, 445-54 (1997).
34. M. P. Nair et al, Clin Diagn Lab Immunol 4, 127-32. (1997).
35. T. Pellegrino, et al., J Neuroimmunol 83,139-47. (1998).
36. H. Friedman, et al., Clin Microbiol Rev 16, 209-19. (2003).
37. T. Laskus, et al., Am J Gastroenterol 93, 2162-6 (1998).
38. T. Laskus et al., Journal Of Infectious Diseases 181, 442-8 (2000).
39. M. Radkowski, et al., Hepatology 28, 1110-6 (1998).
40. B. Rouveix, Therapie 47, 503-12. (1992).
41. G. C. Baldwin, et al., J Neuroimmunol 83, 133-8. (1998).
42. M. Resti et al., Clin Infect Dis 35, 236-9. (2002).
43. F. Schwarzmann, et al., Int J Mol Med 1, 137-42. (1998).
44. F. Schwarzmann, et al., Leuk Lymphoma 30,123-9. (1998).
45. H. Yoshiyama, et al., EMBO J 14, 3706-11. (1995).
46. H. Kanegane et al., Leuk Lymphoma 34, 603-7. (1999).
47. V. Lohmann et al., Science 285,110-3. (1999).
48. D. Moradpour et al., J Biol Chem 277, 593-601. (2002).
49. M. M. Loembe, et al., Br J Haematol 113, 699-705. (2001).
50. G. Haukenes, et al., Biotechniques 22, 308-12. (1997).

1. A method for generating an established cell line which enables the replication of complete and infectious hepatitis C virus (HCV) comprising transforming peripheral blood mononuclear cells (PBMCs) which produce HCB with Epstein Barr virus (EBV).

2. The method of claim 1, wherein said cells producing HCV, are in a peripheral blood lymphocyte (PBL) fraction of said PBMCs.

3. The method of claim 2, wherein said calls producing HCV, are B-cells.

4. (canceled)

5. A method for producing HCV in vitro comprising:
a) generating an established cell line which enables the replication of complete and infectious hepatitis C virus (HCV) comprising transforming a B-cell which produces HCV, with Epstein Barr virus (EVB);
b) growing the EBV-immortalized B-cell obtained in a) in culture under conditions enabling complete and infectious HCV production.

6. The method of claim 5, wherein conditions in b) comprise a stimulation of said EBV-immortalized B-cell to produce HCV.

7. The method of calim 5, wherein said B-cell is present in a PBMC or PBL.

8. The method of claim 7, wherein said PBMC or PBL fraction is obtained from a HCV positive patient which is immunosuppressed.

9. The method of claim 8, wherein said inmunosuppression is due to injection drug use of said patient.

10. The method of claim 7, wherein said PBMC or PBL fraction is obtained from a HCV positive patient which has not been treated with interleukin.

11. The method of claim 5, further comprising a co-cultivation of said EBV-immortalized B-cell, which is stimulated to produce HCV in the presence of monocyte-derived dendritic cells (DCs).

12. (canceled)

13. An EBV-established B-cell line capable of replicating complete and infectious HCV obtained by the method of claim 1.

14. A cell-based in vitro replication system for HCV comprising an EBV-transformed B-cell capable of replicating complete and infectious HCV, and a second cell population having HCV tropism and in which robust HCV replication occurs, so that under appropriate culture conditions said second cell population can become infected by said infectious HCV produced by said EBV-transformed B-cell, and wherein said second cell population is a naïve cell population.

15. The cell-based replication system of claim 14, further comprising appropriate culture media reagents conditions enabling infection of said second call population.

16. The cell-based replication system of claim 14, wherein said second cell population is peripheral blood lymphocytes (PBLs).

17. The cell-based replication system of claim 14, wherein said second cell population is a cell line.

18. An assay for screening a test agent and selecting an agent which possesses anti-HCV activity, comprising:
using said cell-based in vitro replication system of claim 14 so as produce HCV from said second cell population; and
assaying a biological function of said HCV produced from said cell line or said cell population.

19. The assay of claim 18, wherein said biological function is selected from the group consisting of binding to a cellular receptor of HCV, replication, translation, assembly, and infectivity.

20. (canceled)

21. (canceled)
